(19) United States
(12) Patent Application Publication
(10) Pub. No.: US 2022/0307052 A1
(43) Pub. Date: Sep. 29, 2022

(54) RECOMBINANT HERPESVIRALES VECTOR

(71) Applicant: Solid Biosciences Inc., Cambridge, MA (US)

(72) Inventors: Peter Pechan, Newton, MA (US); Alexey Vladimirovich Seregin, Westford, MA (US)

(21) Appl. No.: 17/614,110

(22) PCT Filed: Jun. 1, 2020
(86) PCT No.: PCT/US2020/035558

§ 371 Date: Nov. 24, 2021

Related U.S. Application Data
(60) Provisional application No. 62/873,094, filed on Jul. 11, 2019.
(60) Provisional application No. 62/854,637, filed on May 30, 2019.

(51) Int. Cl. C12N 15/86; C12N 7/00; C12N 5/071; C12N 15/90

(52) U.S. Cl. CPC C12N 15/86; C12N 7/00; C12N 5/0686; C12N 15/90; C12N 2710/16622; C12N 2710/16644; C12N 2710/16662; C12N 2750/14122; C12N 2750/14142; C12N 2750/14152; A61K 48/00

(57) ABSTRACT

The invention described herein provides a recombinant replication-defective vims derived from Herpesvirales order, wherein the virus is characterized by a complete deletion of a gene encoding ICP27, or a functional equivalent gene thereof. The invention also provides production cell lines for such recombinant replication-defective vims, wherein the cell lines have a coding sequence for ICP27 or a functional equivalent thereof, and wherein the coding sequence has no or minimal sequence overlap with the virus characterized by the complete deletion of the gene encoding ICP27. Method of using such recombinant replication-defective vims and production cell lines are also provided.

### REFERENCE TO RELATED APPLICATIONS

This application claims the benefit of the filing date of U.S. Provisional Patent Application Nos. 62/854,637, filed on May 30, 2019, and 62/873,094, filed on Jul. 11, 2019, the entire contents of each of the above applications are incorporated herein by reference.

### BACKGROUND OF THE INVENTION

Recombinant adeno-associated viral (rAAV) vectors production using co-infection with recombinant herpes simplex virus type 1 (rHSV) vectors is a very efficient method of generating a large amount of rAAV particles (Conway et al., Gene Therapy 6:986-993, 1999; Booth et al., Gene Therapy 11:829-837, 2004; Adamson-Small et al., Mol. Ther. Med. Clin. Dev. 3:16031, 2016). The production method relies on the role played by the HSV in AAV life cycle as a helper virus for replication in permissive cells. Therefore, the rHSV virus can serve both as a helper and as a shuttle to deliver the necessary AAV functions that support AAV genome replication and packaging to the producing cells.

Some rHSV vectors for this system are engineered from a replication-deficient variant of HSV type 1 virus d27-1 with a 1.6-kb deletion in a gene encoding the viral Infected Cell Protein 27, or ICP27 (Rice and Knipe, J. Virol. 64:1704-1715, 1990). ICP27 is a 512-amino acid protein, also known by names based on its molecular weight, as Immediate Early 63 (IE63) or Viral Molecular Weight 63 (Vmw63) protein, or by its location on HSV-1 genome as Unique Long 54 (UL54) gene. ICP27 is one of the first proteins to be expressed in cells infected with HSV-1 and is absolutely essential for viral replication in cell culture. In the absence of ICP27, the rHSV genome cannot replicate, unless ICP27 is provided in trans, for example, by V27 cells, a Vero cell derivative stably transformed with 2.4-kb BamHI-HpaI fragment containing UL54 and part of UL55 genes to express ICP27 (Rice and Knipe, J. Virol. 64:1704-1715, 1990). Other reported ICP27-complementing cell lines were Vero-derived 2-2 cells and BHK21-derived B130 cells, both stably transformed with 2.4-kb BamHI-SstI fragment also containing UL54 and part of UL55 genes (Smith et al. Virology 186: 74-86, 1992; Howard et al. Gene Therapy 5:1137-1147, 1998).

V27 is the only cell line currently used for large-scale manufacturing of rHSV stocks used for rAAV production (Penaud-Budloo et al., Mol. Ther.: Med. & Clin. Dev. 8:166-180, 2018). rHSV stocks are prepared by infecting monolayers of V27 cells in flasks, or alternatively, in suspension using micro-carriers. The resulting rHSV stocks are harvested after 3-4 days, and concentrated (Knop and Harell, Biotechnol. Prog. 23:715-721, 2007; Adamson-Small et al., Mol. Ther. Med. Clin. Dev. 3:16031, 2016).

However, whenever a replication-defective virus vector is propagated in a helper cell line, there is a high probability that the final stock will contain a sub-population of virus that has obtained replication competency through the process of homologous recombination (HR) between the viral genome and the integrated viral gene(s) present in the cellular genome observed also during production of adenovirus (Ad) vectors, where homologous recombination between E1 gene-deleted Ad vectors genomes and the integrated E1 sequences in 293 cells frequently resulted in replication-competent Ads (RCA) that contaminated stocks of E1-vectors (Hehir et al., Journal of Virology. 70:8459-8467, 1996). It is no exception that the manufacture process using the current d27-1 recombinant HSV (rHSV) vector in V27 cells can also lead to the generation of a “wild-type like” replication-competent HSV (rcHSV) contamination of the rHSV lots.

Specifically, replication-competent HSV (rcHSV), or ICP27 revertants, may arise during amplification of rHSV in V27 by homologous recombination (Ye et al., Hum. Gene Ther. Clin. Dev. 25:212-217, 2014). Low levels of rcHSV have been shown in rHSV stocks, reported less than 1 PFU per 3×10⁸ rHSV PFUs (Penaud-Budloo et al., Mol. Ther.: Med. & Clin. Dev. 8:166-180, 2018).

However, any rcHSV virus, which would behave phenotypically as wild-type virus, would pose a serious problem to the therapeutic use of replication-defective stocks, because HSV-1 has the potential for uncontrolled viral replication and has a potential to cause a crippling form of encephalitis as a result of viral spread in the brain (Asenbauer et al., Neuropediatrics 29:120-123, 1998; Gurses et al., Annals of Tropical Paediatrics. 16:173-175, 1996; Yamada et al. Journal of Neurology, Neurosurgery & Psychiatry 74:262-264, 2003).

Thus, there is a need to improve the existing rHSV production process that utilizes the d27-1 rHSV vector.

### SUMMARY OF THE INVENTION

The vectors described herein provides new rHSV vectors with a larger, relatively complete ICP27 gene deletion in its backbone, compared to the existing d27-1 ICP27 deletion, and obviates contamination of rcHSV generated during the production of rHSV using the d27-1-based complementation system in a rHSV production cell line. Indeed, similar viral vectors in other viruses derived from the Herpesvirales order are also part of the invention.

The invention described herein also provides recombinant vectors that can be introduced into suitable viral vector (e.g., rHSV) production cell lines, wherein the recombinant vectors provide the requisite ICP27 coding sequence for propagating the viral (e.g., rHSV) vectors of the invention in the host cell. In certain embodiments, there is little or no overlap in the ICP27 coding sequence in the recombinant vectors (which may be integrated into the genome of the viral production cell line), and the subject viral (e.g., rHSV) vectors having the larger, relatively complete ICP27 deletion.

The invention further provides host cells comprising such recombinant vectors.

The invention further provides methods of propagating/amplifying/producing the subject viral vectors (e.g., rHSV vectors).

The invention further provides methods of producing recombinant Adeno-Associated Virus (rAAV) using the subject viral vectors (such as rHSV).

Thus one aspect of the invention provide a recombinant replication-defective virus derived from the Herpesvirales order, wherein the virus is characterized by a deletion in a gene encoding ICP27, or a functional equivalent gene thereof, wherein the deletion is at least 1,200 bps in length and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp of the most 3′-end of the gene encoding ICP27 (e.g., SEQ ID NO: 11), or the functional equivalent gene thereof.

In certain embodiments, the recombinant replication-defective virus is derived from a non-clinical or laboratory virus from the Herpesvirales order.

In certain embodiments, the deletion comprises, consisting essential of, or consisting of the entire coding sequence (or ORF) of the gene encoding ICP27 or the functional equivalent gene thereof.

In certain embodiments, the deletion further comprises the entire promoter region of the gene encoding ICP27 or the functional equivalent gene thereof, or a portion (e.g., the most 3′ about 400 nucleotides) of the promoter region.

In certain embodiments, the gene encoding ICP27 has the polynucleotide sequence of SEQ ID NO: 11.

In certain embodiments, the virus is derived from the Alloherpesviridae family or the Malacoherpesviridae family.

In certain embodiments, the virus is derived from the Herpesviridae family.

In certain embodiments, the virus is derived from the Alphaherpesvirinae subfamily, the Betaherpesvirinae subfamily, or the Gammaherpesvirinae subfamily.

In certain embodiments, the virus is derived from HHV-1 (Herpes Simplex Virus-1 or HSV-1), HHV-2 (Herpes Simplex Virus-2 or HSV-2), HHV-3 (Varicella Zoster Virus or VZV), HHV-4 (Epstein-Barr Virus or EBV), HHV-5 (Cytomegalovirus or CMV), HHV-6A/HHV-6B (Roseolovirus, Herpes Lymphotropic Virus), HHV-7, or HHV-8 (Kaposi's Sarcoma-Associated Herpesvirus or KSHV).

In certain embodiments, the virus is derived from Cercopithecine herpesvirus-1 (CeHV-1) or Murid Herpesvirus 68 (MHV-68 or MuHV-4).

In certain embodiments, the virus is derived from porcine Alpha-herpesviruses, including pseudorabies virus (PRV).

In certain embodiments, the virus is derived from the Simplexvirus genus, such as Ateline herpesvirus 1, spider monkey herpesvirus, Porcine herpesviruses, Bovine herpesvirus 2, Cercopithecine herpesvirus 1 (Herpes B virus), Fruit bat alphaherpesvirus 1, Leporid herpesvirus 4, Macacine herpesvirus 1, Macropodid herpesvirus 2, & Papiine herpesvirus 2.

In certain embodiments, the virus is derived from the Varicellovirus genus, such as Bovine herpesvirus 1, Bovine herpesvirus 5, Bubaline herpesvirus 1, Caprine herpesvirus 1, Canine herpesvirus 1, Cercopithecine herpesvirus 9, Cervid herpesvirus 1, Cervid herpesvirus 2, Elk herpesvirus 1, Equine herpesvirus 1, Equine herpesvirus 3, Equine herpesvirus 4, Equine herpesvirus 8, Equine herpesvirus 9, Feline herpesvirus 1, and Suid herpesvirus 1.

In certain embodiments, the virus is derived from the Mardivirus genus, such as Anatid herpesvirus 1, Columbiform herpesvirus 1, Gallid herpesvirus 2, Gallid herpesvirus 3 (GaHV-3 or MDV-2), Meleagrid herpesvirus 1 (HVT), and Peacock herpesvirus 1.

In certain embodiments, the virus is derived from the Litovirus genus, such as Gallid herpesvirus 1, and Psittacid herpesvirus 1.

In certain embodiments, the virus is derived from a reptilian Alphaherpesvirus, such as Caretta caretta herpesvirus, Chelonid herpesvirus 1, Chelonid herpesvirus 2, Chelonid herpesvirus 3, Chelonid herpesvirus 4, Chelonia mydas herpesvirus, Coober herpesvirus, Emydid herpesvirus 1, Emydid herpesvirus 2, Fibropapilloma associated herpes virus, Gerrhosaurid herpesvirus 1, Gerrhosaurid herpesvirus 2, Gerrhosaurid herpesvirus 3, Glyptemis herpesvirus 1, Glyptemys herpesvirus 2, Iguanid herpesvirus 1, Iguanid herpesvirus 2, Loggerhead orocutaneous herpesvirus, Lung-eye-trachea associated herpesvirus, Pelomedusid herpesvirus 1, Red eared slider herpes virus, Terrapene herpesvirus 1, Terrapene herpesvirus 2, Testudinid herpesvirus 1, Testudinid herpesvirus 2, Testudinid herpesvirus 3, Testudinid herpesvirus 4, and Varanid herpesvirus 1.

In certain embodiments, the virus is derived from the Rhadinovirus genus, such as Alcelaphine herpesvirus 1, Alcelaphine herpesvirus 2, Ateline herpesvirus 2, Bovine herpesvirus 4, Cercopithecine herpesvirus 17, Equine herpesvirus 2, Equine herpesvirus 5, Equine herpesvirus 7, Japanese macaque rhadinovirus, Leporid herpesvirus 1, and Murid herpesvirus 4 (Murine gammaherpesvirus-68 or MHV-68).

In certain embodiments, the virus is a laboratory strain of HSV-1, such as KOS, KOS 1.1, KOS 1.1A, KOS63, KOS79, McKrae, Stain 17, F17, McIntyre, or others.

In certain embodiments, the functional equivalent gene thereof is ORF57 of KSHV, Mta/SM/EB2 of EBV, UL69 of human CMV, or other equivalent genes in any viruses from the Herpesvirales order.

In certain embodiments, the recombinant replication-defective virus of the invention further comprises a coding sequence for AAV Rep and Cap proteins, and/or a gene of interest (GOI) flanked by AAV ITR sequences.

In certain embodiments, the coding sequence for the AAV Rep and Cap proteins, and/or the gene of interest (GOI) flanked by AAV ITR sequences is integrated into or replaces a non-essential gene of the virus (e.g., not required for viral replication and not required for viral packaging).

Another aspect of the invention provides a recombinant vector capable of expressing ICP27 or a functional equivalent thereof in a host cell, the vector comprising: (1) a coding sequence for the ICP27 or the functional equivalent thereof, operatively linked to a promoter capable of directing the transcription of the coding sequence in the host cell; (2) a polyadenylation site 3′ to the coding sequence; and, (3) optionally, one or more multi-cloning site(s); wherein the vector contains no more than 300 bp, 250 bp, 200 bp, 150 bp, 100 bp, 50 bp, 30 bp, 20 bp, 10 bp, 9 bp, 8 bp, 7 bp, or 6 bp consecutive nucleotides of any one of the virus of the invention.

In certain embodiments, the ICP27 has the amino acid sequence of SEQ ID NO: 10, or is at least 60%, 70%, 75%, 80%, 85%, 90%, 95%, 97%, 98%, 99%, 99.2%, 99.4%, 99.6%, or 99.8% identical to SEQ ID NO: 10.

In certain embodiments, the promoter comprises at least 400 polynucleotides.

In certain embodiments, the promoter comprises nucleotides 1-538 of SEQ ID NO: 11, nucleotides 127-538 of SEQ ID NO: 11, nucleotides 113,139-113,550 of GenBank Accession No. KT887224, or nucleotides 113,013-113,550 of GenBank Accession No. KT887224.

In certain embodiments, the coding sequence is partially or fully codon-optimized for expression in a mammalian host cell.

In certain embodiments, the most 3′ 300-350 nucleotides of the coding sequence are codon-optimized for expression in the mammalian host cell.

In certain embodiments, the polyadenylation site is a bovine growth hormone (bGH) polyadenylation site.

In certain embodiments, the coding sequence for the ICP27 comprises a mutation that reduces inhibition of host cell pre-mRNA splicing, while permitting HSV late gene expression.

In certain embodiments, the mutation is vBS3.3 double mutation, vBS4.3 double mutation, or vBS5.3 double mutation.

Another aspect of the invention provides a host cell comprising the recombinant vector of the invention, wherein the host cell is capable of expressing the ICP27 or the functional equivalent thereof.

In a related aspect, the invention provides viral production/packaging cell line expressing (e.g., constitutively or inducibly expressing) a functional ICP27 protein, wherein the coding sequence for the functional ICP27 protein has little (e.g., up to 10, 5, 3, 2, 1 bp overlap) or no sequence overlap with the subject rHSV vector having a complete ICP27 gene deletion.

In certain embodiments, wherein coding sequence for the functional ICP27 protein has a codon optimized region at the 3′ end of the coding sequence to minimize sequence homology to wild-type ICP27 coding sequence in the same region. For example, the wildly used d27-1 HSV-1 vector contains a portion of the undeleted ICP27 gene at the 3′ end of the deletion, which undeleted ICP27 gene sequence may overlap with the ICP27 coding sequence in the subject host cell/viral packaging cell line/viral production cell line. By taking advantage of the redundant genetic code, the coding sequence for the subject functional ICP27 in this overlap region can be codon optimized to preserve the encoded amino acid sequences, yet reducing sequence homology at the nucleic acid level to 66% or lower in this region to discourage recombination.

In certain embodiments, the recombinant vector is stably integrated into the host cell genome.

In certain embodiments, the host cell is derived from a vertebrate, such as human, monkey, bovine, porcine, equine and other equids, canine, feline, ovine, goat, murine, rat, rabbit, mink, opossum, camel and other cameloids, chicken and other avian, armadillo, frogs, reptiles, or derived from an insect cell. (Representative cells include BHK cells, Vero cells, HEK293 cells, and others.

Another aspect of the invention provides a method of propagating/amplifying/producing the recombinant replication-defective virus of the invention, the method comprising infecting the host cell of the invention with the recombinant replication-defective virus of the invention.

In certain embodiments, the method further comprises harvesting the recombinant replication-defective virus of the invention from the infected host cell of the invention.

In certain embodiments, there is no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, or 2 bp sequence overlap between the recombinant replication-defective virus of the invention, and the coding sequence for the ICP27 or the functional equivalent thereof.

Another aspect of the invention provides a method of producing a recombinant Adeno-Associated Virus (rAAV) comprising a gene of interest (GOI) coding sequence flanked by AAV ITR sequences, the method comprising co-infecting a production host cell with a first recombinant replication-defective virus of the invention comprising a coding sequence for AAV Rep and Cap proteins, and a second recombinant replication-defective virus of the invention comprising a gene of interest (GOI) flanked by AAV ITR sequences.

Another aspect of the invention provides a method of producing a recombinant Adeno-Associated Virus (rAAV) comprising a gene of interest (GOI) coding sequence flanked by AAV ITR sequences, the method comprising infecting a production host cell with a recombinant replication-defective virus of the invention comprising a coding sequence for AAV Rep and Cap proteins, wherein the production host cell (1) comprises an integrated AAV pro-virus having the GOI coding sequence flanked by AAV ITR sequences; (2) is transfected by a vector (e.g., plasmid) having the GOI coding sequence flanked by the AAV ITR sequences; or (3) is co-infected with a rAAV having the GOI coding sequence flanked by the AAV ITR sequences.

In certain embodiments, the production cell line is BHK, Vero, or HEK293.

In certain embodiments, the GOI is a functional equivalent of dystrophin (e.g., a dystrophin minigene encoding a functional micro-dystrophin protein).

In certain embodiments, the tropism of the AAV include serotypes such as AAV1, AAV2, AAV6, AAV7, AAV8, or AAV9, AAV10, AAV11, preferably AAV9. In certain embodiments, AAV capsids may be genetically modified, or the capsids are synthetic, designer capsids that enhance tissue specific or physiologic compartments delivery of a GOI to a specific tissue such as muscle, skeletal muscle, cardiac muscle, smooth muscle, and the like. In certain embodiments, tropism of AAV is altered through pseudotyping, or the mixing of a capsid and genome from different viral serotypes, in order to improve transduction efficiency, as well as altered tropism. Exemplary pseudotyped AAV includes AAV2/5 that targets myoblasts, or AAV2/6. In certain embodiments, in-silico-derived sequences are synthesized de novo and characterized for biological properties relevant to clinical applications.

In certain embodiments, the gene of interest (GOI) includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type TIM (MPS TUBA sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a microdystrophin gene.

In certain embodiments, the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272.

In certain embodiments, the microdystrophin gene comprises a coding sequence for R16 and R17 spectrin-like repeats for the full length dystrophin protein (such as one described in U.S. Pat. No. 7,892,824).

In certain embodiments, the microdystrophin gene comprises a coding sequence for the R1, R16, R17, R23, and R24 spectrin-like repeats of the full-length dystrophin protein (such as the microdystrophin gene described in PCT/US2016/013733).

Another aspect of the invention provides a method of treating muscular dystrophy in a subject in need thereof, the method comprising administering to the subject a therapeutically effective amount of a recombinant AAV (rAAV) vector encoding a microdystrophin gene, wherein the rAAV is produced by the method of the invention.

In certain embodiments, the method further comprises producing the rAAV by the method of the invention, prior to administering to the subject the rAAV.

Another aspect of the invention provides a method of making a recombinant replication-defective virus derived from the Herpesvirales order, wherein the virus is characterized by a deletion in a gene encoding ICP27, or a functional equivalent gene thereof, wherein the deletion is at least 1,200 bps in length and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp of the most 3′-end of the gene encoding ICP27 (e.g., SEQ ID NO: 11), or the functional equivalent gene thereof, the method comprising creating the deletion of the gene encoding ICP27 or the functional equivalent gene thereof by homologous recombination in a suitable host cell.

In certain embodiments, the homologous recombination is carried out by using a bacterial artificial chromosome (BAC) comprising the genome of the virus derived from the Herpesvirales order (e.g., HSV genome) having the gene encoding ICP27 or the functional equivalent gene thereof.

In certain embodiments, the host cell is an E. coli, or a eukaryotic cell such as a yeast, an insect cell (e.g., SF9), or a mammalian cell. The mammalian cell may be a Vero cell, a baby hamster kidney (BHK) cell, a HeLa cell, a human lung fibroblast MRC-5, a human foreskin fibroblast (HFF), a human embryonic lung fibroblast (HELF), a Madin-Darby canine Kidney cell (MDCK), a Madin-Darby bovine kidney cell (MDBK), or others.

Another aspect of the invention provides a method of generating an ICP27-deleted HSV vector comprising either an AAV rep/cap expression cassette or a gene-of-interest (GOI, such as a dystrophin minigene) flanked by AAV ITR sequences, at the TK locus of the HSV vector, the method comprising: (a) inserting a galK selection marker into either an AAV rep/cap expression cassette or a GOI flanked by AAV ITR sequences, on a donor DNA, via homologous recombination, to generate a galK-labeled AAV rep/cap expression cassette, or a galK-labeled GOI, respectively; (2) inserting the galK-labeled AAV rep/cap expression cassette or the galK-labeled GOI, respectively, into the TK locus of the ICP27-deleted HSV vector via homologous recombination and galK positive selection; and, (3) removing the galK selection marker in the galK-labeled AAV rep/cap expression cassette or the galK-labeled GOI, respectively, from the ICP27-deleted HSV vector via homologous recombination and galK negative selection.

It should be understood that any one embodiment of the invention, including those only described in the Examples, claims, or one of the subsections, can be combined with any other one or more embodiments, unless improper or expressly disclaimed.

### BRIEF DESCRIPTION OF THE DRAWINGS

FIG. 1. BHK21 cells were infected with the d27-1 rHSV vector at a MOI of 0.1, and then transfected with 2.5 μg of corresponding plasmids bearing a polynucleotide sequence of either SEQ ID NO: 1, SEQ ID NO: 2, SEQ ID NO: 3, SEQ ID NO: 4, SEQ ID NO: 5, or an unrelated GFP plasmid control sequence. 24 hours post transfection, cells were washed with PBS, and new medium was added to each well. 72 hours post-infection, cell supernatants were collected from each well and rHSV titers were determined by plaque assay on V27 cell monolayers. LoD—limit of detection.

FIG. 2. BHK-27 clones were infected with the d27-1 rHSV vector at an MOI of 0.1. 72 hours post-infection, cell supernatants were collected from each well and rHSV titers were determined by plaque assay on V27 cell monolayers. LoD—limit of detection.

FIGS. 3 and 4 show robust production of rHSV virus with the complete 2.1-kb (2,077 bp) deletion of the ICP27 (UL54) gene without rcHSV contamination, based on plaque assay as described in Example 1 and Example 8. It is apparent that the subject rHSV vector (Clone 3 in FIG. 3 and Clone 4 in FIG. 4) with complete deletion of the ICP27 gene produced high titer rHSV, as evidenced by the numerous plaques on V27 cells, with essentially no contaminating rcHSV, as evidenced by the absence of plaque on Vero cells.

FIG. 5 shows Vero MW75 subclones top for the Δ27HSV virus DDPCR titers.

FIG. 6 shows Δ27HSV virus plaque titers (pfu/mL) for V75 subclones #4, #20 and #24.

FIG. 7 shows higher titers of the subject HSV vectors with complete ICP27 deletion (SLB27) compared to control HSV vectors with incomplete ICP27 deletion (Δ27HSV) when propagated in the subject V75.4 cells.

FIG. 8 shows unexpected syncytial plaque formation in P6 of almost all tested V75.4 cultures infected with the subject HSV vectors with complete ICP27 deletion, while no syncytial plaque formation was observed in the traditional HSV vector d27-1 with partial ICP27 deletion.

FIG. 9 shows higher AAV9-Dys titers with the subject vectors (SLB27) compared to control HSV vectors (Δ27HSV) in BHK production cells.

### DETAILED DESCRIPTION OF THE INVENTION

### 1. Overview

The current d27-1 rHSV-V27-based vector-host cell system has 815 nucleotides overlap between the sequences in d27-1 virus and HSV-1 sequences integrated in complementing V27 cells. Similar or larger size of the overlap also exists within the other ICP27-deleted viruses and similar complementing cells, i.e., 2-2 cells or B130 cells. This 815 nucleotides or larger overlap enables the homologous recombination between the sequences in ICP27-deleted viruses and HSV-1 sequences integrated in ICP27 complementing cells, resulting in the appearance of wild type-like replication-competent contaminating viruses in the ICP27-deleted virus stocks. These stocks then grow also on non-complementing cells where propagation of ICP27-deleted viruses should be restricted. This is observed by increased virus stocks cytotoxicity and generation of viral plaques on non-complementing cells. These undesirable affects can be alleviated by the rHSV vectors and methods of the invention, by removing the regions creating these overlaps.

Applicant has designed multiple DNA coding sequences encoding ICP27 expression cassettes, for the generation of new ICP27-complementing cell lines useful for the growth and propagation of HSV-1 ICP27 deletion viral mutants. Such expression cassettes can be used in adherent Vero cells, adherent BHK cells, as well as serum-free suspension adapted BHK cell lines, or any other cell line that supports replication of HSV. When these production cells are in use for rHSV production, there will be significantly lower (if not zero) probability of generating replication-competent rcHSV, when compared to propagating rHSV in the currently used d27-1 rHSV-V27-based vector-host cell system. While not wishing to be bound by any particular theory, it is believed that the present invention is partly based on the smaller (or no) sequence homologous region or overlap between the viral genome and the integrated ICP27 gene present in the cellular genome of the ICP27-complementing cells lines, such as new adherent Vero or serum-free suspension adapted BHK cell lines.

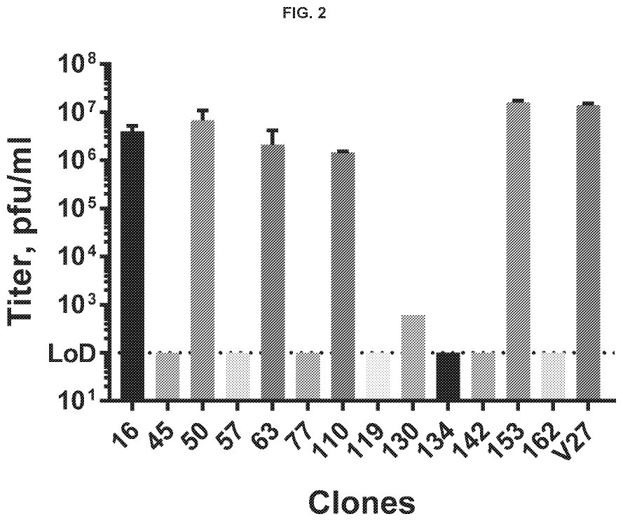

BHK cells that were stably transfected by ICP27 cassette-bearing plasmids with sequences such as SEQ ID NOs: 1, 2, 3, 5, 6, 7, 8, or 9 were named by the isolated clones, such as BHK153 shown in FIG. 2.

In certain embodiments, the rHSV vectors of the invention have the largest ICP27 deletion (˜2 kb deletion) compared to the currently used ICP27-deletion in d27-1 vectors (˜1.6 kb deletion). All analyzed ICP27 expression constructs were able to support replication of rHSV with similar efficiency. Thus, the subject rHSV vectors with ˜2 kb deletion in the ICP27 gene represent a new rHSV production system, which can be used to produce rHSV free of replication-competent HSV (rcHSV).

In particular, Applicant has designed DNA sequences for generating rHSV-1 genome with the entire 2,077-bp UL54 gene deletion. This is currently the largest and complete deletion of the ICP27-encoding UL54 gene. A new replication-defective virus (e.g., replication-defective rHSV-1) encompassing such larger ICP27 deletion will have lower probability of generating replication-competent rcHSV, partly because of the smaller sequence overlap between the new viral genome and any integrated ICP27 gene present in the cellular genome of the current ICP27-complementing cell lines (e.g., V27, 2-2, B130 cells, etc.).

Propagation of a new replication-defective virus (e.g., rHSV-1) harboring such larger ICP27 deletion (e.g., a complete deletion of the ICP27-encoding UL54 gene), in a new adherent Vero cell or serum-free suspension adapted BHK cell lines, which will have no overlap between their ICP27 gene integrated in their cellular genome and viral genome of the rHSV with complete UL54 gene deletion, will enable a production of rHSV stock free of the replication-competent rcHSV virus. Such rcHSV-free rHSV stock can be very useful for large scale production of rAAV, which in turn can be used in gene therapy, for expression of therapeutic proteins (peptides, enzymes, antibodies, etc.), oligonucleotides (i.e., shRNAs, miRNAs), and gene editing and silencing tools (CRISPR-Cas, TALEN, shRNA, miRNA and others), etc.

With the general principle of the invention set forth herein the sections below provides further detailed description for the various aspects of the invention. It should be understood that any embodiment of the invention can be combined with any one or more additional embodiments of the invention, including those embodiments described in different sections of the application, and those described only in the examples, drawings, or claims.

### 2. Recombinant Replication-Defective Virus

In one aspect, the invention described herein provides a recombinant replication-defective virus derived from the Herpesvirales order, wherein the virus is characterized by a deletion in a gene encoding ICP27, or a functional equivalent gene thereof, wherein said deletion is at least 1,200 bps in length and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp of the most 3′-end of said gene encoding ICP27 (e.g., SEQ ID NO: 11), or the functional equivalent gene thereof.

In certain embodiments, the recombinant replication-defective virus (e.g., HSV) is not a clinical strain/non-laboratory strain of HSV.

In certain embodiments, the recombinant replication-defective virus (e.g., HSV) is a laboratory strain of HSV, such as KOS, KOS 1.1, KOS 1.1A, KOS63, KOS79, McKrae, Stain 17, F17, and McIntyre.

As used herein, “clinical strain” and “non-laboratory strain” are used interchangeably herein to refer to viral strains that have been relatively recently or freshly isolated from human or a non-human animal. One key distinction between a laboratory and non-laboratory strain is that laboratory strains have been maintained for long periods (e.g., years in some cases), in culture or serial passage (excluding time spent on storage after freezing down). A laboratory strain that has been through many generations of serial passage in culture may have accumulated mutations that favor rapid replication and growth in culture, but may also have lost certain properties useful for practical applications, such as maintenance of the capacity to travel along axons.

In certain embodiments, a viral vector (e.g., HSV vector) of the invention is derived from a virus strain that has undergone more than three years in culture since isolation of its unmodified clinical precursor strain from its host. The time in culture is time actually spent in culture, excluding storage time after freezing down.

In certain embodiments, a viral vector (e.g., HSV vector) of the invention is derived from a virus strain that has undergone more than 1,000 cycles of serial passage since isolation of its unmodified clinical precursor strain from its host.

Because of the deletion, the recombinant replication-defective virus of the invention is replication defective in the absence of ICP27 protein or a functional equivalent provided in trans by a host cell.

The ICP27 (Infected cell protein 27) gene in human herpesvirus 1 (HHV-1) (Human herpes simplex virus 1) encodes a 512-amino acid protein (UniProtKB-Q3MU88 (Q3MU88_HHV1), incorporated herein by reference). It is also known as mRNA export factor, Immediate-early protein IE63, VMW63, and UL54. A sequence of the ICP27 gene is provided in SEQ ID NO: 11, including the native promoter sequence from HSV-1.

The subject recombinant replication-defective virus can be derived from any virus of the Herpesvirales order, which virus may carry a functional equivalent gene of ICP27 from HSV-1. The deletion of the ICP27 gene or its functional equivalent is at least 1,200 bps in length, and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp (i.e., leave nothing) of the most 3′-end of said gene encoding ICP27 (e.g., SEQ ID NO: 11), or the functional equivalent gene thereof.

The genus Herpesvirus was established in 1971 in the first report of the International Committee on Taxonomy of Viruses (ICTV). This genus consisted of 23 viruses and 4 groups of viruses. In the second ICTV report in 1976, this genus was elevated to family level—the Herpetoviridae. Because of possible confusion with viruses derived from reptiles, this name was changed in the third report in 1979 to Herpesviridae. In this report, the family Herpesviridae was divided into 3 subfamilies (Alphaherpesvirinae, Betaherpesvirinae and Gammaherpesvirinae) and 5 unnamed genera: 21 viruses were listed. In 2009, the family Herpesviridae was elevated to the order Herpesvirales. This elevation was necessitated by the discovery that the herpesviruses of fish and molluscs were only distantly related to those of birds and mammals. Two new families were created—the family Alloherpesviridae which incorporates bony fish and frog viruses and the family Malacoherpesviridae which contains those of molluscs.

The functional equivalent genes from known viruses of the Herpesvirales order, including those from the various named families, subfamilies, genus and species, can be readily obtained from public or proprietary databases, such as GenBank, UniPro, EMBL, etc., using the human HSV-1 ICP27 polynucleotide sequence (such as SEQ ID NO: 11) as a query. Thus these sequences are not described herein, but are otherwise incorporated herein by reference.

In certain embodiments, the most 3′-end of the ICP27 gene or functional equivalent gene thereof is defined by the nucleotide immediately 5′ to the next gene on the respective viral genome (e.g., the first nucleotide of the promoter of the “next” gene 3′ to the ICP27 gene or equivalent).

In certain embodiments, the most 3′-end of the ICP27 gene or functional equivalent gene thereof is defined by the stop codon of ICP27 or the functional equivalent gene thereof, including the stop codon itself.

In certain embodiments, the gene encoding ICP27 has the polynucleotide sequence of SEQ ID NO: 11.

In certain embodiments, the deletion is at least 1,300 bp, 1,400 bp, 1,500 bp, 1,600 bp, 1,700 bp, 1,800 bp, 1,900 bp, 2,000 bp, 2,100 by or more.

In certain embodiments, the deletion comprises, consisting essentially of, or consisting of the entire coding sequence (or ORF) of the gene encoding ICP27 or the functional equivalent gene thereof.

In certain embodiments, the deletion further comprises the entire promoter region of the gene encoding ICP27 or the functional equivalent gene thereof, or a portion (e.g., the most 3′ about 400 nucleotides) of the promoter region.

In certain embodiments, the virus is derived from the Alloherpesviridae family or the Malacoherpesviridae family.

In certain embodiments, the virus is derived from the Herpesviridae family, such as the Alphaherpesvirinae subfamily, the Betaherpesvirinae subfamily, or the Gammaherpesvirinae subfamily.

In certain embodiments, the virus is derived from HHV-1 (Herpes Simplex Virus-1 or HSV-1), HHV-2 (Herpes Simplex Virus-2 or HSV-2), HHV-3 (Varicella Zoster Virus or VZV), HHV-4 (Epstein-Barr Virus or EBV), HHV-5 (Cytomegalovirus or CMV), HHV-6A/HHV-6B (Roseolovirus, Herpes Lymphotropic Virus), HHV-7, or HHV-8 (Kaposi's Sarcoma-Associated Herpesvirus or KSHV).

In certain embodiments, the virus is derived from Cercopithecine herpesvirus-1 (CeHV-1) or Murid Herpesvirus 68 (MHV-68 or MuHV-4).

In certain embodiments, the virus is derived from porcine Alpha-herpesviruses, including pseudorabies virus (PRV).

In certain embodiments, the virus is derived from the Simplexvirus genus, such as Ateline herpesvirus 1, spider monkey herpesvirus, Porcine herpesviruses, Bovine herpesvirus 2, Cercopithecine herpesvirus 1 (Herpes B virus), Fruit bat alphaherpesvirus 1, Leporid herpesvirus 4, Macacine herpesvirus 1, Macropodid herpesvirus 2, and Papiine herpesvirus 2.

In certain embodiments, the virus is derived from the Varicellovirus genus, such as Bovine herpesvirus 1, Bovine herpesvirus 5, Bubaline herpesvirus 1, Caprine herpesvirus 1, Canine herpesvirus 1, Cercopithecine herpesvirus 9, Cervid herpesvirus 1, Cervid herpesvirus 2, Elk herpesvirus 1, Equine herpesvirus 1, Equine herpesvirus 3, Equine herpesvirus 4, Equine herpesvirus 8, Equine herpesvirus 9, Feline herpesvirus 1, and Suid herpesvirus 1.

In certain embodiments, the virus is derived from the Mardivirus genus, such as Anatid herpesvirus 1, Columbiform herpesvirus 1, Gallid herpesvirus 2, Gallid herpesvirus 3 (GaHV-3 or MDV-2), Meleagrid herpesvirus 1 (HVT), and Peacock herpesvirus 1.

In certain embodiments, the virus is derived from the Litovirus genus, such as Gallid herpesvirus 1, and Psittacid herpesvirus 1.

In certain embodiments, the virus is derived from a reptilian Alphaherpesvirus, such as Caretta caretta herpesvirus, Chelonid herpesvirus 1, Chelonid herpesvirus 2, Chelonid herpesvirus 3, Chelonid herpesvirus 4, Chelonia mydas herpesvirus, Coober herpesvirus, Emydid herpesvirus 1, Emydid herpesvirus 2, Fibropapilloma associated herpes virus, Gerrhosaurid herpesvirus 1, Gerrhosaurid herpesvirus 2, Gerrhosaurid herpesvirus 3, Glyptemis herpesvirus 1, Glyptemys herpesvirus 2, Iguanid herpesvirus 1, Iguanid herpesvirus 2, Loggerhead orocutaneous herpesvirus, Lung-eye-trachea associated herpesvirus, Pelomedusid herpesvirus 1, Red eared slider herpes virus, Terrapene herpesvirus 1, Terrapene herpesvirus 2, Testudinid herpesvirus 1, Testudinid herpesvirus 2, Testudinid herpesvirus 3, Testudinid herpesvirus 4, and Varanid herpesvirus 1.

In certain embodiments, the virus is derived from the Rhadinovirus genus, such as Alcelaphine herpesvirus 1, Alcelaphine herpesvirus 2, Ateline herpesvirus 2, Bovine herpesvirus 4, Cercopithecine herpesvirus 17, Equine herpesvirus 2, Equine herpesvirus 5, Equine herpesvirus 7, Japanese macaque rhadinovirus, Leporid herpesvirus 1, and Murid herpesvirus 4 (Murine gammaherpesvirus-68 or MHV-68).

In certain embodiments, the virus is a strain of HSV-1, such as KOS, KOS 1.1, KOS 1.1A, KOS63, KOS79, McKrae, Stain 17, F17, McIntyre, or others.

In certain embodiments, the functional equivalent gene thereof is ORF57 of KSHV, Mta/SM/EB2 of EBV, UL69 of human CMV, or other equivalent genes in any viruses from the Herpesvirales order.

In certain embodiments, the virus further comprises a coding sequence for AAV Rep and Cap proteins, and/or a gene of interest (GOI) flanked by AAV ITR sequences.

In certain embodiments, the coding sequence for the AAV Rep and Cap proteins, and/or the gene of interest (GOI) flanked by AAV ITR sequences is integrated into or replaces a non-essential gene of the virus (e.g., not required for viral replication and not required for viral packaging). Exemplary such non-essential genes include the TK gene, and most of the other about 50% of the viral genome.

Another aspect of the invention provides a method of propagating/amplifying/producing the recombinant replication-defective virus of the invention, the method comprising infecting the subject host cell expressing a complementary/functional ICP27 gene or a functional equivalent thereof (see below), with the subject recombinant replication-defective virus.

In certain embodiments, the method further comprises harvesting the recombinant replication-defective virus from the infected host cell.

In certain embodiments, there is no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, or 2 bp sequence overlap between the subject recombinant replication-defective virus, and the coding sequence for the ICP27 or the functional equivalent thereof which may be integrated into the host cell genome.

The subject recombinant replication-defective virus derived from the Herpesvirales order can be made or constructed using conventional molecular biology techniques, such as homologous recombination. For example, to delete the native ICP27 gene or coding sequence from a wild-type strain of HSV (or a functional equivalent gene from a virus of the Herpesvirales order), the genome of the wild-type virus can be inserted into a suitable vector, such as a bacterial artificial chromosome (BAC) or a yeast artificial chromosome (YAC). Homologous recombination can then be carried out in a suitable host cell, such as an E. coli, a yeast, an insect cell (e.g., SF9), or a mammalian cell, to delete the target gene (i.e., the ICP27 gene or its functional equivalent). This can be done by, for example, introducing into the same host cell a linearized plasmid carrying homologous regions that flank the target gene (i.e., ICP27 or functional equivalent thereof).

Suitable mammalian host cell includes: a Vero cell, a baby hamster kidney (BHK) cell, a HeLa cell, a human lung fibroblast MRC-5, a human foreskin fibroblast (HFF), a human embryonic lung fibroblast (HELF), a Madin-Darby canine Kidney cell (MDCK), a Madin-Darby bovine kidney cell (MDBK), or any other suitable mammalian cells.

Thus another aspect of the invention provides a method of making a recombinant replication-defective virus derived from the Herpesvirales order, wherein the virus is characterized by a deletion in a gene encoding ICP27, or a functional equivalent gene thereof, wherein the deletion is at least 1,200 bps in length and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp of the most 3′-end of the gene encoding ICP27 (e.g., SEQ ID NO: 11), or the functional equivalent thereof, the method comprising creating the deletion of the gene encoding ICP27 or functional equivalent thereof by homologous recombination in a suitable host cell.

In certain embodiments, the homologous recombination is carried out by using a bacterial artificial chromosome (BAC) comprising the genome of the virus derived from the Herpesvirales order (e.g., HSV genome) having the gene encoding ICP27 or the functional equivalent thereof.

In certain embodiments, the cell for amplification of the subject rHSV DNA can be chosen from a large group of cell types, such as an E. coli, or a eukaryotic cell such as a yeast, an insect cell (e.g., SF9), or a mammalian cell. Propagation of the rHSV virus can be conducted in a mammalian cell, e.g., a Vero cell, a baby hamster kidney (BHK) cell, a HeLa cell, a human lung fibroblast MRC-5, a human foreskin fibroblast (HFF), a human embryonic lung fibroblast (HELF), a Madin-Darby canine Kidney cell (MDCK), a Madin-Darby bovine kidney cell (MDBK), or others.

3. Gene of Interest (GOI) in rAAV and Treatable Diseases

The recombinant replication-defective virus of the invention, such as rHSV vectors of the invention, can be used for large scale production of recombinant AAV vectors carrying a gene of interest (GOI). The GOI can be any gene or coding sequence within the packaging capacity of the AAV, e.g., about 4-5 kb, or about 4.7 kb including the ITR sequences, or about 4.4 kb without accounting for the ITR sequences.

In certain embodiments, the rAAV carrying the GOI can be used in gene therapy to treat a disease or condition caused by lacking of function of an endogenous gene in the host, such as a defective version of the GOI.

As used herein, “gene of interest” or GOI generally refers to a nucleic acid or polynucleotide sequence, such as a gene, an open reading frame (ORF), or a coding sequence for protein or RNA such as siRNA. However, in certain circumstances or context, the term GOI also loosely refers to a protein (encoded by the GOI), or a disease or indication that can be remedied by the GOI, or a disease or indication can be (but is not necessarily) caused by loss of function of the GOI.

For example, the gene GALGT2 encodes the protein GalNAc transferase (β-1,4-N-acetylgalactosamine galactosyltransferase), which is an enzyme that transfers a complex sugar molecule onto a few specific proteins, including dystroglycan. Under normal circumstances, GalNAc transferase is found only at the neuromuscular junction (NMJ), where some components of the dystroglycan-associated protein complex are different than elsewhere in muscle. Importantly, at the NMJ, utrophin is present instead of dystrophin. In the mdx mouse model of muscular dystrophy, viral gene transfer of GALGT2 results in expression of GalNAc transferase across the entire muscle membrane, instead of just at the normal expression domain of the NMJ, as well as upregulation of utrophin across the entire muscle fiber. In the mdx mouse, this expression can correct muscle functional deficits to the same degree as does microdystrophin gene expression. Furthermore, overexpression of GALGT2 corrects muscle pathology in mouse models of other muscular dystrophies, including LGMD2A and congenital muscular dystrophy (MDC1A). Thus GALGT2 is a GOI for treating muscular dystrophy such as DMD, BMD, LGMD2A and MDC1A, even though GALGT2 is not necessarily defective per se in the patient in need of treatment.

In another example, Sarcolipin (SLN) inhibits the sarco/endoplasmic reticulum (SR) Ca²⁺ ATPase (SERCA), and is abnormally elevated in the muscle of DMD patients and animal models such as the mdx mouse model of DMD. Reducing SLN levels by AAV9-mediated RNA interference ameliorates dystrophic pathology in the severe dystrophin/utrophin double mutant (mdx:utr^{-/-}) mouse model of DMD, including attenuation of muscle pathology and improvement of diaphragm, skeletal muscle and cardiac function. Thus the coding sequence for SLN RNAi is a GOI that remedies DMD.

Thus the GOI can be a gene (or protein) that, when expressed, replaces a mutated, damaged, or inactive gene or protein. The GOI can be a gene (or protein) that, when expressed, assists an already functioning process that requires modification for therapy in a disease, disorder, or dysfunction. The GOI can be a gene (or protein) that, when expressed, assists a dysfunctional process that requires modification for therapy in a disease, disorder, or dysfunction. A GOI nucleic acid sequence can be DNA, RNA, or synthetic nucleic acid molecule. The GOI can be a protein, an enzyme, a structural protein, a functional protein, or an adaptable protein based on cell function(s). The GOI can provide therapeutic benefit or a treatment modality for a disease, disorder, or dysfunction.

In certain embodiments, the GOI may be CRISPR-Cas9, Cas 13, TALEN, or other genetic based gene editing protein that are required for intracellular delivery for their intended activity.

Any and all GOIs as used herein may require codon optimization for enhanced expression and activity via known computer based algorithms.

Thus the rAAV that may be produced by using the subject viral vectors (e.g., rHSV vectors) and the complementary cells (which supply the ICP27 gene product in trans), may encode a gene of interest (GOI) useful for, e.g., gene therapy to treat a disease or condition. Representative (non-limiting) gene of interest (GOI) may include: a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS TUBA sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a microdystrophin gene.

In certain embodiments, the microdystrophin gene is any one described in the following patents: U.S. Pat. Nos. 7,906,111; 7,001,761; 7,510,867; 6,869,777; 8,501,920; 7,892,824; PCT/US2016/013733; U.S. Pat. No. 10,166,272 (all incorporated herein by reference). In certain embodiments, the microdystrophin gene is capable of being packaged into a rAAV virion, e.g., no more than about 4.7 kb in size.

In certain embodiments, the microdystrophin gene contains within its coding sequence spectrin-like repeats R16 and R17 that are capable of restoring nitric oxide synthase (nNOS) activity to the sarcolemma (such as those described in U.S. Pat. No. 7,892,824).

In certain embodiments, the microdystrophin gene comprises a coding sequence for the R1, R16, R17, R23, and R24 spectrin-like repeats (i.e., SR1, SR16, SR17, SR23, and SR24, respectively) of the full-length dystrophin protein, such as one described in PCT/US2016/013733 (incorporated herein by reference). In certain embodiments, the microdystrophin gene does not encode any other spectrin repeats of the full-length dystrophin protein, other than SR1, SR16, SR17, SR23, and SR24.

Diseases or conditions having a potential to benefit from the rAAV produced by the rHSV-based system include: Huntington's disease, X-linked myotubular myopathy (XLMTM), Acid maltase deficiency (e.g., Pompe disease), Spinal Muscular Atrophy (SMA), Myasthenia Gravis (MG), Amyotrophic lateral sclerosis (ALS), Friedreich's ataxia, Mitochondrial myopathy, Muscular dystrophies (Duchenne's muscular dystrophy, Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), Mucopolysaccharidoses (MPS), Metachromatic leukodystrophy (MLD), Batten Disease, Rett Syndrome, Krabbe Disease, Canavan disease, X-Linked Retinoschisis, Achromatopsia (CNGB3 and CNGA3), X-Linked Retinitis Pigmentosa, Age-Related Macular Degeneration, neovascularized macular degeneration, Pompe, Fabry's disease, MPS I, II, IIIA, IIIB, Gaucher's disease, Dannon Disease, AlAt Deficiency, Friedreich ataxia, Wilson's Disease, Batten Disease (CLN1, CLN3, CLN6, CLN8), Wolman Disease, Tay-Sachs, Niemann-Lick Type C, CDKL5 deficiency Disorder, B-thalassemia, Sickle cell disease.

In certain embodiments, diseases or conditions having a potential to benefit from the rAAV produced by the rHSV-based system may include: Becker muscular dystrophy (BMD), Congenital muscular dystrophies (CMD), Bethlem CMD, Fukuyama CMD, Muscle-eye-brain diseases (MEBs), Rigid spine syndromes, Ullrich CMD, Walker-Warburg syndromes (WWS), Duchenne muscular dystrophy (DMD), Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), Motor neuron diseases including ALS (amyotrophic lateral sclerosis), Spinal-bulbar muscular atrophy (SBMA), Spinal muscular atrophy (SMA).

In certain embodiments, diseases or conditions having a potential to benefit from the rAAV produced by the rHSV-based system may include ion channel diseases, which are typically marked by muscular weakness, absent muscle tone, or episodic muscle paralysis. They include Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia.

In certain embodiments, diseases or conditions having a potential to benefit from the rAAV produced by the rHSV-based system may include mitochondrial diseases, which occur when structures that produce energy for a cell malfunction. Such diseases include: Friedreich's ataxia (FA), Mitochondrial myopathies, Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO).

In certain embodiments, diseases or conditions having a potential to benefit from the rAAV produced by the rHSV-based system may include myopathies, which is a disease of muscle in which the muscle fibers do not function properly, resulting in muscular weakness. Myopathies include: Cap myopathies, Centronuclear myopathies, Congenital myopathies with fiber type disproportion, Core myopathies, Central core disease, Multiminicore myopathies, Myosin storage myopathies, Myotubular myopathy, Nemaline myopathies, Distal myopathies, GNE myopathy/Nonaka myopathy/hereditary inclusion-body myopathy (HIBM), Laing distal myopathy, Markesberg-Griggs late-onset distal myopathy, Miyoshi myopathy, Udd myopathy/tibial muscular dystrophy, Vocal cord and pharyngeal distal myopathy, Welander distal myopathy, Endocrine myopathies, Hyperthyroid myopathy, Hypothyroid myopathy, Inflammatory myopathies, Dermatomyositis, Inclusion-body myositis, Polymyositis, Metabolic myopathies, Acid maltase deficiency (AMD, Pompe disease), Carnitine deficiency, Carnitine palmityl transferase deficiency, Debrancher enzyme deficiency (Cori disease, Forbes disease), Lactate dehydrogenase deficiency, Myoadenylate deaminase deficiency, Phosphofructokinase deficiency (Tarui disease), Phosphoglycerate kinase deficiency, Phosphoglycerate mutase deficiency, Phosphorylase deficiency (McArdle disease), Myofibrillar myopathies (MFM), Scapuloperoneal myopathy.

In certain embodiments, diseases or conditions having a potential to benefit from the rAAV produced by the rHSV-based system may include neuromuscular junction diseases, which result from the destruction, malfunction or absence of one or more key proteins involved in the transmission of signals between muscles and nerves. Such diseases include: Congenital myasthenic syndromes (CMS), Lambert-Eaton myasthenic syndrome (LEMS), Myasthenia gravis (MG).

In certain embodiments, diseases or conditions having a potential to benefit from the rAAV produced by the rHSV-based system may include peripheral nerve diseases, in which the motor and sensory nerves that connect the brain and spinal cord to the rest of the body are affected, causing impaired sensations, movement or other functions. Such diseases include: Charcot-Marie-Tooth disease (CMT), Giant axonal neuropathy (GAN), muscle wasting in cachexia and aging.

### 4. Complementary Recombinant Vectors

The viral vectors of the invention, such as the recombinant HSV vectors of the invention, can be propagated in a suitable host cell that provides the ICP27 function deleted from the subject viral vectors. Such ICP27 function can be provided by the subject complementary recombinant vectors (or “recombinant vector” for short) encoding ICP27. The complementary recombinant vectors of the invention may be integrated into the genome of the host cell. The ICP27 coding sequence may be transcribed from the native promoter in the Herpesvirales genome from which the ICP27 gene originates.

Thus another aspect of the invention provides a (complementary) recombinant vector capable of expressing ICP27 or a functional equivalent thereof in a host cell, the vector comprising: (1) a coding sequence for the ICP27 or the functional equivalent thereof, operatively linked to a promoter capable of directing the transcription of the coding sequence in the host cell; (2) a polyadenylation site 3′ to the coding sequence; and, (3) optionally, one or more multi-cloning site(s); wherein the vector contains no more than 300 bp, 250 bp, 200 bp, 150 bp, 100 bp, 50 bp, 30 bp, 20 bp, 10 bp, 9 bp, 8 bp, 7 bp, or 6 bp consecutive nucleotides of any of the subject recombinant replication-defective virus (e.g., rHSV).

Since the ICP27 protein provided in trans does not need to be 100% as active as the wild-type ICP27, in certain embodiments, the ICP27 has the amino acid sequence of SEQ ID NO: 10, or is at least 60%, 70%, 75%, 80%, 85%, 90%, 95%, 97%, 98%, 99%, 99.2%, 99.4%, 99.6%, or 99.8% identical to SEQ ID NO: 10.

Likewise, the promoter of the ICP27 coding sequence does not need to be the native promoter in the virus from which the ICP27 originates, though in some embodiment, the promoter is the native promoter.

In certain embodiments, the promoter comprises at least about 400 polynucleotides, 450 polynucleotides, 0.500 polynucleotides, or about 550 polynucleotides.

In certain embodiments, the promoter comprises nucleotides 1-538 of SEQ ID NO: 11, or nucleotides 127-538 of SEQ ID NO: 11, or nucleotides 113,139-113,550 of GenBank Accession No. KT887224, or nucleotides 113,013-113,550 of GenBank Accession No. KT887224 (entire sequence incorporated herein by reference).

In certain embodiments, there is no overlap in sequence between the ICP27 coding sequence on the complementary recombinant vectors of the invention, and the deleted ICP27 coding sequence from the subject viral vectors.

In certain embodiments, the ICP27 coding sequence as the complementary DNA in the host cell (useful for the propagation of the subject viral (e.g., rHSV) vectors devoid of ICP27 coding sequence) is partially or fully codon-optimized for translation in a eukaryote or mammalian cell line, such as in BHK cells, Vero cells, or HEK293 cells. For example, in certain embodiments, the most 3′ 300-350 nucleotides of the coding sequence are codon-optimized for expression in the mammalian host cell.

In certain embodiments, the polyadenylation site is a bovine growth hormone (bGH) polyadenylation site.

In certain embodiments, the polyadenylation site or poly(A) signal sequence is from other suitable sources, e.g., synthetic sequences or sequences from other eukaryotic genes or viruses.

In certain embodiments, any minimal or residual overlap between the ICP27 coding sequence on the complementary recombinant vectors of the invention, and the subject recombinant replication-defective viral vector without the deleted ICP27 coding sequence/ORF, is not significant enough to permit or support homologous recombination between the two. For example, there is minimal (if any) residue ICP27 coding sequence and/or promoter sequence on the subject viral vector, such that any overlap in sequence between the subject viral vector and the ICP27 coding sequence (plus any native promoter sequence) is insufficient to lead to homologous recombination.

In certain embodiments, the ICP27 coding sequence as the complementary DNA in the host cell (useful for the propagation of the subject (e.g., rHSV) vectors devoid of ICP27 coding sequence) comprises a mutation that reduce the ICP27 protein's ability to inhibit host pre-mRNA splicing, while still allowing the promotion of late gene expression. Such ICP27 mutation may lead to a greater infectious rAAV yield due at least partly to increased expression of the AAV Rep and Cap proteins.

Exemplary such mutations include the vBS3.3, vBS4.3, and vBS5.3 mutations as described by Soliman et al. (J Virol 71:9188-9197, 1997, incorporated herein by reference). Specifically, Soliman described the use of a temperature sensitive ICP27 allele—LG4—that loses ICP27 activity at the restrictive temperature of 39.5° C., in a genetic screen for intragenic suppressors. Three such intragenic suppressors were identified, namely vBS3.3, vBS4.3, and vBS5.3. The LG4 allele has a single R480H point mutation just N-terminal to the carboxy-terminal zinc finger in wild-type ICP27. The vBS3.3, vBS4.3, and vBS5.3 intragenic suppressor alleles have all retained the original R480H point mutation, but also contain one additional point mutation of V496I, S334L, and V487I, respectively. Thus the vBS3.3 is a double point mutation of R480H and V496I. The vBS4.3 is a double point mutation of R480H and S334L. The vBS5.3 is a double point mutation of R480H and V487I.

### 5. Host Cells

Many different types of eukaryote host cells can be used to propagate the subject recombinant replication-defective viral vectors, provided that such eukaryote host cells are engineered to express ICP27 deleted from the subject recombinant replication-defective viral vectors.

In certain embodiments, the subject host cells comprise the subject complementary recombinant vectors, and are capable of expressing ICP27 to promote replication and packaging of the subject viral vectors.

In certain embodiments, the recombinant vector is stably integrated into the host cell genome.

In certain embodiments, the host cell is derived from a vertebrate, such as human, monkey, bovine, porcine, equine and other equids, canine, feline, ovine, goat, murine, rat, rabbit, mink, opossum, camel and other cameloids, chicken and other avian, armadillo, frog, or reptile, or derived from an insect cell. Human cells include BHK cells, Vero cells, HEK293 cells, etc.

In certain embodiments, the host cell is HEK293 (human embryonic kidney), which can be grown using standard tissue culture media such as DMEM complemented with L-Gln, 5-10% fetal bovine serum (FBS), and 1% penicillin-streptomycin. For growing adherent HEK293 cells, the percentage of FBS can be reduced during rAAV production in order to limit contamination by animal-derived components.

In certain embodiments, the host cell is a Vero cell. Such cells may grow on a solid support, including tissue culture plates, dishes, flasks, bottles, and microcarrier that allows the adherent Vero cells to grow in suspension-like conditions.

In certain embodiments, the host cell is a BHK (baby hamster kidney) cell, such as BHK21. In certain embodiments, the BHK cells are adapted to grow in serum-free suspension.

In certain embodiments, the host cell is a HEK293 cell. In certain embodiments, the HEK293 cell is adapted for growth in serum-free media (such as F17 or Expi293 media) and in suspension, thus is amenable for large scale growth in a bioreactor. See, for example, Grieger et al. (Mol. Ther. 24:287-297, 2016, incorporated herein by reference).

In certain embodiments, the HEK293 cell is a HEK293T cell which expresses SV40 T antigen (the temperature sensitive allele tsA1609) and the neomycin/geneticin-resistance gene.

In certain embodiments, the cell for amplification of the subject rHSV DNA can be chosen from a large group of cell types, such as an E. coli, or a eukaryotic cell such as a yeast, an insect cell (e.g., SF9), or a mammalian cell. Propagation of the rHSV virus can be conducted in a mammalian cell, e.g., a Vero cell, a baby hamster kidney (BHK) cell, a HeLa cell, a human lung fibroblast MRC-5, a human foreskin fibroblast (HFF), a human embryonic lung fibroblast (HELF), a Madin-Darby canine Kidney cell (MDCK), a Madin-Darby bovine kidney cell (MDBK), or others.

In certain embodiments, stocks of viral vectors so propagated can be checked to ensure that no replication competent viral vectors are present. For example, assays used in Example 1 can be used to determine the titer of rHSV, and the presence or absence rcHSV.

In certain embodiments, the viral vectors of the invention may be adapted for use in producing recombinant AAV vectors encoding a gene of interest (GOI), which may be used in gene therapy. See the section entitled “Recombinant AAV Production” below. In such embodiments, one or more rAAV production cell lines may be infected by the subject viral vectors, such as rHSV vector encoding AAV Rep and Cap proteins, and optionally rHSV vector encoding the GOI flanked by AAV ITR sequences.

In certain embodiments, such producer cell line for rAAV production is a HeLa- or A549-derived cell line transfected with a plasmid containing both rep-cap genes of AAV, and an rAAV vector genome (with the GOI) along with a drug selection marker.

In certain embodiments, such producer cell line for rAAV production is a Vero cell.

In certain embodiments, such producer cell line for rAAV production is a BHK cell.

In certain embodiments, such producer cell line for rAAV production is a HEK293 cell.

In certain embodiments, such producer cell line for rAAV production comprises an rAAV provirus that encodes the GOI flanked by the AAV ITR sequences, wherein the rAAV provirus is integrated into the genome of the producer cell line for rAAV production. The GOI can be any one of the GOI described herein useful for gene therapy, such as a dystrophin minigene or a microdystrophin gene described in U.S. Pat. Nos. 7,906,111; 7,001,761; 7,510,867; 6,869,777; 8,501,920; 7,892,824; or U.S. Pat. No. 10,166,272, or in PCT/US2016/013733 (all incorporated herein by reference).

For example, PCT/US2016/013733 (WO2016/115543A2) provides a micro-dystrophin gene operatively connected to a regulatory cassette, wherein the micro-dystrophin gene encodes a protein comprising: an amino-terminal actin-binding domain; a β-dystroglycan binding domain; and a spectrin-like repeat domain, comprising at least four spectrin-like repeats, wherein two of the at least four spectrin-like repeats comprise a neuronal nitric oxide synthase binding domain. In certain embodiments, the at least four spectrin-like repeats include spectrin-like repeat 1 (SR1), spectrin-like repeat 16 (SR16), spectrin-like repeat 17 (SR17), and spectrin-like repeat 24 (SR24). In certain embodiments, the protein encoded by the micro-dystrophin gene further comprises at least a portion of a hinge domain, such as at least one of a Hinge 1 domain, a Hinge 2 domain, a Hinge 3 domain, a Hinge 4 domain, and a hinge-like domain. In certain embodiments, the micro-dystrophin gene comprises, in N- to C-terminal order: a Hinge 1 domain (H1); a spectrin-like repeat 1 (SR1); a spectrin-like repeat 16 (SR16); a spectrin-like repeat 17 (SR17); a spectrin-like repeat 24 (SR24); and a Hinge 4 domain (H4). In certain embodiments, H1 is directly coupled to the SR1. In certain embodiments, SR 1 is directly coupled to SR16. In certain embodiments, SR16 is directly coupled to SR17. In certain embodiments, SR 17 is directly coupled to SR24. In certain embodiments, SR24 is directly coupled to the H4. In certain embodiments, the protein encoded by the micro-dystrophin gene further comprises between SR1 and SR16, in N- to C-terminal order, a spectrin-like repeat 2 (SR2) and a spectrin-like repeat 3 (SR3). In certain embodiments, SR1 is directly coupled to SR2 and SR2 is further coupled to SR3. In certain embodiments, H1 is directly coupled to SR1, SR1 is directly coupled to SR16, SR16 is directly coupled to SR17, SR17 is directly coupled to SR23, SR23 is directly coupled to SR24, and SR24 is directly coupled to H4.

In certain embodiments, the regulatory cassette is selected from the group consisting of a CK8 promoter and a cardiac troponin T (cTnT) promoter. In certain embodiments, the protein encoded by the micro-dystrophin gene has between five spectrin-like repeats and eight spectrin-like repeats. In certain embodiments, the protein encoded by the micro-dystrophin gene has at least 80% or 90% sequence identity to the amino acid sequence of SEQ ID NO: 4 or 5 in WO2016/115543A2 (incorporated herein by reference).

In certain embodiments, rAAV viral vectors so produced can be checked to ensure that no rHSV and no rcHSV viral vectors are present in the rAAV viral stock. For example, assays used in Example 1 can be used to determine the presence or absence rHSV and rcHSV.

### 6. Recombinant AAV Production

The subject recombinant replication-defective viral vectors, especially the subject recombinant HSV vectors, as well as the production cell lines, together form an HSV-based complementation system that can be used for large scale production of recombinant AAV vectors (rAAV) useful for gene therapy.

Recombinant AAV vectors, which can be produced with the subject viral (e.g., rHSV) vectors and production cell lines, typically comprise a gene of interest (GOI) and expression regulators (such as promoters for the GOI) in lieu of the wild-type AAV virus rep and cap open reading frames (ORFs). The AAV rep and cap ORFs, optionally their native promoters p5, p19, and p40, are instead supplied by the subject recombinant HSV vector and/or production cell line. The rep ORF encodes four nonstructural Rep proteins involved in the AAV viral life cycle, and the cap ORF encodes the three structural proteins (i.e., VP1, VP2, and VP3) that form the icosahedral AAV capsid. Typically, the only AAV viral sequences that are retained in the rAAV vector genome are the inverted terminal repeats (ITRs)—the minimal cis-acting elements required for AAV DNA replication and packaging.

The gene of interest (GOI) may include genes useful for gene therapy in treating certain diseases or conditions. Representative (non-limiting) GOI may include a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS IBB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

Suitable microdystrophin genes have been described in the following patents: U.S. Pat. Nos. 7,906,111; 7,001,761; 7,510,867; 6,869,777; 8,501,920; 7,892,824; PCT/US2016/013733; U.S. Pat. No. 10,166,272 (all incorporated herein by reference).

Diseases or conditions having a potential to benefit from the rAAV produced by the subject rHSV-based system include: Huntington's disease, X-linked myotubular myopathy (XLMTM), Acid maltase deficiency (e.g., Pompe disease), Spinal Muscular Atrophy (SMA), Myasthenia Gravis (MG), Amyotrophic lateral sclerosis (ALS), Friedreich's ataxia, Mitochondrial myopathy, Muscular dystrophies (Duchenne's muscular dystrophy, Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), Mucopolysaccharidoses (MPS), Metachromatic leukodystrophy (MLD), Batten Disease, Rett Syndrome, Krabbe Disease, Canavan disease, X-Linked Retinoschisis, Achromatopsia (CNGB3 and CNGA3), X-Linked Retinitis Pigmentosa, Age-Related Macular Degeneration, neovascularized macular degeneration, Pompe, Fabry's disease, MPS I, II, IIIA, IIIB, Gaucher's disease, Dannon Disease, AlAt Deficiency, Friedreich ataxia, Wilson's Disease, Batten Disease (CLN1, CLN3, CLN6, CLN8), Wolman Disease, Tay-Sachs, Niemann-Lick Type C, CDKL5 deficiency Disorder, B-thalassemia, Sickle cell disease, etc.

Being a naturally replication-defective human parvovirus, wild-type AAV integrates its genome site-specifically within the host cell chromosome in the absence of helper assistance for its replication, where it persists indefinitely unless rescued via cellular infection with a helper virus. The introduction of a helper virus into the host cell triggers AAV replication and the generation of progeny virions. In the case of rAAV virions useful for gene therapy, introduction of the helper virus function into a suitable host cell triggers the packaging of the GOI in the rAAV virions, if the requisite rep and cap coding sequences are also supplied in the same system.

In other words, production of recombinant AAV relies entirely on (1) the presence of the AAV rep and cap coding sequences, and (2) the helper virus functions. The subject recombinant (HSV) vector and production cell line can provide both required functionality for rAAV production.

Viruses of the Herpesviridae families, such as HSV, have been shown to provide the essential trans functions for AAV replication (Handa and Carter, J. Biol. Chem. 254:6603-6610, 1979; Buller et al., J. Virol. 40:241-247, 1981). For simplicity, the description herein refers to HSV as a specific example of a virus from the Herpesviridae family that can provide the essential trans functions for AAV replication, and its should be understood that the description generally applies to other virus from the Herpesvirales order, such as the Herpesviridae family.

Replication proteins from viruses of the Herpesvirales order, such as the Herpesviridae family (e.g., HSV), can be used directly by AAV for efficient genome replication and packaging.

In certain embodiments, the subject rHSV can be used with an HSV amplicon-based system to produce rAAV with GOI. According to this embodiment, the AAV rep and cap coding sequences, optionally with their native promoters (p5, p19, and p40), are provided by a so-called pHSV-RC plasmid carrying the HSV origin of replication and packaging signal (e.g., an HSV amplicon). HSV particles carrying the AAV rep and cap genes are generated by transfecting the pHSV-RC plasmid into a suitable host cell, such as a Vero cell, which is infected with the subject rHSV vector with the ICP27 deletion. In this system, the subject rHSV vector with the ICP27 deletion was used as a helper virus to supply the missing trans factors required for HSV amplicon DNA replication and packaging into HSV particles. HSV particles so generated can be further amplified through serial infection passages, by infecting suitable host cells (such as Vero cells) with the HSV particles and the subject rHSV vector with the ICP27 deletion. In certain embodiments, recombinant AAV vectors with a desired GOI is produced by infecting a proviral cell line with such HSV particles having AAV rep and cap genes, wherein the proviral cell line contains the rAAV with the GOI integrated into the genome of the proviral cell line. In certain embodiments, recombinant AAV vectors with a desired GOI is produced by infecting a cell with such HSV particles having AAV rep and cap genes, wherein the cell is transfected with an rAAV plasmid having the GOI flanked by the AAV ITR sequences. In certain embodiments, recombinant AAV vectors with a desired GOI is produced by infecting a cell with such HSV particles having AAV rep and cap genes, wherein the cell is infected with a rAAV having the GOI.

In certain embodiments, the subject rHSV can be used directly to produce rAAV with GOI, by incorporating the AAV rep and cap coding sequences into the subject rHSV vector. According to this embodiment, the AAV rep and cap genes can be inserted into (with or without replacing) a non-essential gene (such as the thymidine kinase (TK) locus) of the subject replication-defective rHSV vector with ICP27 deletion, by, e.g., homologous recombination. The resulting rHSV can be propagated in V27-like cells (such as those derived from Vero cells) of the invention that contain an ICP27 coding sequence not overlapping (or minimally overlapping) with the subject ICP27-deleted rHSV. The resulting rHSV particles can produce the requisite AAV Rep and Cap proteins when used to infect a suitable AAV production cell line (such as BHK cells, Vero cells, or HEK293 cells). In certain embodiments, recombinant AAV vectors with a desired GOI is produced by infecting a proviral cell line with such rHSV particles having AAV rep and cap genes, wherein the proviral cell line contains the rAAV with the GOI integrated into the genome of the proviral cell line. In certain embodiments, recombinant AAV vectors with a desired GOI is produced by infecting a cell with such rHSV particles having AAV rep and cap genes, wherein the cell is also transfected with an rAAV plasmid having the GOI flanked by the AAV ITR sequences. In certain embodiments, recombinant AAV vectors with a desired GOI is produced by infecting a cell with such rHSV particles having AAV rep and cap genes, wherein the cell is also infected with a rAAV having the GOI.

In certain embodiments, the subject rHSV can be used directly to produce rAAV with GOI, by incorporating the AAV rep and cap coding sequences into a first subject rHSV vector, and incorporating AAV ITR-flanked GOI into a second subject rHSV vector. The AAV rep and cap coding sequences, and the AAV ITR-flanked GOI may be inserted into the same (or different) non-essential gene locus on the subject rHSV, such as the thymidine kinase (TK) locus of the subject replication-defective rHSV vector with ICP27 deletion, by, e.g., homologous recombination. The resulting rHSVs can be propagated in V27-like cells (such as those derived from Vero cells) of the invention that contain an ICP27 coding sequence not overlapping (or minimally overlapping) with the subject ICP27-deleted rHSV. The resulting rHSV particles—a first population of rHSV particles carrying the AAV rep and cap coding sequences, and a second population of rHVS particles carrying the AAV ITR-flanked GOI, can be used to co-infect an AAV production cell line, such as BHK cells, Vero cells, or HEK293 cells to produce rAAV virions carrying the GOI. This is a rAAV manufacturing system based solely on HSV infection. rAAV particles carrying the GOI can be produced by first co-infecting a suitable rHSV production cell line (such as Vero cells or BHK cells) with the requisite complementation system (such as the ICP27 coding sequence not overlapping with the subject rHSV) to produce rHSV stock. After rHSV vector recovery and concentration to high titer, rHSV vectors carrying the AAV rep and cap coding sequences, and rHSV vectors carrying the AAV ITR-flanked GOI are used to co-infect a suitable AAV production cell line, such as HEK293 or BHK cells, to produce rAAV vectors carrying the GOI.

Thus in another aspect, the invention provide a method of producing a recombinant Adeno-Associated Virus (rAAV) comprising a gene of interest (GOI) coding sequence flanked by AAV ITR sequences, the method comprising co-infecting a production host cell with a first recombinant replication-defective virus comprising a coding sequence for AAV Rep and Cap proteins, and a second recombinant replication-defective virus comprising a gene of interest (GOI) flanked by AAV ITR sequences.

In a related aspect, the invention provides a method of producing a recombinant Adeno-Associated Virus (rAAV) comprising a gene of interest (GOI) coding sequence flanked by AAV ITR sequences, the method comprising infecting a production host cell with a recombinant replication-defective virus comprising a coding sequence for AAV Rep and Cap proteins, wherein the production host cell (1) comprises an integrated AAV pro-virus having the GOI coding sequence flanked by AAV ITR sequences; (2) is transfected by a vector (e.g., plasmid) having the GOI coding sequence flanked by the AAV ITR sequences; or (3) is co-infected with a rAAV having the GOI coding sequence flanked by the AAV ITR sequences.

In certain embodiments, the production cell line is BHK, Vero, or HEK293.

In certain embodiments, the tropism of the AAV include serotypes such as AAV1, AAV2, AAV6, AAV7, AAV8, or AAV9, AAV10, AAV11, preferably AAV9. In certain embodiments, AAV capsids may be genetically modified, or capsids may be synthetic, designer capsids that enhance tissue specific or physiologic compartments delivery of a GOI to a specific tissue such as muscle, skeletal muscle, cardiac muscle, smooth muscle, and the like, as described (see, e.g., Zinn and Grimm, High-Throughput Dissection of AAV—Host Interactions: The Fast and the Curious, JMB 430(17):2626-2640, 2018; Kotterman and Schaffer, Engineering adeno-associated viruses for clinical gene therapy. Nature Reviews Genetics (2014) 4445-4451, both incorporated herein by reference). Tropism of AAV through pseudotyping, or the mixing of a capsid and genome from different viral serotypes may also be employed. These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, pseudotyped AAV2/5 targets myoblasts (Duan et al., Enhancement of muscle gene delivery with pseudotyped adeno-associated virus type 5 correlated with myoblast differentiation. J Virol 75(16):7662-7671, 2001). Other pseudotyped AAV includes AAV2/6. In certain embodiments, In-silico-derived sequences were synthesized de novo and characterized for biological properties relevant to clinical applications. This effort led to the generation of nine functional putative ancestral AAVs and the identification of Anc80, the predicted ancestor of the widely studied AAV serotypes 1, 2, 8, and 9, as a highly potent in vivo gene therapy vector for targeting liver, muscle, and retina (Zinn et al., In Silico Reconstruction of the Viral Evolutionary Lineage Yields a Potent Gene Therapy Vector, Cell Reports 12(6):1056-1068, 2015); Buning et al., Engineering the AAV capsid to optimize vector—host-interactions, Current Opinion in Pharmacology, 24:94-104, 2015).

In certain embodiments, the tropism of the AAV include skeletal muscle (such as AAV1, AAV6, AAV7, AAV8, or AAV9, preferably AAV9).

In certain embodiments, the gene of interest (GOI) includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS IBB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a functional equivalent of dystrophin (e.g., a dystrophin minigene encoding a functional micro-dystrophin protein).

In certain embodiments, the GOI is a microdystrophin gene.

In certain embodiments, the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272.

In certain embodiments, the microdystrophin gene comprises a coding sequence for R16 and R17 spectrin-like repeats for the full length dystrophin protein (such as one described in U.S. Pat. No. 7,892,824).

In certain embodiments, the microdystrophin gene comprises a coding sequence for the R1, R16, R17, R23, and R24 spectrin-like repeats of the full-length dystrophin protein (such as the microdystrophin gene described in PCT/US2016/013733).

In certain embodiments, the microdystrophin gene does not comprise coding sequence for and spectrin repeats of the full-length dystrophin protein other than the SR1, SR16, SR17, SR23, and SR24 repeats (e.g., in that order).

In certain embodiments, the subject rHSV provides the minimal set of HSV genes required for AAV production, including the HSV core replication machinery—the HSV helicase-primase complex (encoded by UL5, UL8, and UL52), and the single-stranded DNA-binding protein encoded by UL29—as well as other HSV genes including the HSV polymerase UL30, the polymerase accessory factor UL42, and the origin-binding protein UL9.

In certain embodiments, the subject recombinant HSV vector has, in addition to the ICP27 deletion described herein, deletion of one or more further immediate-early (IE) genes encoding infected cell proteins (ICPs), such as ICP0, ICP4, ICP22, and/or ICP47. The production of the subject replication-incompetent rHSV vectors requires adequate complementing cell lines for providing in trans the missing replication and packaging functions of rHSV.

In certain embodiments, in addition to ICP27 deletion, the subject rHSV further lacks gene(s) encoding HSV glycoprotein H (gH). Infectious particles could be generated from such rHSV only from a complementing gH-expressing cell line, thus conferring a further level of safety.

Other than the essential feature relating to the ICP27 deletion described herein, the subject rHSV vector may retain most of the wild-type HSV genome, or have more than 50% of the wild-type HSV genome encoding nonessential gene products deleted without jeopardizing viral amplification. The subject rHSV vector may also comprise two cis-acting elements required for HSV replication and packaging—the origin of replication (oriS), and the packaging signal (sequence a or pac).

In certain embodiments, the subject rHSV and/or rAAV vectors are produced in in vitro culture conditions, such as in bioreactors (e.g., 0.5L, 1L, 2L, 3L, 5L, 10L, 20L, 50L, 100L, 250L, 500L, or 1,000L working volume bioreactors), such as a CelliGen Plus packed-bed bioreactor (New Brunswick Scientific) for fed-batch vector production for 3 days post infection.

In certain embodiments, the subject rHSV vectors are produced as in vitro culture on adherence-dependent cell lines, such as Vero and Vero-derived cell lines, that rely on a solid support. In certain embodiments, the solid support is a tissue culture surface, such as tissue culture dishes, plates, bottles, flasks, cell factory, etc. In certain embodiments, the solid support is a microcarrier, such as Cytodex 1 (GE Healthcare Life Sciences, Piscataway, N.J.); a macrocarrier, such as FibraCel (New Brunswick Scientific, Edison, N.J.), or a multilayered culture vessel, such as a CellCube (Corning Life Sciences, Lowell, Mass.) that permit medium perfusion.

In certain embodiments, the subject rHSV and/or rAAV vectors are produced as in vitro culture in eukaryote cells adapted to grow in suspension, such as a suspension culture of the BHK cell line.

In certain embodiments, the culture supernatant yields in excess of 1×10¹⁰ plaque-forming units (PFU) of rHSV, 1×10¹¹ plaque-forming units (PFU) of rHSV, 1×10¹² plaque-forming units (PFU) of rHSV, 1×10¹³ plaque-forming units (PFU) of rHSV, 1×10¹⁴ plaque-forming units (PFU) of rHSV.

In certain embodiments, vector stock is produced by one or more post processing steps, such as filtration and/or concentration (e.g., depth filtration, dead-end filtration, tangential flow filtration (TFF), and diafiltration), multi-column chromatography purification, final concentration/buffer exchange, etc., to obtain vector stocks with sufficient purity for administration to animals, including human. In certain embodiments, the purification process and the purified vector stock satisfy GMP standard.

In certain embodiments, the titer of the rHSV and/or rAAV vector stocks is about 1-2×10⁷ PFU/ml, about 1-2×10⁸ PFU/ml, about 1-2×10⁹ PFU/ml, about 1-2×10¹⁰ PFU/ml, about 1-2×10¹¹ PFU/ml, or about 1-2×10¹² PFU/ml.

In certain embodiments, the total yield of the rAAV vector stock is about 1-25×10¹⁴ total VG of purified rAAV, about 1-10×10¹⁴ total VG of purified rAAV, about 1-5×10¹⁴ total VG of purified rAAV, or about 2-4×10¹⁴ total VG of purified rAAV.

In certain embodiments, rHSV and/or rAAV vectors so produced are further purified from crude cell lysates by ion-exchange chromatography and/or by iodixanol density gradient centrifugation to ensure high final product purity. In certain embodiments, rAAV vectors so produced qualify as a clinical-grade vector batch.

In certain embodiments, the AAV production method of the invention further comprises determining the titer, purity, and/or potency of the rAAV vectors so produced.

This may include characterizing the purified rAAV stocks using one or more of: silver staining of SDS-PAGE separation of proteins to determine purity, qPCR to determine the ratio of rAAV full capsids to infectious particles (TCID₅₀), ELISA to determine the residual HSV protein, and qPCR to determine the residual HSV DNA.

7. Treatment of Muscular Dystrophy Using AAV Produced by the rHSV

The subject rHSV-based system can be used for large scale production of rAAV, which in turn can be used in gene therapy for treating various forms of muscular dystrophy, such as Duchenne's muscular dystrophy (DMD), Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), etc. In certain embodiments, the muscular dystrophy is DMD or BMD.

Thus another aspect of the invention provides a method of treating muscular dystrophy (such as DMD and BMD) in a subject in need thereof, the method comprising administering to the subject a therapeutically effective amount of a recombinant AAV (rAAV) vector encoding a functional version of the gene defective in the muscular dystrophy, such as a microdystrophin gene, wherein the rAAV is produced by the method of the invention using the subject rHSV vector and complementary system.

In certain embodiments, the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272 (all incorporated herein by reference).

In certain embodiments, the microdystrophin gene comprises a coding sequence for the R1, R16, R17, R23, and R24 spectrin-like repeats of the full-length dystrophin protein (such as one described in PCT/US2016/013733).

In certain embodiments, the method further comprises producing the rAAV by the method of the invention using the subject rHSV vector and complementary system, prior to administering to the subject the rAAV so produced.

### EXAMPLES

### Example 1 Detection of rHSV and rcHSV

Described herein are two assays that can be utilized to determine the presence of rHSV and rcHSV in HSV stocks, and ultimately in AAV stocks.

In one assay, HSV stocks are assayed on V27 cells, which produces ICP27, to determine the plaque-forming unit (PFU) titer of rHSV (which reproduction depends on ICP27 supplied in trans). In parallel, the same HSV stocks are also assayed on Vero cells, which do not produce ICP27, to assess the presence of any rcHSV (which reproduction does not depend on ICP27 supplied in trans).

This assay is based on the fact that, upon replication in the cell nucleus, rHSV or rcHSV induces a cytopathic effect (CPE), causing the infected cells to form plaques (Ye et al., 2014; Adamson-Small et al., 2016).

Detection of residual HSV is established to ensure the lowest detection limit possible. Currently, detection limits as low as 10-20 PFUs/mL have been described (Kang et al., 2009; Ye et al., 2011).

Alternatively, or additionally, a second, PCR-based assay for ICP27 is utilized for the detection of rHSV and/or rcHSV. One major limitation of this assay is that the assay will not indicate whether the viral particle is infectious. However, serial passaging may reveal whether the detected signal is amplified over time, which helps to determine whether the particles are replication competent and/or infectious.

### Example 2 New ICP27 Expression Cassettes Design

Described herein are several DNA sequences encoding ICP27 expression cassettes, useful for the generation of new adherent Vero, serum-free suspension adapted BHK cell lines, or any other cells permissive for herpes infection and thus supporting its propagation.

Such new ICP27-complementing cells, if used for propagating a new replication-defective rHSV-1 virus/vector with a complete deletion of ICP27-encoding UL54 gene, will have very low (if any) possibility of generating replication-competent rcHSV. The new ICP27-complementing cells can also be used to propagate the currently used d27-1 rHSV-based vectors, because of the smaller sequence overlap between the viral genome and the integrated ICP27 gene present in the cellular genome of the new adherent Vero or serum-free suspension adapted BHK cell lines.

Propagation of a new replication-defective rHSV-1 virus/vector with a complete deletion of the ICP27-encoding UL54 gene, in the subject new adherent Vero or serum-free suspension adapted BHK cell lines, will have no overlap between the ICP27 gene integrated in the cellular genome, and the viral genome of the rHSV with complete UL54 gene deletion. This enables a production of rHSV stock free or substantially free of any replication-competent rcHSV virus.

The DNA sequences encoding the smaller ICP27 expression cassettes (i.e., smaller than the ˜2.4 kb ICP27 expression cassette in the V27, 2-2 or B130 cells) are described in more detail below.

SEQ ID NO: 1 (2,188 nts) is a polynucleotide sequence that contains the 1951 nts HSV-1 (KOS 1.1) DNA sequence of UL54 gene (GenBank Accession KT887224; nts: 113,139-115,089), including 412 nts of UL54 promoter; 1,539 nts of ICP27 ORF sequence identical to HSV-1 KOS 1.1, encoding ICP27 HSV-1 strain KOS 1.1 ICP27 peptide (SEQ ID NO: 10; GenPept Accession AAF43147); and 237 nts of multiple restriction sites linker and bovine growth hormone polyadenylation (bGH poly(A)) signal sequence. Similar sequences may include replacing the bGH poly(A) signal sequence with any poly(A) signal sequence from other suitable sources, such as synthetic sequences or sequences from other eukaryotic genes or viruses.

SEQ ID NO: 2 (2,188 nts) is a polynucleotide sequence that contains 1,629 nts HSV-1 (KOS 1.1) DNA sequence of UL54 gene (GenBank Accession KT887224; nts: 113,139-114,767), including 412 nts of UL54 promoter; with first 1217 nts of ICP27 ORF sequence identical to HSV-1 KOS 1.1, and residual 322 nts of ICP27 codon optimized sequence encoding ICP27 HSV-1 strain KOS 1.1 ICP27 peptide (SEQ ID NO: 10; GenPept Accession AAF43147); and 237 nts of multiple restriction sites linker and bGH poly(A)) signal sequence. Similar sequences may include replacing the bGH poly(A) signal sequence with any poly(A) signal sequence from other suitable sources, such as synthetic sequences or sequences from other eukaryotic genes or viruses.

SEQ ID NO: 3 (2,188 nts) is a polynucleotide sequence that contains 412 nts HSV-1 (KOS 1.1) DNA sequence of UL54 gene (GenBank Accession KT887224; nts: 113,139-113,550), including 412 nts of UL54 promoter; complete 1,539 nts of codon optimized HSV-1 ICP27 ORF sequence encoding ICP27 HSV-1 strain KOS 1.1 ICP27 peptide (SEQ ID NO: 10; GenPept Accession AAF43147); and 237 nts of multiple restriction sites linker and bGH poly(A)) signal sequence. Similar sequences may include replacing the bGH poly(A) signal sequence with any poly(A) signal sequence from other suitable sources, such as synthetic sequences or sequences from other eukaryotic genes or viruses.

SEQ ID NO: 4 (2,447 nts) is a polynucleotide sequence that contains the same DNA sequence as it is in V27 cells (Rice and Knipe, 1990), compromised of 2,447 nts HSV-1 (KOS 1.1) DNA sequence of UL54 and UL55 genes (GenBank Accession KT887224; nts: 113,139-115,585), including 412 nts of UL54 promoter; 1,539 nts of ICP27 ORF sequence identical to HSV-1 KOS 1.1 encoding ICP27 HSV-1 strain KOS 1.1 ICP27 peptide (SEQ ID NO: 10; GenPept Accession AAF43147); and 496 nts of HSV-1 UL55 gene sequence.

SEQ ID NO: 5 (2,314 nts) is a polynucleotide sequence that contains 1,753 nts HSV-1 (KOS 1.1) DNA sequence of UL54 gene (GenBank Accession KT887224; nts: 113,013-114,765), including 538 nts of complete UL54 promoter; with first 1215 nts of ICP27 ORF sequence identical to HSV-1 KOS 1.1, and residual 324 nts of ICP27 codon optimized sequence encoding ICP27 HSV-1 strain KOS 1.1 ICP27 peptide (SEQ ID NO: 10; GenPept Accession AAF43147); and 237 nts of multiple restriction sites linker and bGH poly(A)) signal sequence. Similar sequences may include replacing the bGH poly(A) signal sequence with any poly(A) signal sequence from other suitable sources, such as synthetic sequences or sequences from other eukaryotic genes or viruses.

SEQ ID NO: 6 is a polynucleotide sequence that contains HSV-1 (KOS 1.1) DNA sequence of UL54 gene (GenBank Accession KT887224; nts: 113,139-113,550), including 412 nts of UL54 promoter; complete 1,539 nts of codon optimized HSV-1 ICP27 ORF sequence encoding ICP27 HSV-1 strain KOS 1.1 ICP27 peptide (SEQ ID NO: 10; GenPept Accession AAF43147); multiple restriction sites linker and bGH poly(A)) signal sequence. Similar sequences may include replacing the bGH poly(A) signal sequence with any poly(A) signal sequence from other suitable sources, such as synthetic sequences or sequences from other eukaryotic genes or viruses.

SEQ ID NO: 7 is a polynucleotide sequence that contains HSV-1 (KOS 1.1) DNA sequence of UL54 gene (GenBank Accession KT887224; nts: 113,139-113,550), including 412 nts of UL54 promoter; complete 1,539 nts of codon optimized HSV-1 ICP27 ORF sequence encoding ICP27 HSV-1 strain KOS 1.1 ICP27 peptide (SEQ ID NO: 10; GenPept Accession AAF43147); and 237 nts of multiple restriction sites linker and bGH poly(A)) signal sequence. Similar sequences may include replacing the bGH poly(A) signal sequence with any poly(A) signal sequence from other suitable sources, such as synthetic sequences or sequences from other eukaryotic genes or viruses.

SEQ ID NO: 8 (2,314 nts) is a polynucleotide sequence that contains 2,077 nts HSV-1 (KOS 1.1) DNA sequence of UL54 gene (GenBank Accession KT887224; nts: 113,013-115,089), including 538 nts of complete UL54 promoter; 1,539 nts of ICP27 ORF sequence identical to HSV-1 KOS 1.1 encoding ICP27 HSV-1 strain KOS 1.1 ICP27 peptide (SEQ ID: 10; GenPept Accession AAF43147); and 237 nts multiple restriction sites linker and bovine growth hormone polyadenylation (bGH poly(A)) signal sequence. Similar sequences may include replacing the bGH poly(A) signal sequence with any poly(A) signal sequence from other suitable sources, such as synthetic sequences or sequences from other eukaryotic genes or viruses.

SEQ ID NO: 9 (2,188 nts) is a polynucleotide sequence that contains 1,644 nts HSV-1 (KOS 1.1) DNA sequence of UL54 gene (GenBank Accession KT887224; nts: 113,139-114,782), including 412 nts of UL54 promoter; with first 1232 nts of ICP27 ORF sequence identical to HSV-1 KOS 1.1 and residual 307 nts of ICP27 codon optimized sequence encoding ICP27 HSV-1 strain KOS 1.1 ICP27 peptide (SEQ ID: 10; GenPept Accession AAF43147); and 237 nts multiple restriction sites linker and bGH poly(A)) signal sequence. Similar sequences may include replacing the bGH poly(A) signal sequence with any poly(A) signal sequence from other suitable sources, such as synthetic sequences or sequences from other eukaryotic genes or viruses.

SEQ ID NO: 10 is a polypeptide sequence of ICP27 HSV-1 strain KOS 1.1 ICP27 peptide (SEQ ID NO: 10; GenPept Accession AAF43147).

### Example 3 ICP27 Complementation Assays

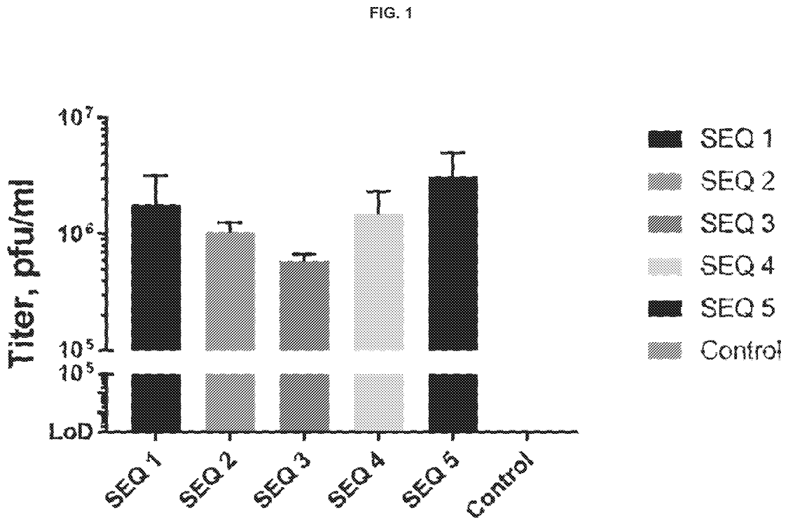

To test the ability of the new ICP27 expression constructs to support replication of rHSV, a series of complementation experiments were conducted. Specifically, BHK21 cells were infected with an rHSV at an MOI of 0.1, when transfected with plasmids bearing a polynucleotide sequence of either SEQ ID NO: 1, SEQ ID NO: 2, SEQ ID NO: 3, SEQ ID NO: 4, SEQ ID NO: 5, or an unrelated GFP plasmid control sequence. 24 hours post-transfection, cells were washed with PBS to remove transfection mix leftovers. Fresh medium was then added to each well. Cell supernatant samples were collected at 72 hours post infection and titrated on V27 cell monolayers using a standard plaque assay (see Example 1). The results from the complementation experiments are in FIG. 1.

The results demonstrated that all analyzed ICP27-expression constructs were able to support replication of rHSV with similar efficiency.

| | DNA | | HSV-1 | | | |
|---|---|---|---|---|---|---|
| | sequence | HSV-1 | ICP27 ORF | ICP27 | | |
| | identical to | ICP27 | identical to | Codon | | |
| | HSV-1 KOS | promoter | HSV-1 KOS | optimized | UL55 gene | |
| SEQ | 1.1 (nts of | (nts of | 1.1 | (length; | (nts of | |
| ID NO: | KT887224) | KT887224) | (length; nts) | nts) | KT887224) | PolyA |
| 1 | 113,139- | 113,139- | 1539 | 0 | No | bGH PolyA |
| | 115,089 | 113,550 | | | | |
| 2 | 113,139- | 113,139- | 1217 | 322 | No | bGH PolyA |
| | 114,767 | 113,550 | | | | |
| 3 | 113,139- | 113,139- | 0 | 1,539 | No | bGH PolyA |
| | 113,550 | 113,550 | | | | |
| 4 | 113,139- | 113,139- | 1539 | 0 | 115,090- | UL54 |
| | 115,582 | 113,550 | | | 115,582 | polyA |
| 5 | 113,013- | 113,013- | 1215 | 322 | No | bGH PolyA |
| | 114,767 | 113,550 | | | | |
| 6 | 113,139- | 113,139- | 0 | 1,539 | No | bGH PolyA |
| | 113,550 | 113,550 | | | | |
| 7 | 113,139- | 113,139- | 0 | 1,539 | No | bGH PolyA |
| | 113,550 | 113,550 | | | | |
| 8 | 113,013- | 113,013- | 1,539 | 0 | No | bGH PolyA |
| | 115,089 | 113,550 | | | | |
| 9 | 113,139- | 113,139- | 1232 | 307 | No | Yes |
| | 114,782 | 113,550 | | | | |

### Example 4 Generation of New ICP27-expressing Cell Lines: BHK-27 and Vero-27

BHK-27 and Vero-27 cell lines and related cell lines were generated either by transfecting BHK-21 or Vero cells, respectively, with the ICP27 sequence-bearing plasmid, or by infecting these cells with a 3^{rd} generation lentiviral vectors bearing ICP27 sequence defined by SEQ ID NO: 1, 2, 3, 5, 6, 7, 8, or 9. Stable clones were isolated under Geneticin selection, and tested for ICP27 expression by Western Blot and rHSV production by a standard plaque assay.

One of the BHK-27 clones, called BHK153, was used in Example 8 below.

### Example 5 Production of rHSV in BHK-27 and Vero-27

Isolated stable BHK-27 or Vero-27 cell clones were infected with the d27-1 rHSV at an MOI of 0.1 and incubated for 72 hours. rHSV titers in cell supernatants were determined by a standard plaque assay. Representative results demonstrate that the identified positive clones (such as clone #16, 50, 63, 110, and 153) which produced high levels of rHSV (FIG. 2).

### Example 6 Production of AAV Vectors from rHSV Produced in BHK-27 and Vero-27

rAAV vectors are produced by co-infecting either HEK 293 or BHK-21 cells with two rHSV vectors encoding either rAAV genome with GOI, or AAV Rep/Cap expression cassette, at an MOI of 2 for both rHSV. The rAAV vectors are harvested at 72 hours post-infection.

### Example 7 rHSV-1 Genome with a Complete Deletion of the UL54 Gene

The largest deletion of ICP27 gene reported in herpesvirus was a 1,624 bp deletion of ICP27 gene in d27-1 rHSV virus, which was generated by homologous recombination of pPsd27-1 plasmid constructed from pPs27pdl, after BamHI and StuI cleavage and circularization with ligation after BamHI site was blunted by Klenow DNA polymerase, generating 1,624 bp deletion in ICP27 gene (Rice and Knipe, 1990).

This virus is able to propagate in Vero-derived V27 cells which express ICP27. The V27 cells were generated by a stable transduction of the Vero cells with the pBH27 plasmid, which plasmid contains a 2.4-kb HSV-1 KOS 1.1 BamHI-HpaI DNA fragment with the ICP27 gene. There is a 815-bp large homologous sequence overlap between the d27-1 rHSV-1 virus/vector, and the ICP27 coding sequence has been integrated into the V27 cell genome (Rice and Knipe, 1988; Rice and Knipe, 1990).

Similarly, other Vero or BHK-21 based ICP27-expressing cell lines, 2-2 or B130, both carry a similar 2.4-kb BamHI-SstI ICP27 gene fragment from plasmid pSG130 B/S (Sekulovich et al., 1988; Smith et al., 1992; Howard et al., 1998).

SEQ ID NO: 11 (2,077 nts) is a polynucleotide sequence that represents 2,077 bp of HSV-1 (KOS 1.1) DNA sequence of the UL54 gene (GenBank Accession KT887224; nts: 113,013-115,089), which was deleted from the HSV-1 (KOS 1.1) genome to generate the subject new replication-deficient ICP27-deleted rHSV vector. Such new rHSV vector has so far the largest, and complete 2,077 bp deletion of the UL54 gene. It enables the production of rHSV stocks free of any replication-competent rcHSV virus, when it is used in conjunction with a new adherent Vero or serum-free suspension adapted BHK cell lines that have no overlapping sequence between the ICP27 gene integrated in the cellular genome, and the viral genome of the rHSV.

Deletion in other viruses from the Herpesvirales order will start from the first nucleotide after the termination codon of open reading frame (ORF) of the UL53 gene or its analogue, and up to the last nucleotide and including the termination codon of ORF of the UL54 gene or its analogue.

The present ICP27-deleted vector—SLD27—has 453 nts larger deletion in ICP27 gene (2,077 nts; SEQ ID NO: 11) than the d27-1 virus (1,624 nts; Rice and Knipe, 1990). Thus there will be no DNA sequence overlap between the SLD27 genome, and the ICP27 gene in complementing cell lines generated by using the plasmids of SEQ ID NOs: 1, 2, 3, 5, 6, 7, 8, and 9; except plasmid SEQ ID NO: 4.

Plasmid SEQ ID NO: 4 represents a HSV-1 DNA sequence in the V27 complementing cell line, with an overlap of 815 nts between the d27-1 virus and the V27 cell genome sequence.

Propagation of a new replication-defective rHSV-1 virus/vector with a complete deletion of the ICP27-encoding UL54 gene, in the new adherent Vero or serum-free suspension adapted BHK cell lines having no sequence overlap between the ICP27 gene integrated in the cellular genome and the rHSV viral genome with the complete UL54 gene deletion, enable production of rHSV stock free of replication-competent rcHSV virus.

The subject rHSV vectors and complementary cell lines expressing the ICP27 coding sequence deleted from the subject rHSV vectors can be used for large scale production of rAAV useful for gene therapy. See, for example, Thomas et al. (Scalable Recombinant Adeno-Associated Virus Production Using Recombinant Herpes Simplex Virus Type 1 Coinfection of Suspension-Adapted Mammalian Cells. Hum Gen Ther 20(8):861-870, 2009, entire content incorporated herein by reference); Adamson-Small et al. (A scalable method for the production of high-titer and high-quality adeno-associated type 9 vectors using the HSV platform. Hum Gene Ther Meth 28(1):1-14, 2017, entire content incorporated herein by reference); and Clement et al. (Large-Scale Adeno-Associated Viral Vector Production Using a Herpesvirus-Based System Enables Manufacturing for Clinical Studies. Human Gene Therapy 20:796-806, 2009, entire content incorporated herein by reference).

### Example 8 Production of rHSV Vector with Complete ICP27 Deletion

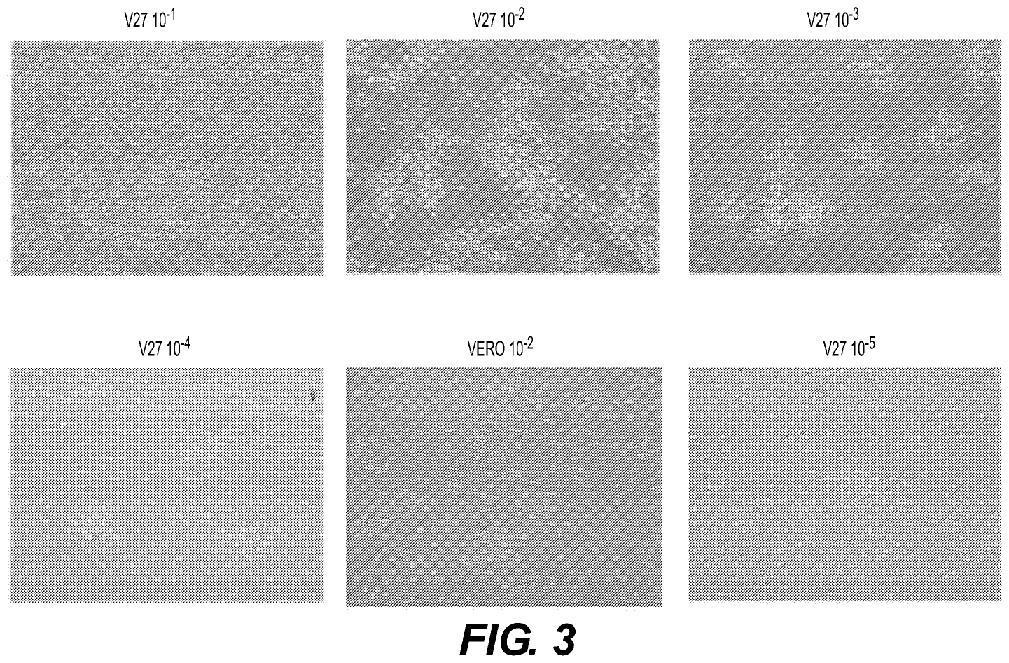

In this experiment, ICP27 gene (UL54), including its promoter and coding sequence, was completely deleted from the wild-type HSV-1 KOS 1.1 strain genome integrated in a bacterial artificial chromosome (BAC) vector (HSV-1 KOS 1.1-BAC), by using homologous recombination in electrocompetent E. coli. Four of the independently isolated ICP27-deleted HSV-1-BAC clones with 2.1-kb (2,077 bp) deletion in UL54 gene were tested using the V27 and Vero cell plaque assay described in Example 1, to show that robust production of ICP27-deleted HSV-1 virus can be achieved from these ICP27-deleted HSV-1-BAC clones with essentially no rcHSV contamination. Representative results from two of the 4 clones, Clone #3 and Clone #4, were shown in FIGS. 3 and 4 respectively.

Specifically, the ICP27 gene (UL54) was first completely deleted according to the present invention, by using homologous recombination in electrocompetent E. coli.

The homologous recombination in electrocompetent E. coli technology is based on homologous recombination and about 50 bp of homology regions on each side flanking a target DNA sequence. This is a system that can modify a target DNA, such as the wild-type HSV genome on a BAC vector, in particular the ICP27 gene, by deleting the target DNA.

One such electrocompetent E. coli strain is DY380, which is derived from the DH10B E. coli strain. Another such strain is SW102, which is derived from DY380. The galactose operon in SW102 has been modified, such that it is fully functional, except that the galactokinase gene (galK) has been deleted, but the galK function can be added in trans, thereby restoring the ability to grow on galactose as carbon source. This forms the biological basis for galK based selection in SW102.

The galK based selection is a two-step system involving both positive selection and negative selection. First, during the positive selection step, a galK cassette containing homology (e.g., at least 50 bp on each side) to a specified position in a BAC, such as the ICP27 gene locus, is inserted by homologous recombination into the BAC. The resulting recombinant bacteria are then able to grow on minimal media with galactose as the sole carbon source (positive selection). Second, the galK cassette is substituted by a donor sequence with homology flanking the galK cassette in the BAC vector. Successful recombinants can be identified by selecting against the galK cassette based on resistance to 2-deoxy-galactose (DOG) on minimal plates with glycerol as the carbon source. Although DOG itself is harmless, galK can phosphorylate DOG to become 2-deoxy-galactose-1-phosphate, a non-metabolizable and therefore toxic intermediate to the bacteria host. Thus only bacteria that have lost the galK cassette (e.g., by recombination) will survive and become DOG-resistant colonies (negative selection).

Using this system, a BAC carrying the wild-type HSV-1 KOS 1.1 strain was first introduced into the E. coli SW102 strain by electroporation. Next, a galK cassette with the galK coding sequence flanked on each side by about 50 bp of sequences homologous to genomic regions flanking the ICP27 gene was generated by PCR amplification. Here, the 50 bp homologous regions flanking the ICP27 gene were designed to eliminate the ICP20 gene completely, which is the 2,077 bp of HSV-1 KOS 1.1 DNA sequence of the UL54 gene (GenBank Accession KT887224; nts: 113,013-115,089). See Example 7 and SEQ ID NO: 11. This deletion includes the 538-bp UL54 promoter (nucleotides 1-538 of SEQ ID NO: 11), and the 1539-bp ICP27 coding sequence (nucleotides 539-2077 of SEQ ID NO: 11).

Following homologous recombination, galK positive selection was performed to identify recombinants that had replaced the 2,077-bp ICP27 gene with the galK cassette.

Next, galK was similarly eliminated from the BAC vector using homologous recombination by using ICP27 flanking sequences (linking about 50 bp upstream of nucleotide 113,013 in KT887224, to about 50 bp downstream of nucleotide 115,089 in KT887224). Following this step, galK negative selection was performed to identify recombinants that had lost the galK cassette. The resulting BAC clones were named ICP27-deleted HSV-1-BAC clones. Four of such clones, namely Clones 1-4, were subject to further testing to show their ability to support robust rHSV production with essentially no contaminating rcHSV.

Specifically, BHK153 cells (see Example 4) were transfected by BAC DNA from individual ICP27-deleted HSV-1-BAC clones (i.e., Clones 1-4). The BAC DNA sequence from ICP27-deleted HSV-1-BAC clones share no homologous region with the ICP27 gene stably integrated in the BHK153 cells, which have previously been confirmed to be able to provide ICP27 function for rHSV production (see FIG. 2). Lysates of the transfected BHK153 cells were collected about 12-13 days post transfection, and supernatants containing rHSV were assayed using the method of Example 1 in 6-well plates, to determine rHSV viral titer on V27 cells and Vero cells. The results 2 days post infection for Clones 3 and 4 were shown in FIGS. 3 and 4, respectively.

It is apparent that the rHSV vector of the invention with 2,077-bp deletion of the ICP27 gene is fully capable of supporting robust rHSV production. In V27 cells that express ICP27, all four ICP27-deleted HSV-1-BAC clones generated plaques on V27 cells, in all serial dilutions (10⁻¹, 10⁻², . . . , and 10⁻⁶). See FIGS. 3 and 4. There were ˜2-10 plaques on the 10⁻⁶ dilutions, and the cytopathic effect (CPE) was observed on 10⁻¹ and 10⁻² dilutions, which represent 100 μL and 10 μL of the harvest, respectively.

No plaques were observed from any of the four ICP27-deleted HSV-1-BAC clones on Vero cells (which lack the ICP27 function required for ICP27-deleted HSV-1 virus propagation), in serial dilutions 10⁻¹ and 10⁻². See FIGS. 3 and 4.

As a control, CPE was observed on Vero cells in serial dilutions 10¹, 10⁻², and 10⁻³, after infection with wild-type HSV-1 KOS 1.1-BAC virus generated previously by transfection of HSV-1 KOS 1.1-BAC DNA on BHK153 cells (data not shown).

### Example 9 Generation of rHSV Vectors with Complete ICP27 Deletion and Replacement of the TK Locus with Either a Human Dystrophin Minigene or an AAV Rep/Cap Coding Sequence

In this example, the ICP27-deleted HSV-1-BAC vector in Example 8 further modified by homologous recombination to replace the HSV-1 thymidine kinase (TK) gene locus (UL23), with either a gene of interest (GOI), such as a human dystrophin minigene (microdystrophin), or any AAV rep/cap expression cassette required for rAAV production. Co-infection of a suitable producer cell line with the two rHSV vectors (rHSV GOI; and rHSVrep/cap) can be used to generate rAAV gene therapy vectors for gene therapy.

One way to accomplish this is by using a two-step process using galK selection as described above. First, the TK locus (UL23) is replaced with a galK cassette via electroporation and recombination. The galK cassette is amplified by PCR using two primers, each primer flanks the galK cassette and amplifies sequences of at least a 50-bp sequence on each side of the insertion site within the HSV-1 TK locus sequence (UL23). The resulting PCR product has the galK coding sequence, flanked by two 50-bp TK homologous regions for electroporation and recombination. The resulting PCR product is then introduced into E. coli SW102 having ICP27-deleted HSV-1-BAC clones (see Example 8) to perform homologous recombination. Positive galK selection resulted in a modified ICP27-deleted HSV-1-BAC genome, having both complete ICP27 deletion and a galK gene insertion into HSV TK gene (ICP27-deleted HSV-1-BAC TK^{mut}/galK⁺).

In the next step, galK is replaced with either a GOI or rep/cap DNA cassettes via electroporation and recombination, resulting in a pair of rHSV vectors (rHSV GOI and rHSVrep/cap) for rAAV production.

Alternatively, as a different approach, the galK selection marker was first inserted into either the AAV rep/cap expression cassette or the GOI, to generate the galK-labeled rep/cap expression cassette or galK-labeled GOI, before the galK-labeled rep/cap expression cassette or the galK-labeled GOI was inserted into the ICP27-deleted HSV TK locus using homologous recombination. Data not shown.

Further alternatively, one could construct a single rHSV rep/cap vector for production of AAV using a producer cell line (PCL) with a stably integrated GOI cassette flanked by the AAV ITR sequences.

One of such pair of rHSV vectors has a gene of interest (GOI) flanked by the AAV ITR sequences. Specifically, a gene of interest may be a dystrophin minigene described in PCT/US2016/013733 (published as WO/2016/115543, incorporated herein by reference). One such specific microdystrophin gene comprises a coding sequence for the R1, R16, R17, R23, and R24 spectrin-like repeats of the full-length dystrophin protein, under the transcriptional control of the CK8 promoter, and is referred to herein as “CK8-HuDys5”. The gene of interest flanked by the AAV ITR sequences is further flanked by 50-bp homologous regions required for electroporation and recombination (for example, TK-ITR-CK8-HuDys5-ITR-TK). The entire construct can be carried on a plasmid (e.g., pJ234TK-ITR-CK8-HuDys5-ITR-TK-Final), which can be linearized by NruI and ZraI before homologous recombination in ICP27-deleted HSV-1-BAC TK^{mut}/galK+ in an E. coli (SW102 as an example). After galK negative selection, the clones are screened for successful recombinants with galK removed and having the GOI cassette inserted in the TK location (ICP27-deleted HSV-1-BAC TK^{mut}/ITR-GOI-ITR). This is a modified rHSV BAC vector, comprising a completely deleted ICP27 (UL54) gene, a TK^{mut} gene, the latter of which is replaced by a human dystrophin minigene under the control of the CK8 promoter flanked by AAV ITR sequences.

Further, one could construct a single rHSV rep/cap vector for production of AAV using a producer cell line (PCL) with a stably integrated GOI cassette flanked by the AAV ITR sequences.

Using substantially the same approach, any AAV rep/cap expression cassette can be inserted into the TK (UL23) locus of ICP27-deleted HSV-1-BAC TK^{mut}/galK+, to result in ICP27-deleted HSV-1-BAC TK^{mut}/rep/cap, after performing electroporation and recombination on ICP27-deleted HSV-1-BAC TK^{mut}/galK+ with galK negative selection. The rep/cap cassette can be generated by PCR amplification using primers with 50-bp sequence homologous regions flanking the TK locus.

In the above constructs, the HSV genome was inserted in a pBAC plasmid, and that inserted sequence was flanked by loxP sequences. The BAC sequence can be removed from the ICP27-deleted HSV-1-BAC TK^{mut}/ITR-GOI-ITR, and the ICP27-deleted HSV-1-BAC TK^{mut}/rep/cap backbones, by co-transfecting the respective BAC vectors and a Cre-expressing plasmid into ICP27 expressing BHK or Vero cells. Plaque purification of the progeny viruses lacking BAC sequence results in virus ICP27-deleted HSV-1 TK^{mut}/ITR-GOI-ITR, and ICP27-deleted HSV-1 TK′/rep-cap.

For example, a rAAV vectors can be produced using these two rHSV vectors, according to Example 6. The resulting rAAV vectors have within the ITR sequences a human dystrophin minigene HuDys5 under the control of the muscle specific CK8 promoter, and can be readily used in gene therapy to treat muscular dystrophy.

A slight variation of the process above may include directly replacing any GOI by any other GOI using homologous recombination. For example, after the generation of the ICP27-deleted HSV-1 TK^{mut}/ITR-GOI-ITR clone, the GOI may be directly replaced by another GOI, via galK selection. For example, if the promoter and the polyA regions of the old and new GOI's are the same, they can serve as the 50-bp minimal homologous regions for homologous recombination. If these sequences are different, then the spacer regions can be used (if they are at least 50 bp in length).

Specifically, assuming the promoter and polyA regions are identical, the galK cassette can first be inserted into ICP27-deleted HSV-1 TK^{mut}/ITR-GOI-ITR between the promoter and polyA sequences of the GOI coding sequence by electroporation and recombination. Following galK positive selection, ICP27-deleted HSV-1 TK^{mut}/ITR-promoter-galK-polyA-ITR is obtained in which the coding sequence of GOI, between its promoter and the polyA signal sequence, is replaced by galK. Next, the inserted galK cassette in ICP27-deleted HSV-1 TK^{mut}/ITR-prom-galK-polyA-ITR is replaced by the coding sequence from another GOI, via electroporation and recombination. ICP27-deleted HSV-1 TK^{mut}/ITR-GOI-ITR is obtained following galK negative selection.

Similarly, an AAV rep-capX expression cassette can be inserted into the deleted TK (UL23) locus of ICP27-deleted HSV-1 TK^{mut}/galK+, to result in ICP27-deleted HSV-1 TK^{mut}/rep-capX, after performing electroporation and recombination on ICP27-deleted HSV-1 TK^{mut}/galK+ with galK negative selection.

The BAC sequence can be removed from the ICP27-deleted HSV-1-BAC TK^{mut}/ITR-GOI-ITR, and the ICP27-deleted HSV-1-BAC TK′/rep/capX backbones, by co-transfecting the respective BAC vectors and a Cre-expressing plasmid into ICP27 expressing BHK or Vero cells. Plaque purification of the progeny viruses lacking BAC sequence results in virus ICP27-deleted HSV-1 TK^{mut}/ITR-GOI-ITR, and ICP27-deleted HSV-1 TK^{mut}/rep-capX.

### Example 10 Generation of rHSV Vectors with Complete ICP27 Deletion and Replacement of the TK Locus with Either a Human Dystrophin Minigene or a AAV Rep/Cap Coding Sequence—FRT Constructs

In this example, and similar to Example 9, the ICP27-deleted HSV-1-BAC vector in Example 8 is further modified by inserting into the HSV-1 TK gene locus (UL23) either a gene of interest, such as a human dystrophin minigene, or an AAV rep/cap expression cassette required for rAAV production. The difference between this example and Example 9 is that the galK cassette in the intermediate construct, as well as the GOI (for example the dystrophin minigene) and the rep-cap expression cassette, are all flanked by frtG and frtH sequences to facilitate easier exchange of constructs. Thus electroporation and recombination in E. coli is only used initially to replace the TK locus with the galK cassette flanked by frtG and frtH sites, and galK can then be replaced by either the GOI or the rep-cap expression cassette via FLP recombination.

This is again accomplished in a two-step process using galK selection as described above. First, the TK locus (UL23) is replaced with a frtG-galK-frtH cassette via electroporation and recombination. The frtG-galK-frtH cassette is amplified by PCR with two primers, each having a 50-bp sequence homologous to a sequence flanking the HSV-1 TK locus (UL23). The resulting PCR product has the galK coding sequence in the middle, flanked by frtG and frtH, which are in turn flanked by two 50-bp TK homologous regions for homologous recombination. The PCR product is then introduced into E. coli (for example, SW102) having the ICP27-deleted HSV-1-BAC clones (see Example 8) for homologous recombination. Positive galK selection results in a modified rHSV on BAC vector, having both complete ICP27 deletion and TK deletion, and having galK flanked by frtG and frtH sites (ICP27-deleted HSV-1-BAC TK^{mut}/frtG-galK-frtH). This BAC clone is then used subsequently as acceptor for any ITR-GOI-ITR cassettes, and any rep-cap cassettes, via FLP recombination.

In the next step, galK is replaced with either one of two constructs via FLP recombination, resulting in a pair of rHSV vectors useful for rAAV production.

One of such rHSV vectors has a gene of interest (GOI) flanked by the AAV ITR sequences, further flanked by frtG and frtH sites. Specifically, a gene of interest may be a dystrophin minigene described in PCT/US2016/013733 (published as WO/2016/115543, incorporated herein by reference). One such specific microdystrophin gene comprises a coding sequence for the R1, R16, R17, R23, and R24 spectrin-like repeats of the full-length dystrophin protein, under the transcriptional control of the CK8 promoter, and is referred to herein as “CK8-HuDys5.” Alternatively, any of a different dystrophin minigene, such as those described herein above, may be inserted.

The gene of interest flanked by the AAV ITR sequences is further flanked by the frtG and frtH sites (frtG-ITR-GOI-ITR-frtH). The entire construct can be carried on a transfer plasmid for FLP recombination on ICP27-deleted HSV-1-BAC TK^{mut}/frtG-galK-frtH. After galK negative selection, successful recombinants having lost galK can be obtained as ICP27-deleted HSV-1-BAC TK^{mut}/frtG-ITR-GOI-ITR-frtH, a modified rHSV vector on the BAC vector, comprising a completely deleted ICP27 (UL54) gene and TK gene (UL23), the latter of which is replaced by a GOI flanked by AAV ITR sequences and frtG-frtH sites.

Using substantially the same approach, an AAV rep-capX (for any suitable/desired AAV capsids) expression cassette can be inserted into the TK locus (UL23) locus of ICP27-deleted HSV-1-BAC TK^{mut}/frtG-galK-frtH, resulting in ICP27-deleted HSV-1-BAC TK^{mut}/frtG-rep-capX-frtH, after performing FLP recombination followed by galK negative selection.

The loxP-flanked BAC backbone in ICP27-deleted HSV-1-BAC TK^{mut}/frtG-ITR-GOI-ITR-frtH, and the loxP-flanked BAC backbone in ICP27-deleted HSV-1-BAC TK^{mut}/frtG-rep-capX-frtH can both be eliminated by co-transfecting the respective BAC vectors and a Cre-expressing plasmid into Vero cells. Plaque purification of the progeny viruses lacking beta galactosidase results in virus ICP27-deleted HSV-1-BAC TK^{mut}/frtG-ITR-GOI-ITR-frtH, and ICP27-deleted HSV-1-BAC TK^{mut}/frtG-rep-capX-frtH.

rAAV vectors can be produced using these two rHSV vectors, according to Example 6. The resulting rAAV vectors have within the ITR sequences any GOI, such as a human dystrophin minigene, and can be readily used in gene therapy to treat muscular dystrophy.

### Example 11 Generation of rHSV Vectors with Complete ICP27 Deletion and Insertion of AAV Rep/Cap Coding Sequence in the TK (UL23) Locus by Homologous Recombination in ICP27-Complementing Cells Followed by Acyclovir Selection of HSV Clones

In this example, and similar to Example 9, the ICP27-deleted HSV-1-BAC vector in Example 8 is further modified by inserting into its HSV-1 TK gene locus (UL23) expression cassette with an AAV rep/cap expression cassette required for rAAV production. The difference between this example and Example 9 is that the homologous recombination is performed after co-transfection in eukaryotic ICP27-complementing cells instead of electroporation in the suitable E. coli strain, and the selection marker used for selection of HSV clones is acyclovir (ACV) against the clones with intact TK gene, instead of selection for and/or against galK cassette in the intermediate constructs.

Similar if not identical approaches can also be used to insert any gene of interest, such as a human dystrophin minigene, or others, into the TK locus.

Specifically, acyclovir is an antiviral medication first developed in 1974, primarily used for the treatment of herpes simplex virus infections, chickenpox, shingles and other herpes viruses. It is also available as a laboratory reagent. Acyclovir is a nucleoside analog that is converted by the herpes TK (thymidine kinase) enzyme to acyclovir monophosphate, which is then converted by host cell kinases to acyclovir triphosphate (ACV-TP). ACV-TP is a competitive inhibitor that inactivates herpes-specified DNA polymerases, hence preventing further viral DNA synthesis without affecting the normal cellular processes.

Using homologous recombination, the GOI (such as the human dystrophin minigene) or in this case, the AAV rep/cap8 expression cassette can be flanked by homologous regions surrounding the HSV TK gene locus UL23 and be used as donor DNA to inactivate the TK gene in the HSV vector. Only HSV having lost the TK gene function due to homologous recombination (and hence simultaneously acquiring the GOI or the rep/cap cassette) can survive growing in the presence of acyclovir.

Using this approach, a repcap expression cassette flanked by about 50 bp of homologous regions surrounding the HSV TK locus on each side of the expression cassette was used to inactivate the TK locus (UL23) on the ICP27-deleted HSV-1-BAC vector, based on acyclovir selection at 22.5 μg/mL. Specifically, V75.4 cells, a Vero-derived cells line, expressing a functional ICP-27 gene that has no overlap sequence with the ICP27-deleted HSV-1-BAC vector, were co-transfected with the ICP27-deleted HSV-1-BAC vector (pre-treated with Cre), and a plasmid containing the donor DNA encompassing the repcap8 expression cassette flanked on both sides by about 50 bp of homologous regions surrounding the HSV TK locus. The V75.4 cells were then cultured in the presence of about 22.5 μg/mL of acyclovir to select for clones that presumably have inactivated the HSV TK locus via homologous recombination. Acyclovir-resistant clones were observed at 3 to 7 days post infection (3-7 dpi), and the presence of repcap8 expression cassette was confirmed by a qPCR assay.

This same selection scheme can also be used when inserted into the TK locus of the ICP27-deleted HSV-1-BAC vector of the invention with a GOI flanked by AAV ITR sequences, such as a minidystrophin expression cassette flanked by AAV ITR sequences.

rAAV vectors can then be produced using these two rHSV vectors, according to Example 6. The resulting rAAV vectors have within the ITR sequences any GOI, such as a human dystrophin minigene, and can be readily used in gene therapy to treat muscular dystrophy.

### Example 12 Generation of V75.4 Cell Line

This example shows the establishment of a packaging cell line (V75.4 cell line) for producing HSV vectors having deleted ICP27 gene. The packaging cell line contains an ICP27-encoding sequence that is designed to have no sequence overlap with the HSV vector of the invention with a complete deletion of the ICP27 gene.

The parental cell line used for the generation of V75.4 Cell Line was a Vero cell line CCL-81, obtained from American Type Culture Collection (ATCC) (Manassas, Va.). Vero cell line is an African green monkey (Cercopithecus aethiops) kidney cell line which is highly susceptible to various types of viruses, including herpes simplex virus 1 (HSV-1). Vero cells were stably transduced by a lentivirus vector generated from a lenti-provirus plasmid, by inserting the nucleotide sequence UL54-002 (promoter and codon-optimized ORF) SEQ ID NO: 12. The ICP27 expression cassette stably integrated in the V75.4 cells contains woodchuck hepatitis virus post-transcriptional regulatory element (WPRE) sequence and the lentivirus polyadenylation signal from 3′ LTR lentivirus sequence, as is shown in SEQ ID NO: 13.

### Transduction, Cloning and Cell Line Identification

Vero cells maintained in DMEM plus 5% FBS at 37° C. in a humidified CO₂-controlled incubator with 5% CO₂ were transduced with lentivirus vector PR-UL54-002, at multiplicity of infection (MOI)=10. Cells from this pool were cloned by plating in different densities into 15 cm² plates, and were maintained in DMEM plus 10% FBS without selection at 37° C. in a humidified CO₂-controlled incubator with 5% CO₂. After 2-3 weeks, the single colonies-master wells (MWs) were detached by trypsin and harvested using cloning cylinders. Cells from master wells (MWs) were seeded into 24-well plates and maintained in the same medium and conditions. As the wells reached confluence, clones were detached using trypsin and expanded to two 24-well plates (one for terminal replica for screening and one for expansion). The portion of the culture slated for HSV production screening was infected with Δ27HSV at MOI=0.15 and harvested after 72 hrs and tested for DDPCR titer for HSV UL36 copies and plaque per mL.

Four outperforming MWs were identified and shown in Table 1.

TABLE 1
Outperforming MWs DDPCR (UL36) and
plaque titers per mL

| | ddPCR | V27 |
|---|---|---|
| | TITER | TITER |
| MW CLONE | (VG/mL) | (PFU/mL) |
| LV-Vero002 #28 | 3.61E+08 | 2.00E+06 |
| LV-Vero002 #36 | 4.11E+08 | 1.20E+07 |
| LV-Vero002 #75 | 5.49E+08 | 2.20E+07 |
| LV-Vero002 #76 | 4.07E+08 | 8.00E+06 |

Ultimately the highest level of production was found in MW LV-Vero002 #75 (MW75) that had been selected for further subcloning.

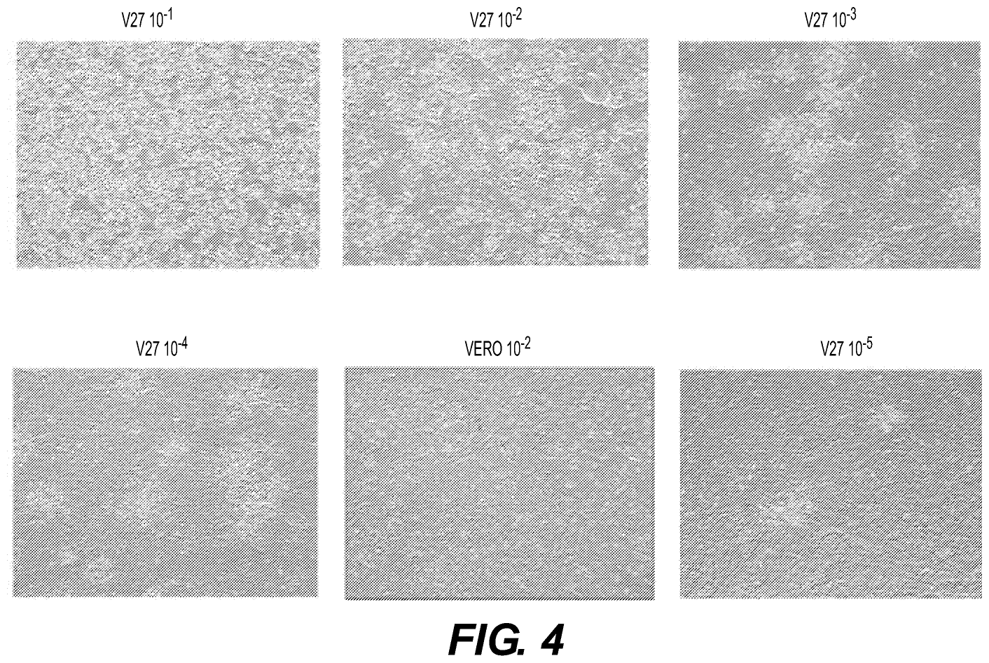

Cells from the MW75 were cloned by limiting dilution seeded at a density of 0.3 cells/well in 96-well plates in the same medium and conditions as described previously. The plates were visually inspected to identify those wells seeded with only a single cell. After a week of growth, the media was replaced and in the wells that reached confluence, clones were detached using trypsin and expanded to 24 well plates. As the wells reached confluence, clones were detached using trypsin and expanded to two 24-well plates (one for terminal replica for screening and one for expansion). The portion of the culture slated for HSV production screening was infected with Δ27HSV at MOI=0.15 and harvested after 72 hrs and tested for DDPCR titer for HSV UL36 copies. The outperforming MW75 subclones were selected by UL36 DDPCR as shown in FIG. 5.

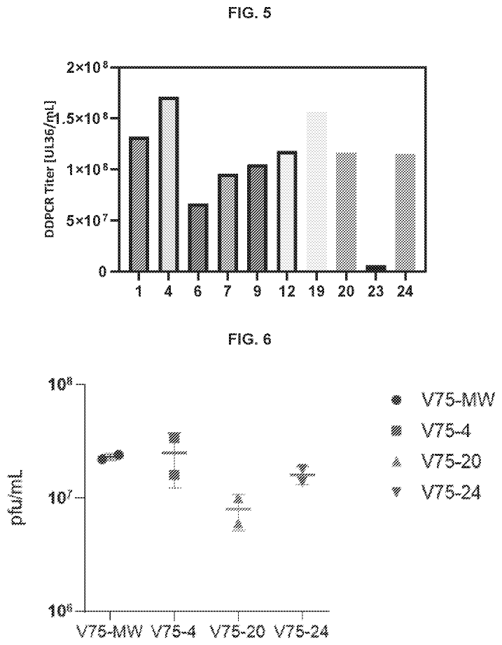

The subclones were narrowed down to clones #4, #20 and #24, and were assessed for the viability, coupling time, and generational stability up to passage P26. The stability and the yield of Δ27HSV virus was the best in MW75 clone #4 (hence the V75.4 cell line) (FIG. 6), which was selected for further use in the rc-HSV-free system of the invention.

The ICP-27 coding sequence in these newly established packaging cell lines, including V75.4, contains a codon optimized region encoding the C-terminal end of the ICP27 protein. The codon optimization was designed to have least sequence homology, at the nucleic acid level, compared to the remnant ICP27 coding sequence in the d27-1 HSV vector currently wildly used. This was designed to minimize the chance of generating rcHSV viral particles, when the traditional d27-1 HSV vector is packaged in the subject V75.4-type packaging cell lines, since any sequence overlap between the remnant ICP27 coding sequence on the d27-1 HSV vector and the ICP27 encoding sequence in the subject packaging cell line will be reduced to 67% or less due to codon degeneracy.

Indeed, preliminary results (data not shown) indicated that no rcHSV viral particles were generated when the traditional d27-1 HSV vector was packaged in V75.4 cell line, to the extent that there was no contaminating rcHSV in the d27-1 HSV stock.

### Example 13 Lack of Detectable rcHSV in HSV Preparation Prepared in the V75.4 Cell Line

This example demonstrates that the subject HSV vector and packaging cell line, when used together, produce HSV stock with no detectable rcHSV revertants.

Specifically, several subject HSV vectors with a complete ICP-27 deletion (“SLB27” vectors herein), and either a rep/cap expression cassette (SLB27-RC9 #1, #2 and #3) or a GOI flanked by ITR sequences (SLB27-goi #1 and #2) inserted into the TK locus of the HSV vector were tested. These vectors were used to infect the subject V75.4 packaging cell line to propagate and produce the first passage (P1) of these vectors. The P1 vectors were then used to infect V75.4 cells to produce the second passage (P2) of these vectors. The P2 passage HSV vectors were then tested either in Vero cells (which has no functional ICP27) or V75.4 cells (which has functional ICP27), and ddPCR was then used to detect any rcHSV that may have resulted after undesirable recombination events between the subject ICP27-deleted HSV vectors and the HSV ICP27 fragment in the host cell (V75.4).

ddPCR was used here due to its ability to provide an absolute count of target DNA copies per input sample without the need for running standard curves, with unparalleled precision and increased signal-to-noise ratio.

The data showed that no rcHSV was detected (BLQ, or “Below Limit of Quantification”) in P2 of any of the SLB27 preparations propagated in the subject V75.4 cells, or when P2 was amplified in Vero cells. Indeed, no rcHSV was detected by Vero plaque assays in any of the SLB27 preparations propagated in the subject V75.4 cells up to passage P8.

As a control, traditional HSV vectors with a partial ICP27 deletion and a similar GOI insertion in the TK locus (Δ27HSVgoi) has significant amount of rcHSV revertants, especially when the supposedly rcHSV-free HSV viral stock was tested in Vero cells (see table below).

ddPCR Results for ICP27 Titer/mL of Media

| | ICP 27 Titer | ICP 27 Titer | ICP 27 Titer |
|---|---|---|---|
| | in original | From P2 in | In Vero cells |
| HSV Type | samples | V75.4 cells | medium |
| SLB27-RC9 #1 | BLQ | BLQ | BLQ |
| SLB27-RC9 #2 | BLQ | BLQ | BLQ |
| SLB27-RC9 #3 | BLQ | BLQ | BLQ |
| SLB27-goi #1 | BLQ | BLQ | BLQ |
| SLB27-goi #2 | BLQ | BLQ | BLQ |
| Δ27HSVgoi | 5.55E+05 | 3.65E+05 | 8.40E+07 |

Limit of Quantification of ddPCR ICP27 Titer Assay

| | | rcHSV |
|---|---|---|
| Sample | ICP27 Titer | PFU/mL |
| rcHSV dil.10⁻¹ | 8.10E+08 | 4.50E+07 |
| rcHSV dil.10⁻² | 6.66E+07 | 4.50E+06 |
| rcHSV dil.10⁻³ | 6.67E+06 | 4.50E+05 |
| rcHSV dil.10⁻⁴ | 4.60E+05 | 4.50E+04 |
| rcHSV dil.10⁻⁵ | 7.70E+04 | 4.50E+03 |
| rcHSV dil.10⁻⁶ | BLQ | 4.50E+02 |
| V75 medium + DNAse | BLQ | — |
| V75 medium only | 1.72E+05 | — |

### Example 14 Higher Titer of HSV Vectors with Complete vs. Incomplete ICP27 Deletion when Propagated in V75.4 Cell Line

This example shows that the subject HSV vectors, when propagated in the subject packaging cell line such as V75.4, produced HSV stocks with higher titer than traditional HSV vectors having incomplete ICP27 deletion.

This result is surprising since the subject HSV vectors have a larger genomic deletion at the ICP27 locus, compared to the traditional HSV vector having only a partial deletion of the same locus. Thus it would be expected that the subject HSV vectors are less “healthy” compared to the traditional HSV vectors. Indeed, prior study showed that a larger deletion in HSV than the commonly used d27-1 HSV strain (the d27-1 strain has a partial ICP27 deletion that left behind the C-terminal region coding sequence of ICP27 in the resulting HSV vector) did not grow well in the V27 packaging cell line, and the infected V27 cells took markedly longer to develop. More importantly, the viral titer of the harvested HSV was 5- to 10-fold lower (Bunnell, Ph.D. Thesis, Univ. of Alberta, 2001).

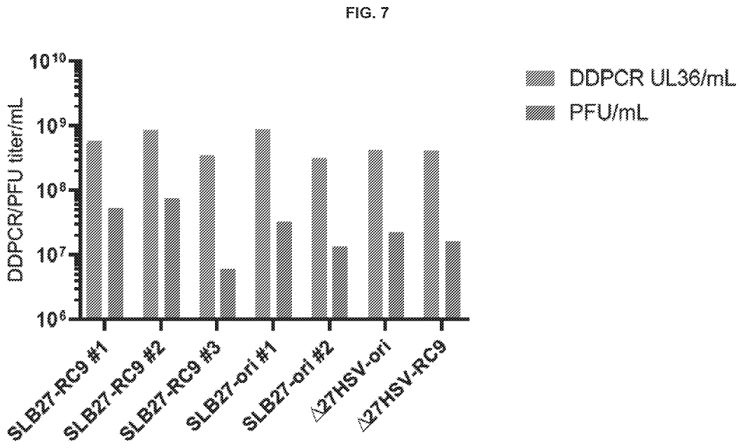

Thus it came as a surprise that the titer of the subject HSV vector having a larger (complete) deletion than the traditional d27-1 HSV strain (with incomplete ICP27 deletion) actually produced a higher titer compared to d27-1, when both were propagated in the subject V75.4 packaging cell line. As shown in FIG. 7, up to 2-fold (100%) higher titers of the subject SLB27 vectors (see Example 13) were observed compared to the Δ27HSV vectors in the V75.4 cells. Note the log scale of the titer axis.

It is also surprising that a high to very high percentage of syncytial plaque phenotype was observed in infected packaging cells (i.e., V75.4 cells in this case) when the subject SLB27 HSV vectors with complete ICP27 deletion was used, as compared to the same cells infected by d27-1 HSV vectors. Syncytial plaque was formed by fusion of an infected cell with its neighboring (uninfected) cells, leading to the formation of multi-nucleate enlarged cells, which probably led to a more efficient HSV production. Syncytial plaque formation can be shown as higher genome copy (as measured by ddPCR) and infectious (plaques) titers/mL.

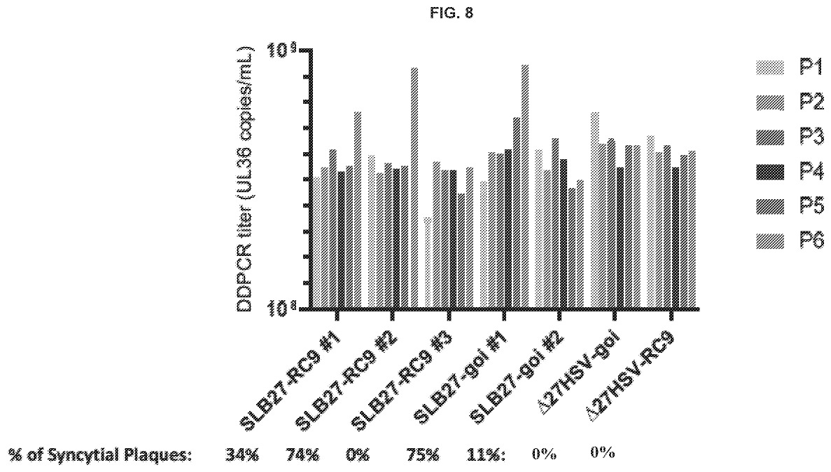

Indeed, as shown in FIG. 8, almost all cultures of V75.4 cells infected by the subject SLB27 HSV viruses developed a syncytial plaque phenotype in passage 6 (P6). One of the clones (SLB27-RC9 #2) had 74% of syncytial plaques in the infected V75.4 cells, another clone (SLB27-goi #1) had 75% of syncytial plaques in the infected V75.4 cells. In contrast, none of the corresponding V75.4 cultures infected by d27-1 HSV vectors exhibited syncytial plaque formation under the same conditions.

These surprising findings demonstrate that complete deletion of ICP27 in the HSV vector, coupled with a complementary ICP27 coding sequence in the packaging cell, not only essentially eliminated detrimental rcHSV generation in the HSV viral stocks, but also unexpectedly led to more efficient HSV viral production, potentially through high percentage of syncitial plaque formation.

### Example 15 Higher Titer of AAV Vectors Prepared Using the HSV Vectors with Complete Vs. Incomplete ICP27 Deletion when Propagated in BHK Cell Line

This example shows that the subject HSV vectors having either AAV rep/cap coding sequence or GOI flanked by AAV ITR sequences, when used to co-infect a suitable AAV production cell line such as BHK, can produce higher AAV titer than that produced by similar HSV vectors with incomplete ICP27 deletion.

Specifically, two HSV vectors of the invention with complete ICP27 deletion, one with AAV9 rep/cap coding sequence in the TK locus, another with a dystrophin minigene flanked by AAV ITR sequences, were propagated in the subject V75.4 packaging cell line to harvest HSV stocks. The harvested HSV stocks were then used to infect the AAV production cell line BHK to produce AAV particles. The resulting AAV titer was determined, and compared to that similarly produced AAV using the traditional HSV vectors having incomplete ICP27 deletion (d27HSV).

The same amount of virus or multiplicity of infection (MOI=2), and the same number of cells were used in each experiments (1×10⁶ cells per experiment; n=3 for each group).

In both Δ27HSV-goi #3 and Δ27HSV-RC9 vectors used for the AAV yield experiment, rcHSV and ICP27 were detected, consistent with previous findings. In contrast, no rcHSV contamination and ICP27 were detected in the corresponding SLB27-RC9 #2 and SLB27-goi #2 HSV vector stocks.

ddPCR ICP27 Assay for HSV Stocks Used
in the AAV Production Experiment

| Sample | ICP27 Titer |
|---|---|
| SLB27-RC9 #1 | BLQ |
| SLB27-RC9 #2 | BLQ |
| SLB27-RC9 #3 | BLQ |
| SLB27-goi #1 | BLQ |
| SLB27-goi #2 | BLQ |
| Δ27HSVgoi #1 | BLQ |
| Δ27HSVgoi #2 | 1.24E+06 |
| Δ27HSVgoi #3 | 5.55E+05 |
| Δ27HSV-RC9 | 1.83E+06 |

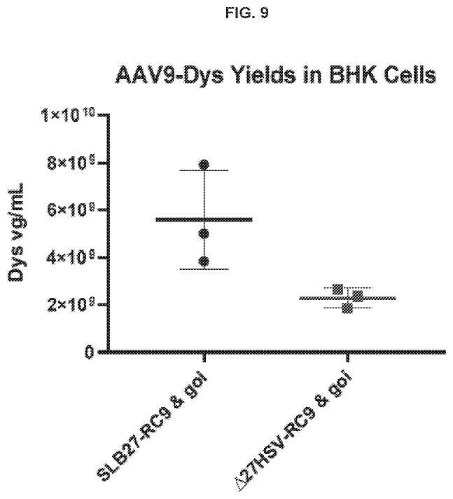

As shown in FIG. 9, the AAV titer was on average about 5.5×10⁹, compared to that of about 2.5×10⁹. It is possible that the much higher AAV titers associated with using the subject HSV vector and packaging cell line were due to the lack of rcHSV or ICP27 in the HSV stocks used to infect the BHK production cell line, since rcHSV and ICP27 may inhibit AAV production.

### REFERENCES

Adamson-Small, L., Potter, M., Falk, D. J., Cleaver, B., Byrne, B. J., and Clement, N., A scalable method for the production of high-titer and high-quality adeno-associated type 9 vectors using the HSV platform. Mol. Ther. Methods Clin. Dev. 3: 16031, 2016.
Asenbauer, B., M. McEntagart, M. D. King, P. Gallagher, M. Burke, and M. A. Farrell, Chronic active destructive herpes simplex encephalitis with recovery of viral DNA 12 years after disease onset. Neuropediatrics 29:120-123, 1998.
Booth M J, Mistry A, Li X, Thrasher A, and Coffin R S, Transfection-free and scalable recombinant AAV vector production using HSV/AAV hybrids. Gene Therapy 11:829-837, 2004.
Conway J E et al. High-titer recombinant adeno-associated virus production utilizing a recombinant herpes simplex virus type I vector expressing AAV-2 Rep and Cap. Gene Therapy 6:986-993, 1999.
Gurses, N., A. G. Kalayci, 1. Islek, and D. Us, Failure of acyclovir sodium therapy in herpes simplex encephalitis. Annals of Tropical Paediatrics. 16:173-175, 1996.
Hehir, K. M., D. Armentano, L. M. Cardoza, T. Lm Choquette, P. B. Berthelette, G. A. White, L. A. Couture, M. B. Everton, Keegan, M. Martin, D. A. Pratt, M. P. Smith, A. E. Smith, and S. C. Wadsworth, Molecular characterization of replication-competent variants of adenovirus vectors and genome modifications to prevent their occurrence. Journal of Virology. 70:8459-8467, 1996.
M K Howard, T Kershaw, B Gibb 1, N Storey, A R MacLean, B-Y Zeng, B C Tel, P Jenner, S M Brown, C J Woolf, P N Anderson, R S Coffin and D S Latchman, High efficiency gene transfer to the central nervous system of rodents and primates using herpes virus vectors lacking functional ICP27 and ICP34.5 Gene Therapy 5:1137-1147, 1998.
Kang, W., Wang, L., Harrell, H., Liu, J., Thomas, D. L., Mayfield, T. L., Scotti, M. M., Knop, D. R., and Harrell, H., Bioreactor production of recombinant herpes simplex virus vectors. Biotechnol. Prog. 23:715-721, 2007.
Penaud-Budloo, M., Francois, A., Clement, N., and Ayuso, E., Pharmacology of Recombinant Adeno-Associated Virus Production. Mol. Ther.: Med. & Clin. Dev. 8: 166-180, 2018.
Rice, S. A., and Knipe, D. M., Gene-specific transactivation by herpes simplex virus type 1 alpha protein ICP27. J. Virol. 62:3814-3823, 1988.
Rice, S. A. and Knipe, D. M., Genetic evidence for two distinct transactivation functions of the herpes simplex virus alpha protein ICP27. J. Virol. 64:1704-1715, 1990.
Rose E. Sekulovich, Kathryn Leary, T And Rozanne M. Sandri Goldin, The Herpes Simplex Virus Type 1 Protein ICP27 Can Act as a trans-Repressor or a trans-Activator in Combination with ICP4 and ICPO. J Virol 4510-4522, 1988.
Irene L. Smith, Mary Ann Hardwicke, Rozanne M. Sandri-Goldin, Evidence that the herpes simplex virus immediate early protein ICP27 acts post-transcriptionally during infection to regulate gene expression. Virology 186(1): 74-86, 1992.
Yamada S, Kameyama T, Nagaya S, et al Relapsing herpes simplex encephalitis: pathological confirmation of viral reactivation. Journal of Neurology, Neurosurgery & Psychiatry 74: 262-264, 2003.
Ye, G. J., Veres, G., and Knop, D. R., An efficient rHSV-based complementation system for the production of multiple rAAV vector serotypes. Gene Ther. 16:229-239, 2009.
Ye, G. J., Scotti, M. M., Thomas, D. L., Wang, L., Knop, D. R., and Chulay, J. D., Herpes simplex virus clearance during purification of a recombinant adeno-associated virus serotype 1 vector. Hum. Gene Ther. Clin. Dev. 25:212-217, 2014.
Zhang, W. W., P. E. Koch, and A. Roth, Detection of wildtype contamination in a recombinant adenoviral preparation by PCR. Biotechniques. 18:444-447, 1995.

All references cited herein are incorporated by reference.

DNA AND PEPTIDE SEQUENCES
(SEQ ID NO: 1)
GGATCCCAACGACCCCGCCCATGGGTCCCAATTGGCCGTCCCGTTACCAAGACCAACCCAGCCAGCGT
ATCCACCCCCGCCCGGGTCCCCGCGGAAGCGGAACGGTGTATGTGATATGCTAATTAAATACATGCCA
CGTACTTATGGTGTCTGATTGGTCCTTGTCTGTGCCGGAGGTGGGGCGGGGGCCCCGCCCGGGGGGCG
GAACTAGGAGGGGTTTGGGAGAGCCGGCCCCGGCACCACGGGTATAAGGACATCCACCACCCGGCCGG
TGGTGGTGTGCAGCCGTGTTCCAACCACGGTCACGCTTCGGTGCCTCTCCCCGATTCGGGCCCGGTCG
CTTGCTACCGGTGCGCCACCACCAGAGGCCATATCCGACACCCCAGCCCCGACGGCAGCCGACAGCCC
GGTCATGGCGACTGACATTGATATGCTAATTGACCTCGGCCTGGACCTCTCCGACAGCGATCTGGACG
AGGACCCCCCCGAGCCGGCGGAGAGCCGCCGCGACGACCTGGAATCGGACAGCAACGGGGAGTGTTCC
TCGTCGGACGAGGACATGGAAGACCCCCACGGAGAGGACGGACCGGAGCCGATACTCGACGCCGCTCG
CCCGGCGGTCCGCCCGTCTCGTCCAGAAGACCCCGGCGTACCCAGCACCCAGACGCCTCGTCCGACGG
AGCGGCAGGGCCCCAACGATCCTCAACCAGCGCCCCACAGTGTGTGGTCGCGCCTCGGGGCCCGGCGA
CCGTCTTGCTCCCCCGAGCGGCACGGGGGCAAGGTGGCCCGCCTCCAACCCCCACCGACCAAAGCCCA
GCCTGCCCGCGGCGGACGCCGTGGGCGTCGCAGGGGTCGGGGTCGCGGTGGTCCCGGGGCCGCCGATG
GTTTGTCGGACCCCCGCCGGCGTGCCCCCAGAACCAATCGCAACCCGGGGGGACCCCGCCCCGGGGCG
GGGTGGACGGACGGCCCCGGCGCCCCCCATGGCGAGGCGTGGCGCGGAAGTGAGCAGCCCGACCCACC
CGGAGGCCCGCGGACACGGAGCGTGCGCCAAGCACCCCCCCCGCTAATGACGCTGGCGATTGCCCCCC
CGCCCGCGGACCCCCGCGCCCCGGCCCCGGAGCGAAAGGCGCCCGCCGCCGACACCATCGACGCCACC
ACGCGGTTGGTCCTGCGCTCCATCTCCGAGCGCGCGGCGGTCGACCGCATCAGCGAGAGCTTCGGCCG
CAGCGCACAGGTCATGCACGACCCCTTTGGGGGGCAGCCGTTTCCCGCCGCGAATAGCCCCTGGGCCC
CGGTGCTGGCGGGCCAAGGAGGGCCCTTTGACGCCGAGACCAGACGGGTCTCCTGGGAAACCTTGGTC
GCCCACGGCCCGAGCCTCTATCGCACTTTTGCCGGCAATCCTCGGGCCGCATCGACCGCCAAGGCCAT
GCGCGACTGCGTGCTGCGCCAAGAAAATTTCATCGAGGCGCTGGCCTCCGCCGACGAGACGCTGGCGT
GGTGCAAGATGTGCATCCACCACAACCTGCCGCTGCGCCCCCAGGACCCCATTATCGGGACGGCCGCG
GCGGTGCTGGATAACCTCGCCACGCGCCTGCGGCCCTTTCTCCAGTGCTACCTGAAGGCGCGAGGCCT
GTGCGGCCTGGACGAACTGTGTTCGCGGCGGCGTCTGGCGGACATTAAGGACATTGCATCCTTCGTGT
TTGTCATTCTGGCCAGGCTCGCCAACCGCGTCGAGCGTGGCGTCGCGGAGATCGACTACGCGACCCTT
GGTGTCGGGGTCGGAGAGAAGATGCATTTCTACCTCCCCGGGGCCTGCATGGCGGGCCTGATCGAAAT
CCTAGACACGCACCGCCAGGAGTGTTCGAGTCGTGTCTGCGAGTTGACGGCCAGTCACATCGTCGCCC
CCCCGTACGTGCACGGCAAATATTTTTATTGCAACTCCCTGTTTTAGGCTAGCGAATTCCTGTGCCTT
CTAGTTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGGATGCGG
TGGGCTCTATGG
(SEQ ID NO: 2)
GGATCCCAACGACCCCGCCCATGGGTCCCAATTGGCCGTCCCGTTACCAAGACCAACCCAGCCAGCGT
ATCCACCCCCGCCCGGGTCCCCGCGGAAGCGGAACGGTGTATGTGATATGCTAATTAAATACATGCCA
CGTACTTATGGTGTCTGATTGGTCCTTGTCTGTGCCGGAGGTGGGGCGGGGGCCCCGCCCGGGGGGCG
GAACTAGGAGGGGTTTGGGAGAGCCGGCCCCGGCACCACGGGTATAAGGACATCCACCACCCGGCCGG
TGGTGGTGTGCAGCCGTGTTCCAACCACGGTCACGCTTCGGTGCCTCTCCCCGATTCGGGCCCGGTCG
CTTGCTACCGGTGCGCCACCACCAGAGGCCATATCCGACACCCCAGCCCCGACGGCAGCCGACAGCCC
GGTCATGGCGACTGACATTGATATGCTAATTGACCTCGGCCTGGACCTCTCCGACAGCGATCTGGACG
AGGACCCCCCCGAGCCGGCGGAGAGCCGCCGCGACGACCTGGAATCGGACAGCAACGGGGAGTGTTCC
TCGTCGGACGAGGACATGGAAGACCCCCACGGAGAGGACGGACCGGAGCCGATACTCGACGCCGCTCG
CCCGGCGGTCCGCCCGTCTCGTCCAGAAGACCCCGGCGTACCCAGCACCCAGACGCCTCGTCCGACGG
AGCGGCAGGGCCCCAACGATCCTCAACCAGCGCCCCACAGTGTGTGGTCGCGCCTCGGGGCCCGGCGA
CCGTCTTGCTCCCCCGAGCGGCACGGGGGCAAGGTGGCCCGCCTCCAACCCCCACCGACCAAAGCCCA
GCCTGCCCGCGGCGGACGCCGTGGGCGTCGCAGGGGTCGGGGTCGCGGTGGTCCCGGGGCCGCCGATG
GTTTGTCGGACCCCCGCCGGCGTGCCCCCAGAACCAATCGCAACCCGGGGGGACCCCGCCCCGGGGCG
GGGTGGACGGACGGCCCCGGCGCCCCCCATGGCGAGGCGTGGCGCGGAAGTGAGCAGCCCGACCCACC
CGGAGGCCCGCGGACACGGAGCGTGCGCCAAGCACCCCCCCCGCTAATGACGCTGGCGATTGCCCCCC
CGCCCGCGGACCCCCGCGCCCCGGCCCCGGAGCGAAAGGCGCCCGCCGCCGACACCATCGACGCCACC
ACGCGGTTGGTCCTGCGCTCCATCTCCGAGCGCGCGGCGGTCGACCGCATCAGCGAGAGCTTCGGCCG
CAGCGCACAGGTCATGCACGACCCCTTTGGGGGGCAGCCGTTTCCCGCCGCGAATAGCCCCTGGGCCC
CGGTGCTGGCGGGCCAAGGAGGGCCCTTTGACGCCGAGACCAGACGGGTCTCCTGGGAAACCTTGGTC
GCCCACGGCCCGAGCCTCTATCGCACTTTTGCCGGCAATCCTCGGGCCGCATCGACCGCCAAGGCCAT
GCGCGACTGCGTGCTGCGCCAAGAAAATTTCATCGAGGCGCTGGCCTCCGCCGACGAGACGCTGGCGT
GGTGCAAGATGTGCATCCACCACAACCTGCCGCTGCGCCCCCAGGACCCCATTATCGGGACGGCCGCG
GCGGTGCTGGATAACCTCGCCACGCGCCTGCGGCCCTTTCTCCAGTGCTACCTGAAGGCGCGAGGACT
CTGTGGACTCGATGAGCTCTGCTCTCGCCGACGGCTCGCCGATATCAAAGATATCGCCTCTTTTGTCT
TCGTGATCCTCGCTCGCTTGGCTAATAGAGTGGAAAGAGGAGTGGCCGAAATTGATTATGCCACATTG
GGCGTGGGCGTGGGCGAAAAAATGCACTTTTATTTACCTGGCGCTTGTATGGCTGGATTGATTGAGAT
TCTGGATACCCATAGACAAGAATGCTCCTCTAGAGTGTGTGAACTGACCGCTTCCCATATTGTGGCTC
CTCCCTATGTCCATGGAAAGTACTTCTACTGTAATAGTCTCTTCTAGGCTAGCGAATTCCTGTGCCTT
CTAGTTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGGATGCGG
TGGGCTCTATGG
(SEQ ID NO: 3)
GGATCCCAACGACCCCGCCCATGGGTCCCAATTGGCCGTCCCGTTACCAAGACCAACCCAGCCAGCGT
ATCCACCCCCGCCCGGGTCCCCGCGGAAGCGGAACGGTGTATGTGATATGCTAATTAAATACATGCCA
CGTACTTATGGTGTCTGATTGGTCCTTGTCTGTGCCGGAGGTGGGGCGGGGGCCCCGCCCGGGGGGCG
GAACTAGGAGGGGTTTGGGAGAGCCGGCCCCGGCACCACGGGTATAAGGACATCCACCACCCGGCCGG
TGGTGGTGTGCAGCCGTGTTCCAACCACGGTCACGCTTCGGTGCCTCTCCCCGATTCGGGCCCGGTCG
CTTGCTACCGGTGCGCCACCACCAGAGGCCATATCCGACACCCCAGCCCCGACGGCAGCCGACAGCCC
GGTCATGGCCACTGATATTGACATGCTTATCGACCTTGGACTCGATCTCTCCGACTCGGACTTGGATG
AGGACCCACCTGAGCCGGCCGAAAGCCGCCGGGACGATCTGGAGAGCGACTCCAACGGCGAATGCTCG
TCCTCTGACGAAGATATGGAAGATCCGCACGGCGAAGATGGCCCGGAGCCGATTCTCGACGCCGCCCG
GCCCGCCGTGCGCCCATCACGGCCTGAAGATCCCGGTGTCCCATCCACCCAAACTCCGCGGCCCACCG
AGCGCCAGGGCCCGAATGACCCCCAGCCGGCTCCGCATTCCGTGTGGAGCCGCCTGGGAGCCAGACGC
CCTTCATGCTCCCCTGAGCGGCACGGGGGAAAGGTCGCGCGGCTGCAACCTCCCCCGACCAAGGCCCA
GCCTGCCCGCGGTGGACGCCGGGGGCGCCGGAGAGGTCGCGGCAGGGGTGGCCCGGGGGCCGCAGACG
GACTGTCCGATCCGCGGCGGAGGGCGCCCAGAACGAACCGGAACCCCGGGGGCCCTAGACCTGGAGCC
GGATGGACAGACGGACCCGGAGCCCCACATGGCGAAGCGTGGAGAGGCTCAGAGCAGCCTGACCCTCC
GGGTGGCCCGAGGACCCGCAGCGTGCGGCAGGCACCACCACCCCTTATGACCCTCGCCATTGCGCCAC
CCCCGGCCGATCCGCGCGCTCCGGCCCCCGAGAGAAAGGCCCCCGCCGCCGATACCATCGATGCTACC
ACCCGCCTGGTCCTGCGGTCCATCAGCGAGAGAGCCGCAGTGGACCGCATCTCCGAATCCTTCGGCCG
GTCGGCACAGGTCATGCACGACCCGTTTGGTGGACAGCCCTTTCCTGCCGCTAACTCCCCGTGGGCAC
CCGTGCTCGCGGGACAGGGCGGCCCTTTCGACGCGGAAACCAGAAGAGTCAGCTGGGAGACTCTGGTG
GCCCACGGACCGTCCCTGTACCGGACCTTCGCCGGAAACCCGAGGGCCGCCAGCACTGCCAAGGCCAT
GCGGGACTGTGTGCTGCGCCAGGAAAACTTCATCGAAGCACTCGCCTCCGCCGACGAAACCCTGGCCT
GGTGCAAGATGTGTATTCACCATAATCTTCCTCTGCGGCCTCAAGACCCCATTATCGGGACTGCGGCG
GCCGTGTTGGACAACCTGGCGACCCGCCTGCGGCCGTTCCTGCAATGCTACCTGAAAGCCAGGGGACT
GTGCGGACTGGACGAATTGTGCTCGCGGCGCCGCCTCGCGGACATCAAGGACATCGCTTCCTTCGTGT
TCGTGATCCTGGCCAGACTCGCCAACCGAGTGGAGAGGGGAGTGGCAGAAATTGACTACGCGACTCTG
GGGGTCGGAGTGGGAGAGAAGATGCACTTCTACCTCCCTGGCGCCTGCATGGCTGGACTGATCGAGAT
CCTGGACACCCATAGACAGGAATGTTCATCCCGCGTGTGCGAGCTGACTGCGTCGCACATCGTGGCTC
CCCCCTATGTGCACGGGAAGTACTTCTACTGCAACAGCCTGTTCTAAGCTAGCGAATTCCTGTGCCTT
CTAGTTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGGATGCGG
TGGGCTCTATGG
(SEQ ID NO: 4)
GGATCCCAACGACCCCGCCCATGGGTCCCAATTGGCCGTCCCGTTACCAAGACCAACCCAGCCAGCGT
ATCCACCCCCGCCCGGGTCCCCGCGGAAGCGGAACGGTGTATGTGATATGCTAATTAAATACATGCCA
CGTACTTATGGTGTCTGATTGGTCCTTGTCTGTGCCGGAGGTGGGGCGGGGGCCCCGCCCGGGGGGCG
GAACTAGGAGGGGTTTGGGAGAGCCGGCCCCGGCACCACGGGTATAAGGACATCCACCACCCGGCCGG
TGGTGGTGTGCAGCCGTGTTCCAACCACGGTCACGCTTCGGTGCCTCTCCCCGATTCGGGCCCGGTCG
CTTGCTACCGGTGCGCCACCACCAGAGGCCATATCCGACACCCCAGCCCCGACGGCAGCCGACAGCCC
GGTCATGGCGACTGACATTGATATGCTAATTGACCTCGGCCTGGACCTCTCCGACAGCGATCTGGACG
AGGACCCCCCCGAGCCGGCGGAGAGCCGCCGCGACGACCTGGAATCGGACAGCAACGGGGAGTGTTCC
TCGTCGGACGAGGACATGGAAGACCCCCACGGAGAGGACGGACCGGAGCCGATACTCGACGCCGCTCG
CCCGGCGGTCCGCCCGTCTCGTCCAGAAGACCCCGGCGTACCCAGCACCCAGACGCCTCGTCCGACGG
AGCGGCAGGGCCCCAACGATCCTCAACCAGCGCCCCACAGTGTGTGGTCGCGCCTCGGGGCCCGGCGA
CCGTCTTGCTCCCCCGAGCGGCACGGGGGCAAGGTGGCCCGCCTCCAACCCCCACCGACCAAAGCCCA
GCCTGCCCGCGGCGGACGCCGTGGGCGTCGCAGGGGTCGGGGTCGCGGTGGTCCCGGGGCCGCCGATG
GTTTGTCGGACCCCCGCCGGCGTGCCCCCAGAACCAATCGCAACCCGGGGGGACCCCGCCCCGGGGCG
GGGTGGACGGACGGCCCCGGCGCCCCCCATGGCGAGGCGTGGCGCGGAAGTGAGCAGCCCGACCCACC
CGGAGGCCCGCGGACACGGAGCGTGCGCCAAGCACCCCCCCCGCTAATGACGCTGGCGATTGCCCCCC
CGCCCGCGGACCCCCGCGCCCCGGCCCCGGAGCGAAAGGCGCCCGCCGCCGACACCATCGACGCCACC
ACGCGGTTGGTCCTGCGCTCCATCTCCGAGCGCGCGGCGGTCGACCGCATCAGCGAGAGCTTCGGCCG
CAGCGCACAGGTCATGCACGACCCCTTTGGGGGGCAGCCGTTTCCCGCCGCGAATAGCCCCTGGGCCC
CGGTGCTGGCGGGCCAAGGAGGGCCCTTTGACGCCGAGACCAGACGGGTCTCCTGGGAAACCTTGGTC
GCCCACGGCCCGAGCCTCTATCGCACTTTTGCCGGCAATCCTCGGGCCGCATCGACCGCCAAGGCCAT
GCGCGACTGCGTGCTGCGCCAAGAAAATTTCATCGAGGCGCTGGCCTCCGCCGACGAGACGCTGGCGT
GGTGCAAGATGTGCATCCACCACAACCTGCCGCTGCGCCCCCAGGACCCCATTATCGGGACGGCCGCG
GCGGTGCTGGATAACCTCGCCACGCGCCTGCGGCCCTTTCTCCAGTGCTACCTGAAGGCGCGAGGCCT
GTGCGGCCTGGACGAACTGTGTTCGCGGCGGCGTCTGGCGGACATTAAGGACATTGCATCCTTCGTGT
TTGTCATTCTGGCCAGGCTCGCCAACCGCGTCGAGCGTGGCGTCGCGGAGATCGACTACGCGACCCTT
GGTGTCGGGGTCGGAGAGAAGATGCATTTCTACCTCCCCGGGGCCTGCATGGCGGGCCTGATCGAAAT
CCTAGACACGCACCGCCAGGAGTGTTCGAGTCGTGTCTGCGAGTTGACGGCCAGTCACATCGTCGCCC
CCCCGTACGTGCACGGCAAATATTTTTATTGCAACTCCCTGTTTTAGGTACAATAAAAACAAAACATT
TCAAACAAATCGCCCCACGTGTTGTCCTTCTTTGCTCATGGCCGGCGGGGCGTGGGTCACGGCAGATG
GCGGGGGTGGGCCCGGCGTACGGCCTGGGTGGGCGGAGGGAACTAACCCAACGTATAAATCCGTCCCC
GCTCCAAGGCCGGTGTCATAGTGCCCTTAGGAGCTTCCCGCCCGGGCGCATCCCCCCTTTTGCACTAT
GACAGCGACCCCCCTCACCAACCTGTTCTTACGGGCCCCGGACATAACCCACGTGGCCCCCCCTTACT
GCCTCAACGCCACCTGGCAGGCCGAAACGGCCATGCACACCAGCAAAACGGACTCCGCTTGCGTGGCC
GTGCGGAGTTACCTGGTCCGCGCCTCCTGTGAGACCAGCGGCACAATCCACTGCTTTTTCTTTGCGGT
ATACAAGGACACCCACCATACCCCTCCGCTGATTACCGAGCTCCGCAACTTTGCGGACCTGGTT
(SEQ ID NO: 5)
CGTCACATCCAGGCCGGCGGAAACCGGAACGGCATATGCAAACTGGAAACTGTCCTGTCTTGGGGCCC
ACCCACCCGACGCGTCATATGTAAATGAAAATCGTTCCCCCGAGGCCATGTGTAGCCTGGATCCCAAC
GACCCCGCCCATGGGTCCCAATTGGCCGTCCCGTTACCAAGACCAACCCAGCCAGCGTATCCACCCCC
GCCCGGGTCCCCGCGGAAGCGGAACGGTGTATGTGATATGCTAATTAAATACATGCCACGTACTTATG
GTGTCTGATTGGTCCTTGTCTGTGCCGGAGGTGGGGCGGGGGCCCCGCCCGGGGGGCGGAACTAGGAG
GGGTTTGGGAGAGCCGGCCCCGGCACCACGGGTATAAGGACATCCACCACCCGGCCGGTGGTGGTGTG
CAGCCGTGTTCCAACCACGGTCACGCTTCGGTGCCTCTCCCCGATTCGGGCCCGGTCGCTTGCTACCG
GTGCGCCACCACCAGAGGCCATATCCGACACCCCAGCCCCGACGGCAGCCGACAGCCCGGTCATGGCG
ACTGACATTGATATGCTAATTGACCTCGGCCTGGACCTCTCCGACAGCGATCTGGACGAGGACCCCCC
CGAGCCGGCGGAGAGCCGCCGCGACGACCTGGAATCGGACAGCAACGGGGAGTGTTCCTCGTCGGACG
AGGACATGGAAGACCCCCACGGAGAGGACGGACCGGAGCCGATACTCGACGCCGCTCGCCCGGCGGTC
CGCCCGTCTCGTCCAGAAGACCCCGGCGTACCCAGCACCCAGACGCCTCGTCCGACGGAGCGGCAGGG
CCCCAACGATCCTCAACCAGCGCCCCACAGTGTGTGGTCGCGCCTCGGGGCCCGGCGACCGTCTTGCT
CCCCCGAGCGGCACGGGGGCAAGGTGGCCCGCCTCCAACCCCCACCGACCAAAGCCCAGCCTGCCCGC
GGCGGACGCCGTGGGCGTCGCAGGGGTCGGGGTCGCGGTGGTCCCGGGGCCGCCGATGGTTTGTCGGA
CCCCCGCCGGCGTGCCCCCAGAACCAATCGCAACCCGGGGGGACCCCGCCCCGGGGCGGGGTGGACGG
ACGGCCCCGGCGCCCCCCATGGCGAGGCGTGGCGCGGAAGTGAGCAGCCCGACCCACCCGGAGGCCCG
CGGACACGGAGCGTGCGCCAAGCACCCCCCCCGCTAATGACGCTGGCGATTGCCCCCCCGCCCGCGGA
CCCCCGCGCCCCGGCCCCGGAGCGAAAGGCGCCCGCCGCCGACACCATCGACGCCACCACGCGGTTGG
TCCTGCGCTCCATCTCCGAGCGCGCGGCGGTCGACCGCATCAGCGAGAGCTTCGGCCGCAGCGCACAG
GTCATGCACGACCCCTTTGGGGGGCAGCCGTTTCCCGCCGCGAATAGCCCCTGGGCCCCGGTGCTGGC
GGGCCAAGGAGGGCCCTTTGACGCCGAGACCAGACGGGTCTCCTGGGAAACCTTGGTCGCCCACGGCC
CGAGCCTCTATCGCACTTTTGCCGGCAATCCTCGGGCCGCATCGACCGCCAAGGCCATGCGCGACTGC
GTGCTGCGCCAAGAAAATTTCATCGAGGCGCTGGCCTCCGCCGACGAGACGCTGGCGTGGTGCAAGAT
GTGCATCCACCACAACCTGCCGCTGCGCCCCCAGGACCCCATTATCGGGACGGCCGCGGCGGTGCTGG
ATAACCTCGCCACGCGCCTGCGGCCCTTTCTCCAGTGCTACCTGAAGGCGCGAGGACTCTGTGGACTC
GATGAGCTCTGCTCTCGCCGACGGCTCGCCGATATCAAAGATATCGCCTCTTTTGTCTTCGTGATCCT
CGCTCGCTTGGCTAATAGAGTGGAAAGAGGAGTGGCCGAAATTGATTATGCCACATTGGGCGTGGGCG
TGGGCGAAAAAATGCACTTTTATTTACCTGGCGCTTGTATGGCTGGATTGATTGAGATTCTGGATACC
CATAGACAAGAATGCTCCTCTAGAGTGTGTGAACTGACCGCTTCCCATATTGTGGCTCCTCCCTATGT
CCATGGAAAGTACTTCTACTGTAATAGTCTCTTCTAGGCTAGCGAATTCCTGTGCCTTCTAGTTGCCA
GCCATCTGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGCGGTGGGCTCTAT
GG
(SEQ ID NO: 6)
GGATCCCAACGACCCCGCCCATGGGTCCCAATTGGCCGTCCCGTTACCAAGACCAACCCAGCCAGCGT
ATCCACCCCCGCCCGGGTCCCCGCGGAAGCGGAACGGTGTATGTGATATGCTAATTAAATACATGCCA
CGTACTTATGGTGTCTGATTGGTCCTTGTCTGTGCCGGAGGTGGGGCGGGGGCCCCGCCCGGGGGGCG
GAACTAGGAGGGGTTTGGGAGAGCCGGCCCCGGCACCACGGGTATAAGGACATCCACCACCCGGCCGG
TGGTGGTGTGCAGCCGTGTTCCAACCACGGTCACGCTTCGGTGCCTCTCCCCGATTCGGGCCCGGTCG
CTTGCTACCGGTGCGCCACCACCAGAGGCCATATCCGACACCCCAGCCCCGACGGCAGCCGACAGCCC
GGTCATGGCCACCGACATCGACATGCTGATCGATTTAGGTTTAGATTTAAGCGACAGCGATCTGGACG
AGGACCCCCCCGAACCCGCTGAATCTCGTCGGGACGATTTAGAGTCCGACTCCAATGGAGAGTGTAGC
AGCAGCGACGAGGACATGGAAGACCCTCACGGCGAGGATGGCCCCGAACCTATTCTCGATGCTGCTCG
GCCCGCTGTGAGGCCTTCCCGGCCCGAAGATCCCGGTGTGCCTAGCACCCAAACCCCTCGTCCTACCG
AGCGGCAAGGTCCCAATGATCCTCAGCCCGCCCCTCATAGCGTCTGGTCTCGTCTGGGAGCTAGGAGG
CCTAGCTGTTCCCCCGAACGGCACGGAGGCAAAGTGGCTAGGCTGCAGCCTCCCCCCACCAAAGCTCA
ACCCGCTCGGGGAGGCCGGAGGGGTCGTCGGCGGGGTCGTGGAAGGGGCGGCCCCGGTGCTGCCGACG
GACTGAGCGATCCTAGGAGGAGGGCCCCTCGGACCAATCGTAATCCCGGTGGACCTCGTCCCGGTGCT
GGATGGACCGATGGACCCGGTGCTCCTCACGGAGAGGCTTGGAGGGGAAGCGAGCAGCCCGATCCTCC
CGGTGGCCCTAGGACAAGGAGCGTTCGTCAAGCTCCTCCTCCTCTCATGACTTTAGCCATTGCCCCTC
CTCCCGCTGATCCTAGGGCTCCCGCTCCCGAAAGGAAAGCCCCCGCCGCCGATACCATTGACGCCACA
ACTCGTCTCGTGCTGAGGTCCATTTCCGAACGGGCCGCCGTCGATCGTATCAGCGAGAGCTTCGGAAG
GTCCGCCCAAGTTATGCACGATCCCTTCGGCGGCCAACCCTTTCCCGCTGCTAATAGCCCTTGGGCCC
CCGTGCTGGCTGGACAAGGAGGCCCTTTCGACGCCGAGACTCGTAGGGTGAGCTGGGAGACACTGGTG
GCCCATGGCCCTTCTTTATACCGGACATTCGCTGGCAACCCTCGTGCTGCCAGCACAGCTAAGGCCAT
GCGGGACTGTGTGCTGCGGCAAGAAAACTTCATTGAGGCTTTAGCCAGCGCTGATGAGACTTTAGCTT
GGTGCAAGATGTGCATCCACCACAATTTACCTCTGAGGCCCCAAGATCCCATCATTGGCACAGCCGCC
GCCGTGCTGGATAATTTAGCCACTCGTCTCCGGCCCTTTCTGCAGTGCTACCTCAAAGCCCGGGGTTT
ATGCGGACTCGATGAGCTGTGTTCTCGTCGGAGGCTGGCCGACATCAAGGACATCGCCAGCTTCGTGT
TCGTGATCCTCGCTCGTCTGGCCAATCGTGTGGAGAGGGGAGTGGCCGAAATCGATTATGCCACCTTA
GGCGTGGGCGTTGGCGAGAAGATGCACTTTTATTTACCCGGTGCTTGTATGGCCGGACTCATTGAGAT
CCTCGATACCCACCGGCAAGAATGCTCCTCTCGTGTGTGCGAGCTGACCGCTTCCCACATTGTGGCCC
CCCCCTACGTGCACGGAAAATACTTCTACTGTAACTCTTTATTCTGAGCTAGCGAATTCCTGTGCCTT
CTAGTTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGGATGCGG
TGGGCTCTATGG
(SEQ ID NO: 7)
GGATCCCAACGACCCCGCCCATGGGTCCCAATTGGCCGTCCCGTTACCAAGACCAACCCAGCCAGCGT
ATCCACCCCCGCCCGGGTCCCCGCGGAAGCGGAACGGTGTATGTGATATGCTAATTAAATACATGCCA
CGTACTTATGGTGTCTGATTGGTCCTTGTCTGTGCCGGAGGTGGGGCGGGGGCCCCGCCCGGGGGGCG
GAACTAGGAGGGGTTTGGGAGAGCCGGCCCCGGCACCACGGGTATAAGGACATCCACCACCCGGCCGG
TGGTGGTGTGCAGCCGTGTTCCAACCACGGTCACGCTTCGGTGCCTCTCCCCGATTCGGGCCCGGTCG
CTTGCTACCGGTGCGCCACCACCAGAGGCCATATCCGACACCCCAGCCCCGACGGCAGCCGACAGCCC
GGTCATGGCTACAGACATCGACATGCTGATCGACCTGGGCCTCGACCTGTCTGACTCTGATCTGGACG
AAGATCCTCCTGAGCCTGCCGAGTCCAGAAGGGACGACCTGGAATCTGACTCTAACGGCGAGTGCTCC
TCCAGCGACGAGGATATGGAAGATCCACATGGCGAGGACGGCCCTGAGCCTATTTTGGATGCTGCCAG
ACCTGCCGTGCGGCCTTCTAGACCTGAAGATCCTGGCGTGCCATCTACACAGACCCCTAGACCTACAG
AGCGGCAGGGCCCTAATGATCCTCAGCCTGCTCCTCACTCCGTGTGGTCTAGATTGGGAGCCAGACGG
CCTTCCTGCTCTCCTGAAAGACATGGCGGCAAGGTGGCAAGACTGCAGCCTCCTCCAACAAAGGCTCA
ACCTGCTAGAGGCGGCAGAAGGGGCAGACGTAGAGGTAGAGGAAGAGGTGGACCTGGCGCTGCTGATG
GCTTGTCTGATCCTAGAAGAAGGGCCCCTCGGACCAACAGAAATCCTGGTGGACCTAGACCAGGCGCC
GGATGGACAGATGGTCCAGGTGCTCCTCATGGCGAAGCTTGGAGAGGATCTGAGCAACCTGATCCTCC
AGGCGGCCCTAGAACCAGATCTGTTAGACAGGCTCCTCCTCCTCTGATGACCCTGGCTATTGCTCCAC
CTCCTGCCGATCCTAGAGCACCCGCTCCAGAAAGAAAGGCCCCTGCTGCTGACACCATCGACGCTACA
ACAAGACTGGTGCTGCGGTCCATCTCTGAGAGGGCCGCTGTGGACAGAATCTCCGAGTCTTTCGGCCG
CTCTGCCCAAGTGATGCACGATCCTTTTGGCGGCCAGCCTTTTCCTGCCGCCAATTCTCCTTGGGCTC
CTGTGTTGGCTGGCCAAGGCGGACCTTTTGACGCCGAGACAAGAAGAGTGTCCTGGGAGACACTGGTG
GCTCACGGACCTAGCCTGTACAGAACCTTCGCTGGCAACCCTAGAGCCGCTTCTACCGCCAAGGCCAT
GAGAGACTGTGTGCTGAGACAAGAGAACTTCATCGAGGCCCTGGCCTCTGCCGATGAGACTCTGGCTT
GGTGCAAGATGTGTATCCACCACAACCTGCCTCTGCGGCCTCAGGACCCTATCATTGGAACAGCTGCC
GCCGTGCTGGATAACCTGGCTACCAGACTCAGACCCTTCCTGCAGTGCTACCTGAAGGCTAGAGGCCT
GTGTGGCCTGGACGAGCTGTGCTCCAGAAGAAGGCTGGCTGATATCAAGGATATCGCCTCCTTCGTGT
TCGTGATCCTGGCCAGACTGGCTAACAGAGTGGAAAGAGGCGTGGCCGAGATCGATTATGCTACCCTC
GGAGTCGGCGTGGGCGAGAAGATGCATTTTTACCTGCCTGGCGCCTGCATGGCCGGACTGATCGAGAT
TCTGGATACCCACCGGCAAGAGTGCTCCAGCAGAGTGTGTGAACTGACCGCCTCTCACATCGTGGCTC
CTCCATACGTGCACGGCAAGTACTTCTACTGCAACTCCCTGTTCTGAGCTAGCGAATTCCTGTGCCTT
CTAGTTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGGATGCGG
TGGGCTCTATGG
(SEQ ID NO: 8)
CGTCACATCCAGGCCGGCGGAAACCGGAACGGCATATGCAAACTGGAAACTGTCCTGTCTTGGGGCCC
ACCCACCCGACGCGTCATATGTAAATGAAAATCGTTCCCCCGAGGCCATGTGTAGCCTGGATCCCAAC
GACCCCGCCCATGGGTCCCAATTGGCCGTCCCGTTACCAAGACCAACCCAGCCAGCGTATCCACCCCC
GCCCGGGTCCCCGCGGAAGCGGAACGGTGTATGTGATATGCTAATTAAATACATGCCACGTACTTATG
GTGTCTGATTGGTCCTTGTCTGTGCCGGAGGTGGGGCGGGGGCCCCGCCCGGGGGGCGGAACTAGGAG
GGGTTTGGGAGAGCCGGCCCCGGCACCACGGGTATAAGGACATCCACCACCCGGCCGGTGGTGGTGTG
CAGCCGTGTTCCAACCACGGTCACGCTTCGGTGCCTCTCCCCGATTCGGGCCCGGTCGCTTGCTACCG
GTGCGCCACCACCAGAGGCCATATCCGACACCCCAGCCCCGACGGCAGCCGACAGCCCGGTCATGGCG
ACTGACATTGATATGCTAATTGACCTCGGCCTGGACCTCTCCGACAGCGATCTGGACGAGGACCCCCC
CGAGCCGGCGGAGAGCCGCCGCGACGACCTGGAATCGGACAGCAACGGGGAGTGTTCCTCGTCGGACG
AGGACATGGAAGACCCCCACGGAGAGGACGGACCGGAGCCGATACTCGACGCCGCTCGCCCGGCGGTC
CGCCCGTCTCGTCCAGAAGACCCCGGCGTACCCAGCACCCAGACGCCTCGTCCGACGGAGCGGCAGGG
CCCCAACGATCCTCAACCAGCGCCCCACAGTGTGTGGTCGCGCCTCGGGGCCCGGCGACCGTCTTGCT
CCCCCGAGCGGCACGGGGGCAAGGTGGCCCGCCTCCAACCCCCACCGACCAAAGCCCAGCCTGCCCGC
GGCGGACGCCGTGGGCGTCGCAGGGGTCGGGGTCGCGGTGGTCCCGGGGCCGCCGATGGTTTGTCGGA
CCCCCGCCGGCGTGCCCCCAGAACCAATCGCAACCCGGGGGGACCCCGCCCCGGGGCGGGGTGGACGG
ACGGCCCCGGCGCCCCCCATGGCGAGGCGTGGCGCGGAAGTGAGCAGCCCGACCCACCCGGAGGCCCG
CGGACACGGAGCGTGCGCCAAGCACCCCCCCCGCTAATGACGCTGGCGATTGCCCCCCCGCCCGCGGA
CCCCCGCGCCCCGGCCCCGGAGCGAAAGGCGCCCGCCGCCGACACCATCGACGCCACCACGCGGTTGG
TCCTGCGCTCCATCTCCGAGCGCGCGGCGGTCGACCGCATCAGCGAGAGCTTCGGCCGCAGCGCACAG
GTCATGCACGACCCCTTTGGGGGGCAGCCGTTTCCCGCCGCGAATAGCCCCTGGGCCCCGGTGCTGGC
GGGCCAAGGAGGGCCCTTTGACGCCGAGACCAGACGGGTCTCCTGGGAAACCTTGGTCGCCCACGGCC
CGAGCCTCTATCGCACTTTTGCCGGCAATCCTCGGGCCGCATCGACCGCCAAGGCCATGCGCGACTGC
GTGCTGCGCCAAGAAAATTTCATCGAGGCGCTGGCCTCCGCCGACGAGACGCTGGCGTGGTGCAAGAT
GTGCATCCACCACAACCTGCCGCTGCGCCCCCAGGACCCCATTATCGGGACGGCCGCGGCGGTGCTGG
ATAACCTCGCCACGCGCCTGCGGCCCTTTCTCCAGTGCTACCTGAAGGCGCGAGGCCTGTGCGGCCTG
GACGAACTGTGTTCGCGGCGGCGTCTGGCGGACATTAAGGACATTGCATCCTTCGTGTTTGTCATTCT
GGCCAGGCTCGCCAACCGCGTCGAGCGTGGCGTCGCGGAGATCGACTACGCGACCCTTGGTGTCGGGG
TCGGAGAGAAGATGCATTTCTACCTCCCCGGGGCCTGCATGGCGGGCCTGATCGAAATCCTAGACACG
CACCGCCAGGAGTGTTCGAGTCGTGTCTGCGAGTTGACGGCCAGTCACATCGTCGCCCCCCCGTACGT
GCACGGCAAATATTTTTATTGCAACTCCCTGTTTTAGGCTAGCGAATTCCTGTGCCTTCTAGTTGCCA
GCCATCTGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGCGGTGGGCTCTAT
GG
(SEQ ID NO: 9)
GGATCCCAACGACCCCGCCCATGGGTCCCAATTGGCCGTCCCGTTACCAAGACCAACCCAGCCAGCGT
ATCCACCCCCGCCCGGGTCCCCGCGGAAGCGGAACGGTGTATGTGATATGCTAATTAAATACATGCCA
CGTACTTATGGTGTCTGATTGGTCCTTGTCTGTGCCGGAGGTGGGGCGGGGGCCCCGCCCGGGGGGCG
GAACTAGGAGGGGTTTGGGAGAGCCGGCCCCGGCACCACGGGTATAAGGACATCCACCACCCGGCCGG
TGGTGGTGTGCAGCCGTGTTCCAACCACGGTCACGCTTCGGTGCCTCTCCCCGATTCGGGCCCGGTCG
CTTGCTACCGGTGCGCCACCACCAGAGGCCATATCCGACACCCCAGCCCCGACGGCAGCCGACAGCCC
GGTCATGGCGACTGACATTGATATGCTAATTGACCTCGGCCTGGACCTCTCCGACAGCGATCTGGACG
AGGACCCCCCCGAGCCGGCGGAGAGCCGCCGCGACGACCTGGAATCGGACAGCAACGGGGAGTGTTCC
TCGTCGGACGAGGACATGGAAGACCCCCACGGAGAGGACGGACCGGAGCCGATACTCGACGCCGCTCG
CCCGGCGGTCCGCCCGTCTCGTCCAGAAGACCCCGGCGTACCCAGCACCCAGACGCCTCGTCCGACGG
AGCGGCAGGGCCCCAACGATCCTCAACCAGCGCCCCACAGTGTGTGGTCGCGCCTCGGGGCCCGGCGA
CCGTCTTGCTCCCCCGAGCGGCACGGGGGCAAGGTGGCCCGCCTCCAACCCCCACCGACCAAAGCCCA
GCCTGCCCGCGGCGGACGCCGTGGGCGTCGCAGGGGTCGGGGTCGCGGTGGTCCCGGGGCCGCCGATG
GTTTGTCGGACCCCCGCCGGCGTGCCCCCAGAACCAATCGCAACCCGGGGGGACCCCGCCCCGGGGCG
GGGTGGACGGACGGCCCCGGCGCCCCCCATGGCGAGGCGTGGCGCGGAAGTGAGCAGCCCGACCCACC
CGGAGGCCCGCGGACACGGAGCGTGCGCCAAGCACCCCCCCCGCTAATGACGCTGGCGATTGCCCCCC
CGCCCGCGGACCCCCGCGCCCCGGCCCCGGAGCGAAAGGCGCCCGCCGCCGACACCATCGACGCCACC
ACGCGGTTGGTCCTGCGCTCCATCTCCGAGCGCGCGGCGGTCGACCGCATCAGCGAGAGCTTCGGCCG
CAGCGCACAGGTCATGCACGACCCCTTTGGGGGGCAGCCGTTTCCCGCCGCGAATAGCCCCTGGGCCC
CGGTGCTGGCGGGCCAAGGAGGGCCCTTTGACGCCGAGACCAGACGGGTCTCCTGGGAAACCTTGGTC
GCCCACGGCCCGAGCCTCTATCGCACTTTTGCCGGCAATCCTCGGGCCGCATCGACCGCCAAGGCCAT
GCGCGACTGCGTGCTGCGCCAAGAAAATTTCATCGAGGCGCTGGCCTCCGCCGACGAGACGCTGGCGT
GGTGCAAGATGTGCATCCACCACAACCTGCCGCTGCGCCCCCAGGACCCCATTATCGGGACGGCCGCG
GCGGTGCTGGATAACCTCGCCACGCGCCTGCGGCCCTTTCTCCAGTGCTACCTGAAGGCGCGAGGCCT
GTGCGGCCTGGATGAACTGTGCTCTAGAAGAAGGCTGGCCGATATCAAGGATATCGCCTCCTTCGTGT
TCGTGATCCTGGCTCGGCTGGCCAACAGAGTGGAAAGAGGCGTGGCCGAGATCGACTATGCTACCCTC
GGAGTTGGCGTGGGCGAGAAGATGCACTTTTACCTGCCTGGCGCCTGTATGGCCGGCCTGATCGAGAT
CCTGGACACCCACAGACAAGAGTGCTCCTCCAGAGTGTGCGAGCTGACCGCTTCTCACATCGTGGCTC
CTCCATACGTGCACGGCAAGTACTTCTACTGCAACTCCCTGTTCTGAGCTAGCGAATTCCTGTGCCTT
CTAGTTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGGATGCGG
TGGGCTCTATGG
(SEQ ID NO: 10)
MATDIDMLIDLGLDLSDSDLDEDPPEPAESRRDDLESDSNGECSSSDEDMEDPHGEDGPEPILDAARP
AVRPSRPEDPGVPSTQTPRPTERQGPNDPQPAPHSVWSRLGARRPSCSPERHGGKVARLQPPPTKAQP
ARGGRRGRRRGRGRGGPGAADGLSDPRRRAPRTNRNPGGPRPGAGWTDGPGAPHGEAWRGSEQPDPPG
GPRIRSVRQAPPPLMTLAIAPPPADPRAPAPERKAPAADTIDATTRLVLRSISERAAVDRISESFGRS
AQVMHDPFGGQPFPAANSPWAPVLAGQGGPFDAETRRVSWEILVAHGPSLYRTFAGNPRAASTAKAMR
DCVLRQENFIEALASADETLAWCKMCIHHNLPLRPQDPIIGTAAAVLDNLATRLRPFLQCYLKARGLC
GLDELCSRRRLADIKDIASFVFVILARLANRVERGVAEIDYATLGVGVGEKMHFYLPGACMAGLIEIL
DTHRQECSSRVCELTASHIVAPPYVHGKYFYCNSLF*
(SEQ ID NO: 11)
CGTCACATCCAGGCCGGCGGAAACCGGAACGGCATATGCAAACTGGAAACTGTCCTGTCTTGGGGCCC
ACCCACCCGACGCGTCATATGTAAATGAAAATCGTTCCCCCGAGGCCATGTGTAGCCTGGATCCCAAC
GACCCCGCCCATGGGTCCCAATTGGCCGTCCCGTTACCAAGACCAACCCAGCCAGCGTATCCACCCCC
GCCCGGGTCCCCGCGGAAGCGGAACGGTGTATGTGATATGCTAATTAAATACATGCCACGTACTTATG
GTGTCTGATTGGTCCTTGTCTGTGCCGGAGGTGGGGCGGGGGCCCCGCCCGGGGGGCGGAACTAGGAG
GGGTTTGGGAGAGCCGGCCCCGGCACCACGGGTATAAGGACATCCACCACCCGGCCGGTGGTGGTGTG
CAGCCGTGTTCCAACCACGGTCACGCTTCGGTGCCTCTCCCCGATTCGGGCCCGGTCGCTTGCTACCG
GTGCGCCACCACCAGAGGCCATATCCGACACCCCAGCCCCGACGGCAGCCGACAGCCCGGTCATGGCG
ACTGACATTGATATGCTAATTGACCTCGGCCTGGACCTCTCCGACAGCGATCTGGACGAGGACCCCCC
CGAGCCGGCGGAGAGCCGCCGCGACGACCTGGAATCGGACAGCAACGGGGAGTGTTCCTCGTCGGACG
AGGACATGGAAGACCCCCACGGAGAGGACGGACCGGAGCCGATACTCGACGCCGCTCGCCCGGCGGTC
CGCCCGTCTCGTCCAGAAGACCCCGGCGTACCCAGCACCCAGACGCCTCGTCCGACGGAGCGGCAGGG
CCCCAACGATCCTCAACCAGCGCCCCACAGTGTGTGGTCGCGCCTCGGGGCCCGGCGACCGTCTTGCT
CCCCCGAGCGGCACGGGGGCAAGGTGGCCCGCCTCCAACCCCCACCGACCAAAGCCCAGCCTGCCCGC
GGCGGACGCCGTGGGCGTCGCAGGGGTCGGGGTCGCGGTGGTCCCGGGGCCGCCGATGGTTTGTCGGA
CCCCCGCCGGCGTGCCCCCAGAACCAATCGCAACCCGGGGGGACCCCGCCCCGGGGCGGGGTGGACGG
ACGGCCCCGGCGCCCCCCATGGCGAGGCGTGGCGCGGAAGTGAGCAGCCCGACCCACCCGGAGGCCCG
CGGACACGGAGCGTGCGCCAAGCACCCCCCCCGCTAATGACGCTGGCGATTGCCCCCCCGCCCGCGGA
CCCCCGCGCCCCGGCCCCGGAGCGAAAGGCGCCCGCCGCCGACACCATCGACGCCACCACGCGGTTGG
TCCTGCGCTCCATCTCCGAGCGCGCGGCGGTCGACCGCATCAGCGAGAGCTTCGGCCGCAGCGCACAG
GTCATGCACGACCCCTTTGGGGGGCAGCCGTTTCCCGCCGCGAATAGCCCCTGGGCCCCGGTGCTGGC
GGGCCAAGGAGGGCCCTTTGACGCCGAGACCAGACGGGTCTCCTGGGAAACCTTGGTCGCCCACGGCC
CGAGCCTCTATCGCACTTTTGCCGGCAATCCTCGGGCCGCATCGACCGCCAAGGCCATGCGCGACTGC
GTGCTGCGCCAAGAAAATTTCATCGAGGCGCTGGCCTCCGCCGACGAGACGCTGGCGTGGTGCAAGAT
GTGCATCCACCACAACCTGCCGCTGCGCCCCCAGGACCCCATTATCGGGACGGCCGCGGCGGTGCTGG
ATAACCTCGCCACGCGCCTGCGGCCCTTTCTCCAGTGCTACCTGAAGGCGCGAGGCCTGTGCGGCCTG
GACGAACTGTGTTCGCGGCGGCGTCTGGCGGACATTAAGGACATTGCATCCTTCGTGTTTGTCATTCT
GGCCAGGCTCGCCAACCGCGTCGAGCGTGGCGTCGCGGAGATCGACTACGCGACCCTTGGTGTCGGGG
TCGGAGAGAAGATGCATTTCTACCTCCCCGGGGCCTGCATGGCGGGCCTGATCGAAATCCTAGACACG
CACCGCCAGGAGTGTTCGAGTCGTGTCTGCGAGTTGACGGCCAGTCACATCGTCGCCCCCCCGTACGT
GCACGGCAAATATTTTTATTGCAACTCCCTGTTTTAG
(SEQ ID NO: 12)
CCACCCCCGCCCGGGTCCCCGCGGAAGCGGAACGGTGTATGTGATATGCTAATTAAATACATGCCACG
TACTTATGGTGTCTGATTGGTCCTTGTCTGTGCCGGAGGTGGGGCGGGGGCCCCGCCCGGGGGGCGGA
ACTAGGAGGGGTTTGGGAGAGCCGGCCCCGGCACCACGGGTATAAGGACATCCACCACCCGGCCGGTG
GTGGTGTGCAGCCGTGTTCCAACCACGGTCACGCTTCGGTGCCTCTCCCCGATTCGGGCCCGGTCGCT
TGCTACCGGTGCGCCACCACCAGAGGCCATATCCGACACCCCAGCCCCGACGGCAGCCGACAGCCCGG
TCATGGCGACTGACATTGATATGCTAATTGACCTCGGCCTGGACCTCTCCGACAGCGATCTGGACGAG
GACCCCCCCGAGCCGGCGGAGAGCCGCCGCGACGACCTGGAATCGGACAGCAACGGGGAGTGTTCCTC
GTCGGACGAGGACATGGAAGACCCCCACGGAGAGGACGGACCGGAGCCGATACTCGACGCCGCTCGCC
CGGCGGTCCGCCCGTCTCGTCCAGAAGACCCCGGCGTACCCAGCACCCAGACGCCTCGTCCGACGGAG
CGGCAGGGCCCCAACGATCCTCAACCAGCGCCCCACAGTGTGTGGTCGCGCCTCGGGGCCCGGCGACC
GTCTTGCTCCCCCGAGCGGCACGGGGGCAAGGTGGCCCGCCTCCAACCCCCACCGACCAAAGCCCAGC
CTGCCCGCGGCGGACGCCGTGGGCGTCGCAGGGGTCGGGGTCGCGGTGGTCCCGGGGCCGCCGATGGT
TTGTCGGACCCCCGCCGGCGTGCCCCCAGAACCAATCGCAACCCGGGGGGACCCCGCCCCGGGGCGGG
GTGGACGGACGGCCCCGGCGCCCCCCATGGCGAGGCGTGGCGCGGAAGTGAGCAGCCCGACCCACCCG
GAGGCCCGCGGACACGGAGCGTGCGCCAAGCACCCCCCCCGCTAATGACGCTGGCGATTGCCCCCCCG
CCCGCGGACCCCCGCGCCCCGGCCCCGGAGCGAAAGGCGCCCGCCGCCGACACCATCGACGCCACCAC
GCGGTTGGTCCTGCGCTCCATCTCCGAGCGCGCGGCGGTCGACCGCATCAGCGAGAGCTTCGGCCGCA
GCGCACAGGTCATGCACGACCCCTTTGGGGGGCAGCCGTTTCCCGCCGCGAATAGCCCCTGGGCCCCG
GTGCTGGCGGGCCAAGGAGGGCCCTTTGACGCCGAGACCAGACGGGTCTCCTGGGAAACCTTGGTCGC
CCACGGCCCGAGCCTCTATCGCACTTTTGCCGGCAATCCTCGGGCCGCATCGACCGCCAAGGCCATGC
GCGACTGCGTGCTGCGCCAAGAAAATTTCATCGAGGCGCTGGCCTCCGCCGACGAGACGCTGGCGTGG
TGCAAGATGTGCATCCACCACAACCTGCCGCTGCGCCCCCAGGACCCCATTATCGGGACGGCCGCGGC
GGTGCTGGATAACCTCGCCACGCGCCTGCGGCCCTTTCTCCAGTGCTACCTGAAGGCGCGAGGACTCT
GTGGACTCGATGAGCTCTGCTCTCGCCGACGGCTCGCCGATATCAAAGATATCGCCTCTTTTGTOTTC
GTGATCCTCGCTCGCTTGGCTAATAGAGTGGAAAGAGGAGTGGCCGAAATTGATTATGCCACATTGGG
CGTGGGCGTGGGCGAAAAAATGCACTTTTATTTACCTGGCGCTTGTATGGCTGGATTGATTGAGATTC
TGGATACCCATAGACAAGAATGCTCCTCTAGAGTGTGTGAACTGACCGCTTCCCATATTGTGGCTCCT
CCCTATGTCCATGGAAAGTACTTCTACTGTAATAGTCTCTTCTAG
(SEQ ID NO: 13)
GGATCCCAACGACCCCGCCCATGGGTCCCAATTGGCCGTCCCGTTACCAAGACCAACCCAGCCAGCGT
ATCCACCCCCGCCCGGGTCCCCGCGGAAGCGGAACGGTGTATGTGATATGCTAATTAAATACATGCCA
CGTACTTATGGTGTCTGATTGGTCCTTGTCTGTGCCGGAGGTGGGGCGGGGGCCCCGCCCGGGGGGCG
GAACTAGGAGGGGTTTGGGAGAGCCGGCCCCGGCACCACGGGTATAAGGACATCCACCACCCGGCCGG
TGGTGGTGTGCAGCCGTGTTCCAACCACGGTCACGCTTCGGTGCCTCTCCCCGATTCGGGCCCGGTCG
CTTGCTACCGGTGCGCCACCACCAGAGGCCATATCCGACACCCCAGCCCCGACGGCAGCCGACAGCCC
GGTCATGGCGACTGACATTGATATGCTAATTGACCTCGGCCTGGACCTCTCCGACAGCGATCTGGACG
AGGACCCCCCCGAGCCGGCGGAGAGCCGCCGCGACGACCTGGAATCGGACAGCAACGGGGAGTGTTCC
TCGTCGGACGAGGACATGGAAGACCCCCACGGAGAGGACGGACCGGAGCCGATACTCGACGCCGCTCG
CCCGGCGGTCCGCCCGTCTCGTCCAGAAGACCCCGGCGTACCCAGCACCCAGACGCCTCGTCCGACGG
AGCGGCAGGGCCCCAACGATCCTCAACCAGCGCCCCACAGTGTGTGGTCGCGCCTCGGGGCCCGGCGA
CCGTCTTGCTCCCCCGAGCGGCACGGGGGCAAGGTGGCCCGCCTCCAACCCCCACCGACCAAAGCCCA
GCCTGCCCGCGGCGGACGCCGTGGGCGTCGCAGGGGTCGGGGTCGCGGTGGTCCCGGGGCCGCCGATG
GTTTGTCGGACCCCCGCCGGCGTGCCCCCAGAACCAATCGCAACCCGGGGGGACCCCGCCCCGGGGCG
GGGTGGACGGACGGCCCCGGCGCCCCCCATGGCGAGGCGTGGCGCGGAAGTGAGCAGCCCGACCCACC
CGGAGGCCCGCGGACACGGAGCGTGCGCCAAGCACCCCCCCCGCTAATGACGCTGGCGATTGCCCCCC
CGCCCGCGGACCCCCGCGCCCCGGCCCCGGAGCGAAAGGCGCCCGCCGCCGACACCATCGACGCCACC
ACGCGGTTGGTCCTGCGCTCCATCTCCGAGCGCGCGGCGGTCGACCGCATCAGCGAGAGCTTCGGCCG
CAGCGCACAGGTCATGCACGACCCCTTTGGGGGGCAGCCGTTTCCCGCCGCGAATAGCCCCTGGGCCC
CGGTGCTGGCGGGCCAAGGAGGGCCCTTTGACGCCGAGACCAGACGGGTCTCCTGGGAAACCTTGGTC
GCCCACGGCCCGAGCCTCTATCGCACTTTTGCCGGCAATCCTCGGGCCGCATCGACCGCCAAGGCCAT
GCGCGACTGCGTGCTGCGCCAAGAAAATTTCATCGAGGCGCTGGCCTCCGCCGACGAGACGCTGGCGT
GGTGCAAGATGTGCATCCACCACAACCTGCCGCTGCGCCCCCAGGACCCCATTATCGGGACGGCCGCG
GCGGTGCTGGATAACCTCGCCACGCGCCTGCGGCCCTTTCTCCAGTGCTACCTGAAGGCGCGAGGACT
CTGTGGACTCGATGAGCTCTGCTCTCGCCGACGGCTCGCCGATATCAAAGATATCGCCTCTTTTGTCT
TCGTGATCCTCGCTCGCTTGGCTAATAGAGTGGAAAGAGGAGTGGCCGAAATTGATTATGCCACATTG
GGCGTGGGCGTGGGCGAAAAAATGCACTTTTATTTACCTGGCGCTTGTATGGCTGGATTGATTGAGAT
TCTGGATACCCATAGACAAGAATGCTCCTCTAGAGTGTGTGAACTGACCGCTTCCCATATTGTGGCTC
CTCCCTATGTCCATGGAAAGTACTTCTACTGTAATAGTCTCTTCTAGGCTCTCGACAATCAACCTCTG
GATTACAAAATTTGTGAAAGATTGACTGGTATTCTTAACTATGTTGCTCCTTTTACGCTATGTGGATA
CGCTGCTTTAATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCGTGGTGTGCACT
GTGTTTGCTGACGCAACCCCCACTGGTTGGGGCATTGCCACCACCTGTCAGCTCCTTTCCGGGACTTT
CGCTTTCCCCCTCCCTATTGCCACGGCGGAACTCATCGCCGCCTGCCTTGCCCGCTGCTGGACAGGGG
CTCGGCTGTTGGGCACTGACAATTCCGTGGTGTTGTCGGGGAAATCATCGTCCTTTCCTTGGCTGCTC
GCCTGTGTTGCCACCTGGATTCTGCGCGGGACGTCCTTCTGCTACGTCCCTTCGGCCCTCAATCCAGC
GGACCTTCCTTCCCGCGGCCTGCTGCCGGCTCTGCGGCCTCTTCCGCGTCTCCGCCTTCGCCCTCAGA
CGAGTCGGATCTCTCTTTGGGCCGCCTCCCCGCCTGGTACCTTTAPGACCAPTGACTTACAPGGCAGC
TGTAGATCTTAGCCACTTTTTAAAAGAAAAGGGGGGACCTGGTACCTTTAAGACCAATGACTTACAAG
GCAGCTGTAGATCTTAGCCACTTTTTAAAAGAAAAGGGGGGACTGGAAGGGCTAATTCACTCCCAACG
AAGATAAGATCTGCTTTTTGCTTGTACTGGGTCTCTCTGGTTAGACCAGATCTGAGCCTGGGAGCTCT
CTGGCTAACTAGGGAACCCACTGCTTAAGCCTCAATAAAGCTTGCCTTGAGTGCTTCAAGTAGTGTGT
GCCCGTCTGTTGTGTGACTCTGGTAACTAGAGATCCCTCAGACCCTTTTAGTCAGTGTGGAAAATCTC
TAGCA

1. A recombinant replication-defective virus derived from the Herpesvirales order, wherein said virus is characterized by a deletion in a gene encoding ICP27, or a functional equivalent gene thereof, wherein said deletion is at least 1,200 bps in length and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp of the most 3′-end of said gene encoding ICP27 (SEQ ID NO: 11), or the functional equivalent gene thereof.

2. The recombinant replication-defective virus of claim 1, which is or is derived from a non-clinical (or laboratory) strain virus.

3. The recombinant replication-defective virus of claim 1 or 2, wherein said deletion comprises the entire coding sequence (or ORF) of said gene encoding ICP27 or said functional equivalent gene thereof.

4. The recombinant replication-defective virus of any one of claims 1-3, wherein said deletion further comprises the entire promoter region of said gene encoding ICP27 or said functional equivalent gene thereof, or a portion (e.g., the most 3′ about 400 nucleotides) of said promoter region.

5. The recombinant replication-defective virus of any one of claims 1-4, wherein said gene encoding ICP27 has the polynucleotide sequence of SEQ ID NO: 11.

6. The recombinant replication-defective virus of any one of claims 1-5, wherein said virus is derived from the Alloherpesviridae family or the Malacoherpesviridae family.

7. The recombinant replication-defective virus of any one of claims 1-5, wherein said virus is derived from the Herpesviridae family.

8. The recombinant replication-defective virus of claim 7, wherein said virus is derived from the Alphaherpesvirinae subfamily, the Betaherpesvirinae subfamily, or the Gammaherpesvirinae subfamily.

9. The recombinant replication-defective virus of claim 8, wherein said virus is derived from HHV-1 (Herpes Simplex Virus-1 or HSV-1), HHV-2 (Herpes Simplex Virus-2 or HSV-2), HHV-3 (Varicella Zoster Virus or VZV), HHV-4 (Epstein-Barr Virus or EBV), HHV-5 (Cytomegalovirus or CMV), HHV-6A/HHV-6B (Roseolovirus, Herpes Lymphotropic Virus), HHV-7, or HHV-8 (Kaposi's Sarcoma-Associated Herpesvirus or KSHV).

10. The recombinant replication-defective virus of claim 8, wherein said virus is derived from Cercopithecine herpesvirus-1 (CeHV-1) or Murid Herpesvirus 68 (MHV-68 or MuHV-4).

11. The recombinant replication-defective virus of claim 8, wherein said virus is derived from the Simplexvirus genus, such as Ateline herpesvirus 1, spider monkey herpesvirus, Porcine herpesviruses, Bovine herpesvirus 2, Cercopithecine herpesvirus 1 (Herpes B virus), Fruit bat alphaherpesvirus 1, Leporid herpesvirus 4, Macacine herpesvirus 1, Macropodid herpesvirus 2, and Papiine herpesvirus 2.

12. The recombinant replication-defective virus of claim 8, wherein said virus is derived from the Varicellovirus genus, such as Bovine herpesvirus 1, Bovine herpesvirus 5, Bubaline herpesvirus 1, Caprine herpesvirus 1, Canine herpesvirus 1, Cercopithecine herpesvirus 9, Cervid herpesvirus 1, Cervid herpesvirus 2, Elk herpesvirus 1, Equine herpesvirus 1, Equine herpesvirus 3, Equine herpesvirus 4, Equine herpesvirus 8, Equine herpesvirus 9, Feline herpesvirus 1, and Suid herpesvirus 1.

13. The recombinant replication-defective virus of claim 8, wherein said virus is derived from the Mardivirus genus, such as Anatid herpesvirus 1, Columbiform herpesvirus 1, Gallid herpesvirus 2, Gallid herpesvirus 3 (GaHV-3 or MDV-2), Meleagrid herpesvirus 1 (HVT), and Peacock herpesvirus 1.

14. The recombinant replication-defective virus of claim 8, wherein said virus is derived from the Litovirus genus, such as Gallid herpesvirus 1, and Psittacid herpesvirus 1.

15. The recombinant replication-defective virus of claim 8, wherein said virus is derived from a reptilian Alphaherpesvirus, such as Caretta caretta herpesvirus, Chelonid herpesvirus 1, Chelonid herpesvirus 2, Chelonid herpesvirus 3, Chelonid herpesvirus 4, Chelonia mydas herpesvirus, Coober herpesvirus, Emydid herpesvirus 1, Emydid herpesvirus 2, Fibropapilloma associated herpes virus, Gerrhosaurid herpesvirus 1, Gerrhosaurid herpesvirus 2, Gerrhosaurid herpesvirus 3, Glyptemis herpesvirus 1, Glyptemys herpesvirus 2, Iguanid herpesvirus 1, Iguanid herpesvirus 2, Loggerhead orocutaneous herpesvirus, Lung-eye-trachea associated herpesvirus, Pelomedusid herpesvirus 1, Red eared slider herpes virus, Terrapene herpesvirus 1, Terrapene herpesvirus 2, Testudinid herpesvirus 1, Testudinid herpesvirus 2, Testudinid herpesvirus 3, Testudinid herpesvirus 4, and Varanid herpesvirus 1.

16. The recombinant replication-defective virus of claim 8, wherein said virus is derived from the Rhadinovirus genus, such as Alcelaphine herpesvirus 1, Alcelaphine herpesvirus 2, Ateline herpesvirus 2, Bovine herpesvirus 4, Cercopithecine herpesvirus 17, Equine herpesvirus 2, Equine herpesvirus 5, Equine herpesvirus 7, Japanese macaque rhadinovirus, Leporid herpesvirus 1, and Murid herpesvirus 4 (Murine gammaherpesvirus-68 or MHV-68).

17. The recombinant replication-defective virus of claim 9, wherein said virus is a strain of HSV-1, such as KOS, KOS 1.1, KOS 1.1A, KOS63, KOS79, McKrae, Stain 17, F17, or McIntyre.

18. The recombinant replication-defective virus of any one of claims 1-17, wherein said functional equivalent gene thereof is ORF57 of KSHV, Mta/SM/EB2 of EBV, or UL69 of human CMV.

19. The recombinant replication-defective virus of any one of claims 1-18, further comprising a coding sequence for AAV Rep and Cap proteins, and/or a gene of interest (GOI) flanked by AAV ITR sequences.

20. The recombinant replication-defective virus of claim 19, wherein said coding sequence for said AAV Rep and Cap proteins, and/or said gene of interest (GOI) flanked by AAV ITR sequences is integrated into or replaces a non-essential gene of the virus (e.g., not required for viral replication and not required for viral packaging).

21. A recombinant vector capable of expressing ICP27 or a functional equivalent thereof in a host cell, said vector comprising:
(1) a coding sequence for said ICP27 or said functional equivalent thereof, operatively linked to a promoter capable of directing the transcription of said coding sequence in the host cell;
(2) a polyadenylation site 3′ to the coding sequence; and,
(3) optionally, one or more multi-cloning site(s);
wherein said vector contains no more than 300 bp, 250 bp, 200 bp, 150 bp, 100 bp, 50 bp, 30 bp, 20 bp, 10 bp, 9 bp, 8 bp, 7 bp, or 6 bp consecutive nucleotides of the virus of any one of claims 1-20.

22. The recombinant vector of claim 21, wherein said ICP27 has the amino acid sequence of SEQ ID NO: 10, or is at least 60%, 70%, 75%, 80%, 85%, 90%, 95%, 97%, 98%, 99%, 99.2%, 99.4%, 99.6%, or 99.8% identical to SEQ ID NO: 10.

23. The recombinant vector of claim 21 or 22, wherein the promoter comprises at least 400 polynucleotides.

24. The recombinant vector of claim 23, wherein the promoter comprises nucleotides 1-538 of SEQ ID NO: 11, nucleotides 127-538 of SEQ ID NO: 11, nucleotides 113, 139-113,550 of GenBank Accession No. KT887224, or nucleotides 113,013-113,550 of GenBank Accession No. KT887224.

25. The recombinant vector of any one of claims 21-24, wherein said coding sequence is partially or fully codon-optimized for expression in a mammalian host cell.

26. The recombinant vector of claim 25, wherein the most 3′ 300-350 nucleotides of the coding sequence are codon-optimized for expression in the mammalian host cell.

27. The recombinant vector of any one of claims 21-26, wherein said polyadenylation site is a bovine growth hormone (bGH) polyadenylation site.

28. The recombinant vector of any one of claims 21-27, wherein the coding sequence for said ICP27 comprises a mutation that reduces inhibition of host cell pre-mRNA splicing, while permitting HSV late gene expression.

29. The recombinant vector of claim 28, wherein the mutation is vBS3.3 double mutation, vBS4.3 double mutation, or vBS5.3 double mutation.

30. A host cell comprising the recombinant vector of any one of claims 21-29, wherein the host cell is capable of expressing said ICP27 or said functional equivalent thereof.

31. The host cell of claim 30, wherein the recombinant vector is stably integrated into the host cell genome.

32. The host cell of claim 30 or 31, which is a BHK cell, a Vero cell, or a HEK293 cell.

33. A method of propagating/amplifying/producing the recombinant replication-defective virus of any one of claims 1-20, the method comprising infecting the host cell of any one of claims 30-32 with the recombinant replication-defective virus of any one of claims 1-20.

34. The method of claim 33, further comprising harvesting the recombinant replication-defective virus of any one of claims 1-20 from the infected host cell of any one of claims 30-32.

35. The method of claim 33 or 34, wherein there is no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, or 2 bp sequence overlap between the recombinant replication-defective virus of any one of claims 1-20, and the coding sequence for said ICP27 or said functional equivalent thereof.

36. A method of producing a recombinant Adeno-Associated Virus (rAAV) comprising a gene of interest (GOI) coding sequence flanked by AAV ITR sequences, said method comprising co-infecting a production host cell with a first recombinant replication-defective virus of claim 19 or 20 comprising a coding sequence for AAV Rep and Cap proteins, and a second recombinant replication-defective virus of claim 19 or 20 comprising a gene of interest (GOI) flanked by AAV ITR sequences.

37. A method of producing a recombinant Adeno-Associated Virus (rAAV) comprising a gene of interest (GOI) coding sequence flanked by AAV ITR sequences, said method comprising infecting a production host cell with a recombinant replication-defective virus of claim 19 or 20 comprising a coding sequence for AAV Rep and Cap proteins, wherein the production host cell (1) comprises an integrated AAV pro-virus having the GOI coding sequence flanked by AAV ITR sequences; (2) is transfected by a vector (e.g., plasmid) having the GOI coding sequence flanked by the AAV ITR sequences; or (3) is co-infected with a rAAV having the GOI coding sequence flanked by the AAV ITR sequences.

38. The method of claim 36 or 37, wherein the production cell line is BHK, Vero, or HEK293.

39. The method of any one of claims 36-38, wherein the GOI is a functional equivalent of dystrophin (e.g., a dystrophin minigene encoding a functional micro-dystrophin protein).

40. The method of any one of claims 36-39, wherein the tropism of the AAV include skeletal muscle (such as AAV1, AAV6, AAV7, AAV8, or AAV9, preferably AAV9).

41. The method of any one of claims 36-40, wherein the gene of interest (GOI) includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS TUBA sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

42. The method of claim 41, wherein the GOI is a microdystrophin gene.

43. The method of claim 42, wherein the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272.

44. The method of claim 43, wherein the microdystrophin gene comprises a coding sequence for R16 and R17 spectrin-like repeats for the full length dystrophin protein (such as one described in U.S. Pat. No. 7,892,824).

45. The method of claim 44, wherein the microdystrophin gene comprises a coding sequence for the R1, R16, R17, R23, and R24 spectrin-like repeats of the full-length dystrophin protein (such as the microdystrophin gene described in PCT/US2016/013733).

46. A method of treating muscular dystrophy in a subject in need thereof, said method comprising administering to the subject a therapeutically effective amount of a recombinant AAV (rAAV) vector encoding a microdystrophin gene (such as one in any one of claims 43-45), wherein said rAAV is produced by the method of any one of claims 36-40).

47. The method of claim 46, further comprising producing said rAAV by the method of any one of claims 36-40, prior to administering to the subject the rAAV.

48. A method of making a recombinant replication-defective virus derived from the Herpesvirales order, wherein said virus is characterized by a deletion in a gene encoding ICP27, or a functional equivalent gene thereof, wherein said deletion is at least 1,200 bps in length and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp of the most 3′-end of said gene encoding ICP27 (e.g., SEQ ID NO: 11), or the functional equivalent gene thereof, said method comprising creating said deletion of said gene encoding ICP27 or said functional equivalent gene thereof by homologous recombination in a host cell.

49. The method of claim 48, wherein the homologous recombination is carried out by using a bacterial artificial chromosome (BAC) comprising the genome of said virus derived from the Herpesvirales order (e.g., HSV genome) having said gene encoding ICP27 or said functional equivalent gene thereof.

50. The method of claim 48 or 49, wherein the host cell is an E. coli, or a eukaryotic cell such as a yeast, an insect cell (e.g., SF9), or a mammalian cell (e.g., a Vero cell, a baby hamster kidney (BHK) cell, a HeLa cell, a human lung fibroblast MRC-5, a human foreskin fibroblast (HFF), a human embryonic lung fibroblast (HELF), a Madin-Darby canine Kidney cell (MDCK), a Madin-Darby bovine kidney cell (MDBK), or others).


SEQUENCE LISTING (13 sequences)

SEQ ID NO: 1 - DNA, 2188 bp, Artificial Sequence
  source: /note=“Description of Artificial Sequence Synthetic polynucleotide”
ggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttaggctagcgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 2 - DNA, 2188 bp, Artificial Sequence
  source: /note=“Description of Artificial Sequence Synthetic polynucleotide”
ggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggactctgtggactcgatgagctctgctctcgccgacggctcgccgatatcaaagatatcgcctcttttgtcttcgtgatcctcgctcgcttggctaatagagtggaaagaggagtggccgaaattgattatgccacattgggcgtgggcgtgggcgaaaaaatgcacttttatttacctggcgcttgtatggctggattgattgagattctggatacccatagacaagaatgctcctctagagtgtgtgaactgaccgcttcccatattgtggctcctccctatgtccatggaaagtacttctactgtaatagtctcttctaggctagcgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 3 - DNA, 2188 bp, Artificial Sequence
  source: /note=“Description of Artificial Sequence Synthetic polynucleotide”
ggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggccactgatattgacatgcttatcgaccttggactcgatctctccgactcggacttggatgaggacccacctgagccggccgaaagccgccgggacgatctggagagcgactccaacggcgaatgctcgtcctctgacgaagatatggaagatccgcacggcgaagatggcccggagccgattctcgacgccgcccggcccgccgtgcgcccatcacggcctgaagatcccggtgtcccatccacccaaactccgcggcccaccgagcgccagggcccgaatgacccccagccggctccgcattccgtgtggagccgcctgggagccagacgcccttcatgctcccctgagcggcacgggggaaaggtcgcgcggctgcaacctcccccgaccaaggcccagcctgcccgcggtggacgccgggggcgccggagaggtcgcggcaggggtggcccgggggccgcagacggactgtccgatccgcggcggagggcgcccagaacgaaccggaaccccgggggccctagacctggagccggatggacagacggacccggagccccacatggcgaagcgtggagaggctcagagcagcctgaccctccgggtggcccgaggacccgcagcgtgcggcaggcaccaccaccccttatgaccctcgccattgcgccacccccggccgatccgcgcgctccggcccccgagagaaaggcccccgccgccgataccatcgatgctaccacccgcctggtcctgcggtccatcagcgagagagccgcagtggaccgcatctccgaatccttcggccggtcggcacaggtcatgcacgacccgtttggtggacagccctttcctgccgctaactccccgtgggcacccgtgctcgcgggacagggcggccctttcgacgcggaaaccagaagagtcagctgggagactctggtggcccacggaccgtccctgtaccggaccttcgccggaaacccgagggccgccagcactgccaaggccatgcgggactgtgtgctgcgccaggaaaacttcatcgaagcactcgcctccgccgacgaaaccctggcctggtgcaagatgtgtattcaccataatcttcctctgcggcctcaagaccccattatcgggactgcggcggccgtgttggacaacctggcgacccgcctgcggccgttcctgcaatgctacctgaaagccaggggactgtgcggactggacgaattgtgctcgcggcgccgcctcgcggacatcaaggacatcgcttccttcgtgttcgtgatcctggccagactcgccaaccgagtggagaggggagtggcagaaattgactacgcgactctgggggtcggagtgggagagaagatgcacttctacctccctggcgcctgcatggctggactgatcgagatcctggacacccatagacaggaatgttcatcccgcgtgtgcgagctgactgcgtcgcacatcgtggctcccccctatgtgcacgggaagtacttctactgcaacagcctgttctaagctagcgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 4 - DNA, 2444 bp, Artificial Sequence
  source: /note=“Description of Artificial Sequence Synthetic polynucleotide”
ggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttaggtacaataaaaacaaaacatttcaaacaaatcgccccacgtgttgtccttctttgctcatggccggcggggcgtgggtcacggcagatggcgggggtgggcccggcgtacggcctgggtgggcggagggaactaacccaacgtataaatccgtccccgctccaaggccggtgtcatagtgcccttaggagcttcccgcccgggcgcatccccccttttgcactatgacagcgacccccctcaccaacctgttcttacgggccccggacataacccacgtggcccccccttactgcctcaacgccacctggcaggccgaaacggccatgcacaccagcaaaacggactccgcttgcgtggccgtgcggagttacctggtccgcgcctcctgtgagaccagcggcacaatccactgctttttctttgcggtatacaaggacacccaccatacccctccgctgattaccgagctccgcaactttgcggacctggtt

SEQ ID NO: 5 - DNA, 2314 bp, Artificial Sequence
  source: /note=“Description of Artificial Sequence Synthetic polynucleotide”
cgtcacatccaggccggcggaaaccggaacggcatatgcaaactggaaactgtcctgtcttggggcccacccacccgacgcgtcatatgtaaatgaaaatcgttcccccgaggccatgtgtagcctggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggactctgtggactcgatgagctctgctctcgccgacggctcgccgatatcaaagatatcgcctcttttgtcttcgtgatcctcgctcgcttggctaatagagtggaaagaggagtggccgaaattgattatgccacattgggcgtgggcgtgggcgaaaaaatgcacttttatttacctggcgcttgtatggctggattgattgagattctggatacccatagacaagaatgctcctctagagtgtgtgaactgaccgcttcccatattgtggctcctccctatgtccatggaaagtacttctactgtaatagtctcttctaggctagcgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 6 - DNA, 2188 bp, Artificial Sequence
  source: /note=“Description of Artificial Sequence Synthetic polynucleotide”
ggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggccaccgacatcgacatgctgatcgatttaggtttagatttaagcgacagcgatctggacgaggacccccccgaacccgctgaatctcgtcgggacgatttagagtccgactccaatggagagtgtagcagcagcgacgaggacatggaagaccctcacggcgaggatggccccgaacctattctcgatgctgctcggcccgctgtgaggccttcccggcccgaagatcccggtgtgcctagcacccaaacccctcgtcctaccgagcggcaaggtcccaatgatcctcagcccgcccctcatagcgtctggtctcgtctgggagctaggaggcctagctgttcccccgaacggcacggaggcaaagtggctaggctgcagcctccccccaccaaagctcaacccgctcggggaggccggaggggtcgtcggcggggtcgtggaaggggcggccccggtgctgccgacggactgagcgatcctaggaggagggcccctcggaccaatcgtaatcccggtggacctcgtcccggtgctggatggaccgatggacccggtgctcctcacggagaggcttggaggggaagcgagcagcccgatcctcccggtggccctaggacaaggagcgttcgtcaagctcctcctcctctcatgactttagccattgcccctcctcccgctgatcctagggctcccgctcccgaaaggaaagcccccgccgccgataccattgacgccacaactcgtctcgtgctgaggtccatttccgaacgggccgccgtcgatcgtatcagcgagagcttcggaaggtccgcccaagttatgcacgatcccttcggcggccaaccctttcccgctgctaatagcccttgggcccccgtgctggctggacaaggaggccctttcgacgccgagactcgtagggtgagctgggagacactggtggcccatggcccttctttataccggacattcgctggcaaccctcgtgctgccagcacagctaaggccatgcgggactgtgtgctgcggcaagaaaacttcattgaggctttagccagcgctgatgagactttagcttggtgcaagatgtgcatccaccacaatttacctctgaggccccaagatcccatcattggcacagccgccgccgtgctggataatttagccactcgtctccggccctttctgcagtgctacctcaaagcccggggtttatgcggactcgatgagctgtgttctcgtcggaggctggccgacatcaaggacatcgccagcttcgtgttcgtgatcctcgctcgtctggccaatcgtgtggagaggggagtggccgaaatcgattatgccaccttaggcgtgggcgttggcgagaagatgcacttttatttacccggtgcttgtatggccggactcattgagatcctcgatacccaccggcaagaatgctcctctcgtgtgtgcgagctgaccgcttcccacattgtggcccccccctacgtgcacggaaaatacttctactgtaactctttattctgagctagcgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 7 - DNA, 2188 bp, Artificial Sequence
  source: /note=“Description of Artificial Sequence Synthetic polynucleotide”
ggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggctacagacatcgacatgctgatcgacctgggcctcgacctgtctgactctgatctggacgaagatcctcctgagcctgccgagtccagaagggacgacctggaatctgactctaacggcgagtgctcctccagcgacgaggatatggaagatccacatggcgaggacggccctgagcctattttggatgctgccagacctgccgtgcggccttctagacctgaagatcctggcgtgccatctacacagacccctagacctacagagcggcagggccctaatgatcctcagcctgctcctcactccgtgtggtctagattgggagccagacggccttcctgctctcctgaaagacatggcggcaaggtggcaagactgcagcctcctccaacaaaggctcaacctgctagaggcggcagaaggggcagacgtagaggtagaggaagaggtggacctggcgctgctgatggcttgtctgatcctagaagaagggcccctcggaccaacagaaatcctggtggacctagaccaggcgccggatggacagatggtccaggtgctcctcatggcgaagcttggagaggatctgagcaacctgatcctccaggcggccctagaaccagatctgttagacaggctcctcctcctctgatgaccctggctattgctccacctcctgccgatcctagagcacccgctccagaaagaaaggcccctgctgctgacaccatcgacgctacaacaagactggtgctgcggtccatctctgagagggccgctgtggacagaatctccgagtctttcggccgctctgcccaagtgatgcacgatccttttggcggccagccttttcctgccgccaattctccttgggctcctgtgttggctggccaaggcggaccttttgacgccgagacaagaagagtgtcctgggagacactggtggctcacggacctagcctgtacagaaccttcgctggcaaccctagagccgcttctaccgccaaggccatgagagactgtgtgctgagacaagagaacttcatcgaggccctggcctctgccgatgagactctggcttggtgcaagatgtgtatccaccacaacctgcctctgcggcctcaggaccctatcattggaacagctgccgccgtgctggataacctggctaccagactcagacccttcctgcagtgctacctgaaggctagaggcctgtgtggcctggacgagctgtgctccagaagaaggctggctgatatcaaggatatcgcctccttcgtgttcgtgatcctggccagactggctaacagagtggaaagaggcgtggccgagatcgattatgctaccctcggagtcggcgtgggcgagaagatgcatttttacctgcctggcgcctgcatggccggactgatcgagattctggatacccaccggcaagagtgctccagcagagtgtgtgaactgaccgcctctcacatcgtggctcctccatacgtgcacggcaagtacttctactgcaactccctgttctgagctagcgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 8 - DNA, 2314 bp, Artificial Sequence
  source: /note=“Description of Artificial Sequence Synthetic polynucleotide”
cgtcacatccaggccggcggaaaccggaacggcatatgcaaactggaaactgtcctgtcttggggcccacccacccgacgcgtcatatgtaaatgaaaatcgttcccccgaggccatgtgtagcctggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttaggctagcgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 9 - DNA, 2188 bp, Artificial Sequence
  source: /note=“Description of Artificial Sequence Synthetic polynucleotide”
ggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggatgaactgtgctctagaagaaggctggccgatatcaaggatatcgcctccttcgtgttcgtgatcctggctcggctggccaacagagtggaaagaggcgtggccgagatcgactatgctaccctcggagttggcgtgggcgagaagatgcacttttacctgcctggcgcctgtatggccggcctgatcgagatcctggacacccacagacaagagtgctcctccagagtgtgcgagctgaccgcttctcacatcgtggctcctccatacgtgcacggcaagtacttctactgcaactccctgttctgagctagcgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 10 - protein, 512 aa, Herpes simplex virus 1
MATDIDMLIDLGLDLSDSDLDEDPPEPAESRRDDLESDSNGECSSSDEDMEDPHGEDGPEPILDAARPAVRPSRPEDPGVPSTQTPRPTERQGPNDPQPAPHSVWSRLGARRPSCSPERHGGKVARLQPPPTKAQPARGGRRGRRRGRGRGGPGAADGLSDPRRRAPRTNRNPGGPRPGAGWTDGPGAPHGEAWRGSEQPDPPGGPRTRSVRQAPPPLMTLAIAPPPADPRAPAPERKAPAADTIDATTRLVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTAAAVLDNLATRLRPFLQCYLKARGLCGLDELCSRRRLADIKDIASFVFVILARLANRVERGVAEIDYATLGVGVGEKMHFYLPGACMAGLIEILDTHRQECSSRVCELTASHIVAPPYVHGKYFYCNSLF

SEQ ID NO: 11 - DNA, 2077 bp, Herpes simplex virus 1
cgtcacatccaggccggcggaaaccggaacggcatatgcaaactggaaactgtcctgtcttggggcccacccacccgacgcgtcatatgtaaatgaaaatcgttcccccgaggccatgtgtagcctggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 12 - DNA, 1881 bp, Artificial Sequence
  source: /note=“Description of Artificial Sequence Synthetic polynucleotide”
ccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggactctgtggactcgatgagctctgctctcgccgacggctcgccgatatcaaagatatcgcctcttttgtcttcgtgatcctcgctcgcttggctaatagagtggaaagaggagtggccgaaattgattatgccacattgggcgtgggcgtgggcgaaaaaatgcacttttatttacctggcgcttgtatggctggattgattgagattctggatacccatagacaagaatgctcctctagagtgtgtgaactgaccgcttcccatattgtggctcctccctatgtccatggaaagtacttctactgtaatagtctcttctag

SEQ ID NO: 13 - DNA, 2929 bp, Artificial Sequence
  source: /note=“Description of Artificial Sequence Synthetic polynucleotide”
ggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggactctgtggactcgatgagctctgctctcgccgacggctcgccgatatcaaagatatcgcctcttttgtcttcgtgatcctcgctcgcttggctaatagagtggaaagaggagtggccgaaattgattatgccacattgggcgtgggcgtgggcgaaaaaatgcacttttatttacctggcgcttgtatggctggattgattgagattctggatacccatagacaagaatgctcctctagagtgtgtgaactgaccgcttcccatattgtggctcctccctatgtccatggaaagtacttctactgtaatagtctcttctaggctctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctccgccttcgccctcagacgagtcggatctctctttgggccgcctccccgcctggtacctttaagaccaatgacttacaaggcagctgtagatcttagccactttttaaaagaaaaggggggacctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca